(19) United States
(12) Patent Application Publication
(10) Pub. No.: US 2022/0228145 A1
(43) Pub. Date: Jul. 21, 2022

(54) A Ribozyme Comprising a Target-Binding Domain

(71) Applicant: AGENCY FOR SCIENCE, TECHNOLOGY AND RESEARCH, Singapore (SG)

(72) Inventors: Sherry Aw, Singapore (SG); Chong Hui Koh, Singapore (SG); Yu Theng Mandy Lim, Singapore (SG)

(21) Appl. No.: 17/602,411

(22) PCT Filed: Apr. 13, 2020
(86) PCT No.: PCT/SG2020/050226

§ 371 Date: Oct. 8, 2021

(30) Foreign Application Priority Data
Apr. 12, 2019 (SG) 10201903323U

(51) Int. Cl. C12N 15/113; C12Q 1/6888; C12Q 1/6823; C12Q 1/70

(52) U.S. Cl. CPC C12N 15/113; C12Q 1/6888; C12Q 1/6823; C12N 2310/3517; C12Q 2600/178; C12Q 2600/112; C12Q 1/70

(57) ABSTRACT

A ribozyme comprising a target-binding domain, a polynucleotide encoding the same, a kit comprising the same, and uses thereof.

### CROSS-REFERENCE TO RELATED APPLICATIONS

This application claims the benefit of priority of Singapore provisional application No. 10201903323U, filed on 12 Apr. 2019, the contents of it being hereby incorporated by reference in its entirety for all purposes.

### FIELD OF THE INVENTION

The present invention relates to the field of biochemistry, in particular molecular biology. In particular, the present invention relates to a ribozyme engineered to comprise one or more target-binding domains.

### BACKGROUND OF THE INVENTION

While RNA used to be thought of as merely an intermediary messenger between the inherited genetic code (DNA) and its functional output (protein), it is now abundantly clear that the levels and profiles of both coding and non-coding RNA in cells and in individuals present substantial information beyond its messenger function. Although doctors have traditionally relied on patient history, symptoms, biopsies, and other procedures to form a diagnosis, with the advent of advanced sequencing and personalized medicine, the detection and measurement of various types of RNA molecules are now increasingly important to guide clinical decision-making.

One class of RNA molecules that is of particular interest is microRNA (miRNA). miRNAs are endogenous short, non-coding RNAs that bind to and down-regulate target mRNAs, which are dysregulated in many human diseases. miRNA expression signatures are correlated with patient diagnosis, staging, prognosis and response. Importantly, miRNAs are found circulating in the blood, allowing for their detection with minimally invasive procedures. Data related to miRNA signatures of disease and treatment response continue to grow, and there have been more than 100 clinical trials to date that incorporate miRNAs as biomarkers. While there are clinical methods for measuring miRNAs (mostly based on RT-qPCR), due to the often extremely low concentrations of miRNAs in biological samples (such as the serum), the samples often first require RNA purification and concentration prior to detection. These require reverse transcription via a DNA intermediate. Therefore, tools and methods which could directly amplify target miRNAs or their signals in the biological sample, without using protein enzymes or employing DNA intermediates, could advance our capabilities for cheap and efficient RNA detection.

The ability to detect RNA molecules is also critical in diagnosing viral infections. There are at least 47 families of RNA viruses. Several cause illnesses with substantial disease burden: Severe acute respiratory syndrome coronavirus 2 (SARS-CoV-2)(which causes COVID-19), Ebola, SARS, West Nile fever, hepatitis C, influenza and measles, while others are linked to plant viruses that cause severe crop losses, e.g. potato virus Y (PVY). During viral disease outbreaks, sensitive, convenient, reliable and fast methods for diagnosis are paramount for rapid containment. Current routine methods for diagnosis of infections by RNA viruses are mainly PCR-based (especially for novel viruses; serological tests can be developed but are not typically of widespread use), and require RNA in samples to first be isolated and reverse transcribed into cDNA, and then amplified with specific primers, before the amplified product is detected using fluorescent dyes using RT-qPCR. This requires expensive reagents, specialized equipment and advanced skill sets for analysis. This slows down diagnosis during outbreaks, as samples must be sent to specialized laboratories. CRISPR-based technologies like DETECTR (Chen et. al., CRISPR-Cas12a target-binding unleashes indiscriminate single-stranded DNase activity Science 2018) and SHERLOCK (Gootenberg, J. S. et al. Multiplexed and portable nucleic acid detection platform with Cas13, Cas12a, and Csm6. Science 360, 439-444, doi:10.1126/science.aaq0179 (2018)) show promise for point-of-care (POC) detection, but require amplification using expensive isothermal amplification enzymes and CRISPR reagents. A major roadblock in the process of developing a low-cost diagnostic for viral RNA is the lack of a method to directly amplify and detect RNA without enzymes and going through a DNA intermediate.

In sum, there is a need to provide simple and efficient tools for the amplification and detection of target RNA molecules or their signals which will enable fast, efficient detection of RNA markers and viral RNA for different diagnostic applications.

### SUMMARY OF THE INVENTION

In one aspect, the present disclosure refers to a ribozyme comprising: a) one or more catalytic domains capable of switching between an active state and an inactive state; b) one or more releasable RNA segments, wherein each of said releasable RNA segment is flanked by two ribozyme cleavage sites, wherein each cleavage site is cleaved by at least one of the one or more catalytic domains in an active state; c) one or more target-binding domains, each for the binding of a target RNA molecule; wherein each of the one or more catalytic domains is linked to one of the one or more target-binding domains, wherein the catalytic domain is in an inactive state when the target-binding domain linked to said catalytic domain is not bound by the target RNA molecule, and wherein the catalytic domain is in an active state when the target-binding domain linked to said catalytic domain is bound by the target RNA molecule; and wherein when both cleavage sites flanking a releasable RNA segment are cleaved by the one or more catalytic domains, the one or more releasable RNA segment is released from the ribozyme.

In another aspect, the present disclosure refers to a method of detecting presence of a target RNA molecule in a sample, wherein the method comprises: a) incubating the sample with an ribozyme of the present disclosure at temperature T1 which allows the binding of the target RNA molecule for binding with one or more target-binding domains comprised in the ribozyme; b) incubating the sample at temperature T2 which allows the target RNA molecule and the releasable RNA segment to be released from the ribozyme; c) detecting the release of the releasable RNA segment from the ribozyme.

In yet another aspect, there is also provided a method of amplifying a target RNA molecule, wherein the method comprises: a) incubating the target RNA molecule with an ribozyme of the present disclosure at temperature T1 which allows the binding of the target RNA molecule with one or more target-binding domains comprised in the ribozyme, and wherein the releasable RNA segment comprises a sequence identical to the target RNA molecule; b) incubating the ribozyme bound to the target RNA molecule at temperature T2 which allows the target RNA molecule and the RNA segment to be released from the ribozyme. In a further example, steps a) to b) are repeated for one or more times, wherein the target RNA molecules and the releasable RNA segment released from step b) are for binding to another copy of the ribozyme.

In still another aspect, there is provided a method of diagnosing a disease in a subject, the method comprising: a) obtaining a sample from the subject; b) incubating the sample with the ribozyme of the present disclosure at temperature T1 which allows the binding of a disease associated target RNA molecule with one or more target-binding domains comprised in the ribozyme; c) incubating the sample at temperature T2 which allows the target RNA molecule and the releasable RNA segment to be released from the ribozyme; d) detecting the levels of the releasable RNA segment from the ribozyme.

In yet another aspect, there is provided a polynucleotide encoding the ribozyme of the present disclosure. In some examples where the ribozyme is comprised of more than one RNA strands, the RNA strands can be encoded together on one polynucleotide or separately on several polynucleotides.

In a further aspect, there is further provided a kit comprising the ribozyme of the present disclosure.

### BRIEF DESCRIPTION OF THE DRAWINGS

The invention will be better understood with reference to the detailed description when considered in conjunction with the non-limiting examples and the accompanying drawings, in which:

FIGS. 1A-1I. Exemplary and non-limiting structures formed by the ribozyme of the present disclosure. The labeling of motifs is consistent with that of structures I-V in the detailed description. (FIGS. 1A-1C) Examples of the ribozyme formed by one RNA strand and having one target-binding domain. The ribozyme of panel A is characterized by two 4-way junctions formed on either side of the releasable RNA segment (motif [E]), with the catalytic domain (motifs [C] and [c]), optional inhibitory domain (motifs [B] and [b]), and target binding domain (motifs [A] and [a]) based on the extension of one of the helices of one of the 4-way junction structures. The ribozymes of FIGS. 1B and 1C adopt a similar structure to FIG. 1A, except that the 4-way junction formed by the region between motif [D] and motif [e] is replaced with a 3-way junction (FIG. 1B) or a 2-way junction (FIG. 1C). (FIGS. 1D-1I) Examples of the ribozyme formed by two RNA strands and having two target-binding domains. The ribozyme of panel D FIG. 1D is characterized by two 4-way junctions formed on either side of the releasable RNA segment, with each side comprising a catalytic domain, an optional inhibitory domain, and a target binding domain. The ribozyme of FIGS. 1E and 1F adopt a similar structure to panel D FIG. 1D, except that the 4-way junctions are replaced by 3-way junctions and 2-way junctions. The ribozyme of each of FIGS. 1D-1F can be considered a tandem ribozyme reverse-joined by two “single ribozymes” (hence “reverse-joined tandem ribozyme”), with the two “single ribozymes” displaying approximately “mirror-image” orientations. While the ribozyme of FIGS. 1G-1I can also be considered a tandem ribozyme joined by two “single ribozymes”, the two “single ribozymes” adopt the same orientation (hence named “duplicated tandem ribozyme”). The ribozyme of FIG. 1G is characterized by two 4-way junctions formed on either side of the releasable RNA segment. The ribozyme of FIGS. 1H and 1I adopt a similar structure to the ribozyme of FIG. 1G, except that one of the 4-way junctions is replaced by a 3-way junction (FIG. 1H) or by a 2-way junction (FIG. 1I). The arrows indicate the cleavage sites on the ribozyme.

FIG. 2. Working principle of the ribozyme as an RNA sensor and amplifier. The variable motif [b] connecting motif [c] (of the catalytic domain) to the motif [a] (of the target-binding domain) can be designed to have complementarity to motif [C] of the catalytic domain. Top: Without the target RNA molecule, the binding between [b] and [C] acts as a “toehold” to minimize self-cleavage, preventing cleavage of the cleavage site [D] and the release of the releasable RNA segment. Bottom: With the binding of target RNA molecules to the target-binding domains, the catalytic domains are activated, resulting in cleavage at the cleavage site and the releasable RNA segment (the sequence of this product can be varied, which in some examples exhibit loose complementarity to the ribozyme so that cleavage is favoured over re-ligation). The final cleavage product comprises the releasable RNA segment flanked by 2-4 nucleotides from the cleavage sites. The arrowheads indicate the cleavage sites.

FIG. 3. Working principle of the ribozyme as an RNA sensor and amplifier as illustrated using a specific example with nucleotides labelled. In this example, the release of both the RNA cleavage product and target RNA molecules can be facilitated at a melting temperature that can be determined based on the length and degree of the complementary regions. The released target RNA molecules can bind anew to uncleaved ribozymes (present in excess) in a new cycle. As the ribozyme is in excess, a given copy number of target RNA molecules can activate self-cleavage of ribozymes, leading to the release of multiple cleavage products, hence amplifying the target RNA signal.

FIG. 4. Self-cleavage by an exemplary ribozyme (with microRNA dme-ban-5p as its target RNA molecule) directly amplifies RNA signal by cleaving out increasing levels of of the releasable RNA fragment (in this example ban-3pR RNA). Increasing number of cycles (from 3 to 5 to 10 cycles), or increasing levels of target RNA (from 0 (Water only) to 20 nM to 50 nM), leads to increase in amount of cleavage product, hence amplifying target RNA. 200 nM of ribozyme is provided in each reaction.

FIG. 5. Further increase in cycle number (from 10 to 20 to 30 cycles) or increase in length of cycle time (from 15 to 30 min) leads to greater fold-increase in amount of cleavage product produced by an exemplary ribozyme (with microRNA dme-ban-5p as its target RNA molecule), and further amplification of target RNA. 200 nM of the ribozyme and 50 nM of target RNA molecules are provided in each reaction, i.e. ratio of ribozyme:Target is 4:1.

FIG. 6. The ribozyme can amplify human microRNAs, demonstrated here with for an exemplary ribozyme with hsa-let-7f as its target RNA molecule. The ribozyme is able to amplify the target RNA molecule by ˜200-fold, calculated by normalizing all cleavage product bands based on the S2 loading controls, and subtracting the background cleavage in the control (water) lane. 200 nM of ribozyme, and either 50 nM, 10 nM or 0.1 nM of target miRNA was added for each reaction.

FIG. 7. The ribozyme with human microRNA hsa-let-7f as its target is specific for its target, and is able to detect and amplify its target from within a complex mixture of RNAs. While addition of hsa-let-7f more strongly activates the ribozyme compared to no target (water control), addition of 50 nM of a dissimilar miRNA sequence (hsa-miR-122-5p) did not increase cleavage activity beyond background cleavage, indicating that the ribozyme was not activated. When equal concentrations of hsa-let-7f-5p and hsa-miR-122-5p were added (50 nM each), the ribozyme retained its specific cleavage towards hsa-let-7f-5p. When either 10-fold, 100-fold, or 1000-fold of total RNA from Drosophila melanogaster was added to the reaction, the ribozyme was still able to detect and amplify hsa-let-7f-5p. 200 nM of ribozyme was provided in each reaction.

FIG. 8. The ribozyme can detect and amplify target RNA molecules that are 40 nt, 60 nt and 80 nt in length, containing RNA sequence fragments of genes (Orf1ab and E gene) from the genome of coronavirus SARS-CoV-2. 200 nM of ribozyme and 50 nM of target RNA are provided in each reaction. See Methods for additional details.

FIG. 9. The cleavage product from the ribozyme can activate the Pandan fluorescent sensor. In this example, the cleavage product is dme-ban-3p, and the ribozyme's target RNA molecule is dme-ban-5p. The cleavage product can activate Pandan fluorescent sensor for dme-ban-3p, compared to the catalytic mutant that is unable to cleave even in presence of the target. Fluorescence experiments were carried out in triplicate.

FIGS. 10A and 10B. The single catalytic domain configuration is capable of cleaving two cleavage sites to cause release of the RNA product. (FIG. 10A) “Single catalytic domain” design for detection and amplification of microRNA dme-ban-5p, to cleave and release ban3p-reverse RNA. Two “single catalytic domain” configurations are shown (i and ii). (FIG. 10B) Both designs 1 and 2 are effective in releasing the cleavage products upon incubation with the target RNA molecules.

FIG. 11. Melting temperatures ranging from 50° C. to 90° C. were tested in the cleavage reactions for ribozymes targeting dme-ban-5p (the target RNA molecule). 200 nM of ribozyme and 50 nM of target RNA molecule were provided in each cleavage reaction. Ribozyme cleavage was carried out at 37° C.

FIG. 12. Construction of an exemplary ribozyme. The sequences and primers used in the construction of a specific ribozyme have been mapped onto the ribozyme structure, showing the design and construction method.

### DETAILED DESCRIPTION OF THE PRESENT INVENTION

The present invention provides a ribozyme useful for the sensing and amplification of RNAs.

In a first aspect, the present invention refers to a ribozyme comprising: a) one or more catalytic domains capable of switching between an active state and an inactive state; b) one or more releasable RNA segments, wherein each of said releasable RNA segment is flanked by two ribozyme cleavage sites, wherein each cleavage site is cleaved by at least one of the one or more catalytic domains in an active state; c) one or more target-binding domains, each for the binding of a target RNA molecule; wherein each of the one or more catalytic domains is linked to one of the one or more target-binding domains, wherein the catalytic domain is in an inactive state when the target-binding domain linked to said catalytic domain is not bound by the target RNA molecule, and wherein the catalytic domain is in an active state when the target-binding domain linked to said catalytic domain is bound by the target RNA molecule; and wherein when both cleavage sites flanking a releasable RNA segment are cleaved by the one or more catalytic domains, the one or more releasable RNA segment is released from the ribozyme.

As used herein, the term “ribozyme” refers to an RNA molecule that is capable of catalyzing specific biochemical reactions. Common examples of such reactions include the cleavage or ligation of RNA and DNA and peptide bond formation. The term “ribozyme” as used herein includes both natural and artificial ribozymes. Artificial ribozymes include synthetic ribozymes and ribozymes modified or engineered from natural ribozymes. The term “ribozyme” also encompass ribozyme fusions or ribozyme complexes derived from natural or artificial ribozymes.

As used herein, the term “catalytic domain” refers to the domain within a ribozyme that is responsible for catalyzing the biochemical reactions as mentioned above. In one example, in a ribozyme capable of cleaving RNA, the catalytic domain is the domain responsible for catalyzing the cleavage of the RNA backbone at a ribozyme cleavage site. The term “ribozyme cleavage site” refers to the sequences recognized and cleaved by a ribozyme catalytic domain. Unless specified otherwise, the term “cleavage site” as used herein refers a ribozyme cleavage site. A catalytic domain is in an “active state” when it is capable of catalyzing the biochemical reaction; whereas a catalytic domain is in an “inactive state” when it is incapable of catalyzing the biochemical reaction. In a further example, a catalytic domain is in an “active state” when it is capable of cleaving a ribozyme cleavage site; whereas a catalytic domain is in an “inactive state” when it is incapable of cleaving a ribozyme cleavage site.

The term “target-binding domain” refers to a domain which is capable of binding a target RNA molecule. In an example, the binding between the target RNA molecule and the target-binding domain occurs through the annealing of complementary sequences between the two. Thus, it is possible to design or modify the sequence of the one or more target-binding domains so that they can bind to different target RNA molecules with specific sequences.

The term “complementary” as used herein describes a relationship between two nucleotides or two polynucleotides. When referring to RNA complementarity, the nucleotide A is complementary to the nucleotide U, and vice versa, and the nucleotide C is complementary to the nucleotide G, and vice versa. Complementary nucleotides include those that undergo Watson and Crick base pairing and those that base pair in alternative modes. It should be understood that, unless explicitly specified (e,g. by assigning a percentage or the term “fully” or “partially), the term “complementary” when used in relation to a polynucleotide (more than 2 nucleotides in length), includes varying degrees of complementarity. As used herein, the term “complementarity” refers to the degree and pattern by which one RNA strand or segment is complementary to another RNA strand of segment. When a percentage is assigned to a “complementarity” or a “degree of complementarity” between two polynucleotides (or segments thereof), the percentage refers to the percentage of nucleotides in one polynucleotide (or a segment thereof) that are complementary to the other polynucleotide (or a segment thereof). Therefore, a reference to two polynucleotide strands being “complementary” should be understood to cover both full and partial complementarity.

The term “target RNA molecule” as used herein refers to RNA molecules of interest that are to be sensed and bound by the target-binding domain. Examples of target RNA molecules include but are not limited to, viral RNA, microRNA (miRNA), short interfering RNA (siRNA), small RNA (sRNA), messenger RNA (mRNA), non-coding RNA (ncRNA), short non-coding RNA, transfer RNA (tRNA), ribosomal RNA (rRNA), transfer-messenger RNA (tmRNA), clustered regularly interspaced short palindromic repeats RNA (CRISPR RNA), antisense RNA, pre-mRNA and pre-miRNA.

The term “linked” refers to the relationship between two domains, and can refer to physical linkage, functional linkage, or both. In one example of the present invention, a catalytic domain is linked to a target-binding domain when the active/inactive state of the catalytic domain is determined by the state of the target-binding domain, specifically whether the target-binding domain is bound to its corresponding target RNA molecule.

The term “flanked” refers to a polynucleotide sequence that is adjacent to another sequence or that is in between an upstream polynucleotide sequence and/or a downstream poylnucleotide sequence, i.e., 5′ and/or 3′, relative to the sequence. For example, “a releasable RNA segment that is “flanked” by two cleavage sites” indicates that one cleavage site is located 5′ to the releasable RNA segment and the other cleavage site is located 3′ to the releasable RNA segment; however, there may be intervening sequences therebetween.

As the cleavage sites are comprised on the ribozyme itself, the ribozyme of the present disclosure is considered a self-cleaving ribozyme, and the “releasable RNA segment” can be considered a cleavage product of the self-cleaving activity. As the release of the releasable RNA segment from the ribozyme is a result of the ribozyme binding with its one or more target RNA molecules, the ribozyme can be used to detect the presence of target RNA molecules. As the target RNA molecules can be released from the ribozyme to activate more ribozymes and trigger the release of more “releasable RNA segments”, the presence of the target RNA molecules can be amplified through the “releasable RNA segments” released in higher copies. The “releasable RNA segment” is variable and can be designed to comprise specific sequences. In some examples where the “releasable RNA segment” comprises the same sequence as the target RNA molecule, the target RNA molecule (or the sequence thereof) is amplified using the ribozyme of the present disclosure.

In one example, the ribozyme of the first aspect comprises one catalytic domain and one target binding domain, wherein the ribozyme comprises an RNA strand with the following structure:

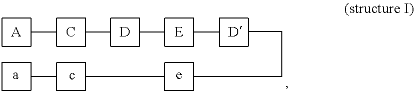

wherein [A] to [a] is in the 5′ to 3′ directionality or the 3′ to 5′ directionality, and wherein: motifs [A] and [a] constitute the target-binding domain for binding the target RNA molecule, motifs [C] and [c] constitute the catalytic domain, motif [D] comprises the first cleavage site capable of being cleaved by the catalytic domain motif [D′] comprises the second cleavage site capable of being cleaved by the catalytic domain, motif [E] comprises a releasable RNA segment, motif [e] comprises a sequence which is optionally complementary to the sequence of motif [E], wherein each of the horizontal lines connecting the motifs represents an optional linker region; and wherein the catalytic domain is in an active state when the target-binding domain is bound to the target RNA molecule.

In the above mentioned example, the binding of the target RNA molecule to the target-binding domain activates the catalytic domain, which results in the cleavage of both cleavage sites and the subsequent release of the releasable RNA segment.

In view of the definition of “complementary” provided earlier in the present description, it would be understood by a person skilled in the art that the expression “optionally complementary” as used herein and the present description encompasses not only full complementarity (100% complementary) and partially complementary (between 0% and 100% complementarity), but also non-complementarity (0% complementarity). In other words, for the ribozyme of the above mentioned example, having a region between motif [D′] and [c] that is at least partially complementary to motif [E] is an optional feature.

The term “directionality” as used herein refers to the end-to-end chemical orientation of a single strand of the RNA molecule. In a single strand of RNA, the chemical convention of naming carbon atoms in the nucleotide sugar-ring means that there will be a 5′-end, which contains a phosphate group attached to the 5′ carbon of the ribose ring, and a 3′-end which typically is unmodified from the ribose —OH substituent. As an illustrative example, when [A] to [A′] of strand S1 is in the 5′ to 3′ direction, [a] to [a′] of strand S2 will be in the 3′ to 5′ direction.

As used herein, the term “motif” refers to a region on an RNA strand that has a specific structure or is involved with a specific function. The term “domain” as used herein refers to a region of the ribozyme that has a specific structure involved with a specific function. As used herein, the term “domain” is used when referring to a structure formed by more than one RNA strand or by more than motifs of one RNA strand. As an illustrative example, the target-binding domain comprises both motifs [A] and [a], and the target-binding domain is considered “bound” to a target RNA molecule only when both motifs are bound to the RNA molecule.

In an example, the ribozyme as disclosed herein further comprises one or more inhibitory domains; wherein each of the one or more catalytic domains is functionally linked to one of the one or more inhibitory domains, wherein the catalytic domain is in an inactive state due to inhibition from the inhibitory domain, said inhibitory domain being linked to one of the one or more target-binding domains; wherein when one of the one or more target-binding domains is bound to the target RNA molecule, the inhibitory domain linked to said target-binding domain ceases to inhibit the catalytic domain linked to said inhibitory domain, which results in the catalytic domain switching to an active state. In this example, the linkage between a target-binding domain and a catalytic domain is achieved by an inhibitory domain, which is linked to both the target-binding domain and the catalytic domain.

In one example, the ribozyme comprises one catalytic domain, one inhibitory domain, and one target binding domain, wherein the ribozyme comprises an RNA strand with the following structure:

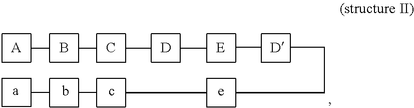

wherein [A] to [a] is in the 5′ to 3′ directionality or the 3′ to 5′ directionality, and
wherein: motifs [A] and [a] constitute the target-binding domain for binding the target RNA molecule, motifs [B] and [b] constitute the inhibitory domain, motifs [C] and [c] constitute the first catalytic domain, motif [D] comprises the first cleavage site capable of being cleaved by the catalytic domain, motif [D′] comprises the second cleavage site capable of being cleaved by the catalytic domain, motif [E] comprises a releasable RNA segment, motif [e] comprises a sequence which is optionally complementary to the sequence of motif [E], each of the horizontal lines connecting the motifs represents an optional linker region; and wherein the inhibitory domain is characterized by i) or ii) below:
   i) motif [b] anneals with [C] when the target-binding domain is not bound by the target RNA molecule, but anneals with [B] when the target-binding domain is bound by the target RNA molecule,
   ii) motif [B] anneals with motif [c] when the first target-binding domain is not bound by the target RNA molecule, but anneals with [b] when the target-binding domain is bound by the target RNA molecule, wherein the catalytic domain is in an active state when motif [b] is annealed with motif [B].

As is commonly known in the art, secondary structures are commonly formed within a ribozyme. In the examples of the present disclosure, one or more secondary structures are either formed individually by any of the motifs or the optional linker regions, or formed collectively by motifs, linker regions, or combinations thereof. In an example, the optional linker regions individually or collectively form one or more secondary structures.

As used herein, the term “secondary structure” refers to structures formed by the interactions between nucleotides in one or more polynucleotides. Examples for secondary structures include, but are not limited to, single-nucleotide bulges, three-nucleotide bulges, stems, stem loops, t-RNA type structures, cloverleaves, tetraloops, pseudoknots, symmetrical internal loops, asymmetrical internal loops, three stem junctions (3-way junctions), four stem junctions (4-way junction), two-stem junctions (2-way junctions) or coaxial stacks or combinations thereof. Specific examples of secondary structures include stems, stem loops, t-RNA type structures, cloverleaves, tetraloops, pseudoknots or combinations thereof. As used herein, the term “stem loop”, also known as a “hairpin loop”, refers to a secondary nucleic acid structure that forms when two regions of the same strand, usually complementary in nucleotide sequence when read in opposite directions, base-pair to form a double helix that ends with an unpaired loop.

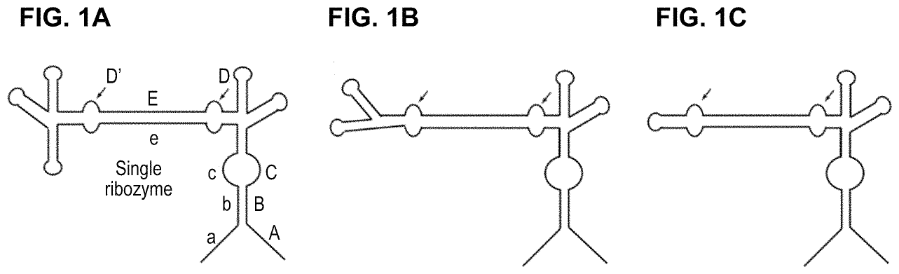

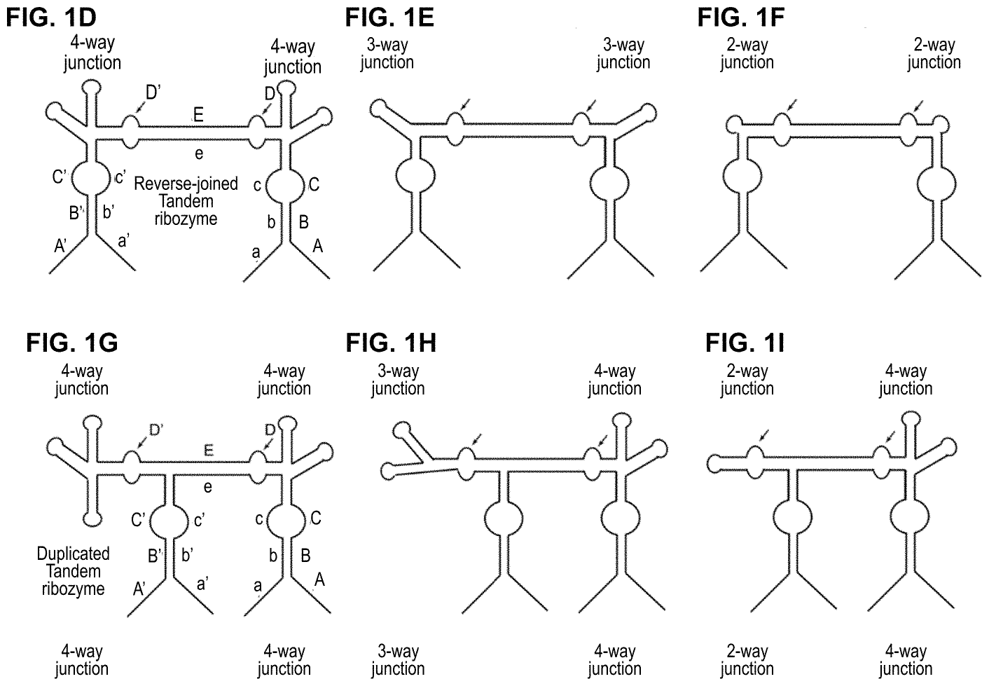

FIGS. 1A to 1L illustrate some exemplary and non-limiting structures formed by the ribozyme of the present disclosure. In particular, FIGS. 1A-1C provide examples of the ribozyme formed by one RNA strand and having one target-binding domain. The labeling of the motifs are consistent with the general structure provided above. In FIG. 1A, the ribozyme is characterized by two 4-way junctions formed on either side of the releasable RNA segment (motif [E]), with the catalytic domain (motifs [C] and [c]), optional inhibitory domain (motifs [B] and [b]), and target binding domain (motifs [A] and [a]) based on the extension of one of the helices of one of the 4-way junction structures. The ribozymes of FIGS. 1B and 1C adopt a similar structure to FIG. 1A, except that the 4-way junction formed by the region between motif [D] and motif [e] is replaced with a 3-way junction (FIG. 1B) or a 2-way junction (FIG. 1C). While not specifically illustrated, each of the 4-way junctions can also be a 3-way or a 2-way junctions and vice versa.

In examples where the ribozyme comprises one target binding domain, said target binding domain is comprised of motifs [A] and [a], motif [A] binds with a region of the target RNA molecule, and motif [a] binds with a second region of the target RNA molecule; wherein the target-binding domain is bound to the target RNA molecule when both [A] and [a] are bound to the target RNA molecule. Thus in a further example, motif [A] is complementary with a region of the target RNA molecule, and motif [a] is complementary with a second region of the target RNA molecule. In a further example, motif [A] is fully complementary with a region of the target RNA molecule, and motif [a] is fully complementary with a second region of the target RNA molecule. In an example, the first and second regions are adjacent to each other on the target RNA molecule.

In some examples, the ribozyme comprises two catalytic domains. In some examples, the ribozyme comprises two inhibitory domains. In some examples, the ribozyme comprises two target-binding domains. In a particular example, the ribozyme comprises two catalytic domains, each of the two catalytic domains is inhibited by one of the two inhibitory domains, wherein each of the inhibitory domains is further linked to one of the two target-binding domains.

As is commonly known in the art, a ribozyme can comprise one or more RNA strands. In a specific example, the ribozyme comprises a first RNA strand and a second RNA strand. When a ribozyme is comprised of two RNA strands, the two RNA strands have sufficient complementarity so that they are bound to each other. Typically, the two RNA strands are not fully complementary across their entire lengths. Each RNA strand can form secondary structures independently, as is generally known in the art. In one example, the ribozyme comprises the following structure:

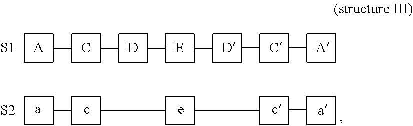

wherein: S1 is the first RNA strand and S2 is the second RNA strand, wherein [A] to [A′] and [a] to [a′] represent opposite directionalities; and motifs [A] and [a] constitute a first target-binding domain for binding a first target RNA molecule, motifs [C] and [c] constitute a first catalytic domain, motif [D] comprises a first cleavage site capable of being cleaved by the first catalytic domain, motifs [A′] and [a′] constitute a second target-binding domain for binding a second target RNA molecule, motifs [C′] and [c′] constitute a second catalytic domain, motif [D′] comprises a second cleavage site capable of being cleaved by the second catalytic domain, motif [E] comprises a releasable RNA segment, motif [e] comprises a sequence which is optionally complementary to the sequence of motif [E], each of the horizontal lines connecting the motifs represents an optional linker region; and wherein the first catalytic domain is in an active state when the first target-binding domain is bound to first target RNA molecule; and wherein the second catalytic domain is in an active state when the second target-binding domain is bound to the second target RNA molecule.

In another example, the ribozyme comprises the following structure:

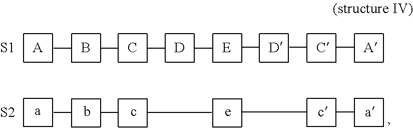

wherein: S1 is the first RNA strand and S2 is the second RNA strand, wherein [A] to [A′] and [a] to [a′] represent opposite directionalities; wherein motifs [A] and [a] constitute a first target-binding domain for binding a first target RNA molecule, motifs [B] and [b] constitute a first inhibitory domain, motifs [C] and [c] constitute a first catalytic domain, motif [D] comprises a first cleavage site capable of being cleaved by the first catalytic domain, motifs [A′] and [a′] constitute a second target-binding domain for binding a second target RNA molecule; motifs [C′] and [c′] constitute a second catalytic domain, motif [D′] comprises a second cleavage site capable of being cleaved by the second catalytic domain, motif [E] comprises a releasable RNA segment, motif [e] comprises a sequence which is optionally complementary to the sequence of motif [E], each of the horizontal lines connecting the motifs represents an optional linker region; wherein the first inhibitory domain is characterized by i) or ii) below:
i) motif [b] anneals with [C] when the first target-binding domain is not bound by the first target RNA molecule, but anneals with [B] when the first target-binding domain is bound by the first target RNA molecule,
ii) motif [B] anneals with motif [c] when the first target-binding domain is not bound by the first target RNA molecule, but anneals with [b] when the first target-binding domain is bound by the first target RNA molecule, wherein the first catalytic domain is in an active state when motif [b] is annealed with motif [B].

In a further example, the first inhibitory domain is further characterized by i) or ii) below: i) motif [b] is at least 50% complementary to motif [C], and at least 20% complementary to motif [B], and ii) motif [B] is at least 50% complementary to motif [c], and at least 20% complementary to motif [b].

In a further example, the first inhibitory domain is further characterized by i) or ii) below:
i) motif [b] is at least 60%, at least 70%, at least 80% or at least 90% complementary to motif [C], and at least 20% complementary to motif [B], or
ii) motif [B] is at least 60%, at least 70%, at least 80% or at least 90% complementary to motif [c], and at least 20% complementary to motif [b].

In another example, the ribozyme comprises the following structure:

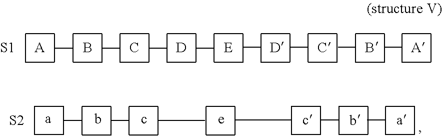

wherein:
S1 is the first RNA strand and S2 is the second RNA strand, wherein [A] to [A′] and [a] to [a′] represent opposite directionalities; wherein motifs [A] and [a] constitute a first target-binding domain for binding a first target RNA molecule, motifs [B] and [b] constitute a first inhibitory domain, motifs [C] and [c] constitute a first catalytic domain, motif [D] comprises a first cleavage site capable of being cleaved by the first catalytic domain, motifs [A′] and [a′] constitute a second target-binding domain for binding a second target RNA molecule; motifs [B′] and [b′] constitute a second inhibitory domain, motifs [C′] and [c′] constitute a second catalytic domain, motif [D′] comprises a second cleavage site capable of being cleaved by the second catalytic domain, motif [E] comprises a releasable RNA segment, motif [e] comprises a sequence which is optionally complementary to the sequence of motif [E], each of the horizontal lines connecting the motifs represents an optional linker region; wherein the first inhibitory domain is characterized by i) or ii) below:
i) motif [b] anneals with [C] when the first target-binding domain is not bound by the first target RNA molecule, but anneals with [B] when the first target-binding domain is bound by the first target RNA molecule,
ii) motif [B] anneals with motif [c] when the first target-binding domain is not bound by the first target RNA molecule, but anneals with [b] when the first target-binding domain is bound by the first target RNA molecule,

wherein the second inhibitory domain is characterized by i) or ii) below:
i) motif [b′] anneals with motif [C′] when the second target-binding domain is not bound by the first target RNA molecule, but anneals with [B′] when the second target-binding domain is bound by the second target RNA molecule,
ii) motif [B′] anneals with motif [c′] when the second target-binding domain is not bound by the second target RNA molecule, but anneals with [b′] when the second target-binding domain is bound by the second target RNA molecule,

wherein the first catalytic domain is in an active state when motif [b] is annealed with motif [B], and the second catalytic domain is in an active state when motif [b′] is annealed with motif [B′].

As mentioned earlier in the description, one or more secondary structures can be formed either individually by any of the motifs or optional linker regions, or formed collectively by motifs, linker regions, or combinations thereof. In an example, the optional linker regions individually or collectively form one or more secondary structures. In some examples of the ribozyme where the ribozymes comprise two target-binding domains and two catalytic domains, the ribozyme comprises one of the following structures:

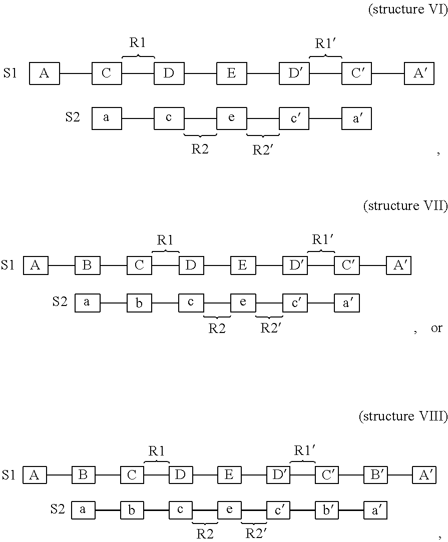

wherein linker regions R1 and R2 individually or collectively form one or more secondary structures, and linker regions R1′ and R2′ individually or collectively form one or more secondary structures.

In some examples, each of the linker regions R1, R2, R1′ and R2′ has a length independently selected from a length between 3-1000 nucleotides, 3-500 nucleotides, 3-300 nucleotides, 3-200 nucleotides, 3-100 nucleotides, 3-80 nucleotides, 3-70 nucleotides, 3-60 nucleotides, 3-50 nucleotides, 3-40 nucleotides, 3-30 nucleotides. In some specific examples, the lengths of linker regions R1 and R1′ are 40-100 nucleotides in length, 50-80 nucleotides in length, 50-70 nucleotides in length, or 60-70 nucleotides in length. In some specific examples, the lengths of linker regions R2 and R2′ are 10-60 nucleotides in length, 20-40 nucleotides in length, or 25-35 nucleotides.

The one or more secondary structures formed by any one of or any combinations of the linker regions R1, R2, R1′ and R2′ individually or collectively are independently selected and can be the same or different. In a specific example, linker regions R1 and R2 form a 2-way, 3-way, or 4-way junction. In a specific example, linker regions R1′ and R2′ form a 2-way, 3-way, or 4-way junction. The secondary structures formed by R1 and R2 are selected independently from the secondary structures formed by R1′ and R2′. To illustrate, R1 and R2 can form a 4-way junction while R1′ and R2′ form a 3-way junction. In one example, R1 and R2 form a 4-way junction, and that R1′ and R2′ also form a 4-way junction.

FIGS. 1D to 1I provide some examples of the ribozyme formed by two RNA strands and having two target-binding domains. The labeling of the motifs are consistent with the general structure provided above. The ribozyme of FIG. 1D is characterized by two 4-way junctions formed on either side of the releasable RNA segment, with each side comprising a catalytic domain, an optional inhibitory domain, and a target binding domain. FIGS. 1E and 1F adopt a similar structure to FIG. 1D, except that the 4-way junctions are replaced by 3-way junctions and 2-way junctions. The ribozyme of each of FIGS. 1D to 1F can be considered a tandem ribozyme reverse-joined by two “single ribozymes” (hence “reverse-joined tandem ribozyme”), with the two “single ribozymes” displaying “mirror-image” orientations. While the ribozyme of FIGS. 1G to 1I can also be considered a tandem ribozyme joined by two “single ribozymes”, the two “single ribozymes” adopt the same orientation (hence named “duplicated tandem ribozyme”). The ribozyme of FIG. 1G is characterized by two 4-way junctions formed on either side of the releasable RNA segment. The ribozyme of FIGS. 1H and 1I adopt a similar structure to FIG. 1G, except that one of the 4-way junctions is replaced by a 3-way junction (FIG. 1H) or by a 2-way junction (FIG. 1I).

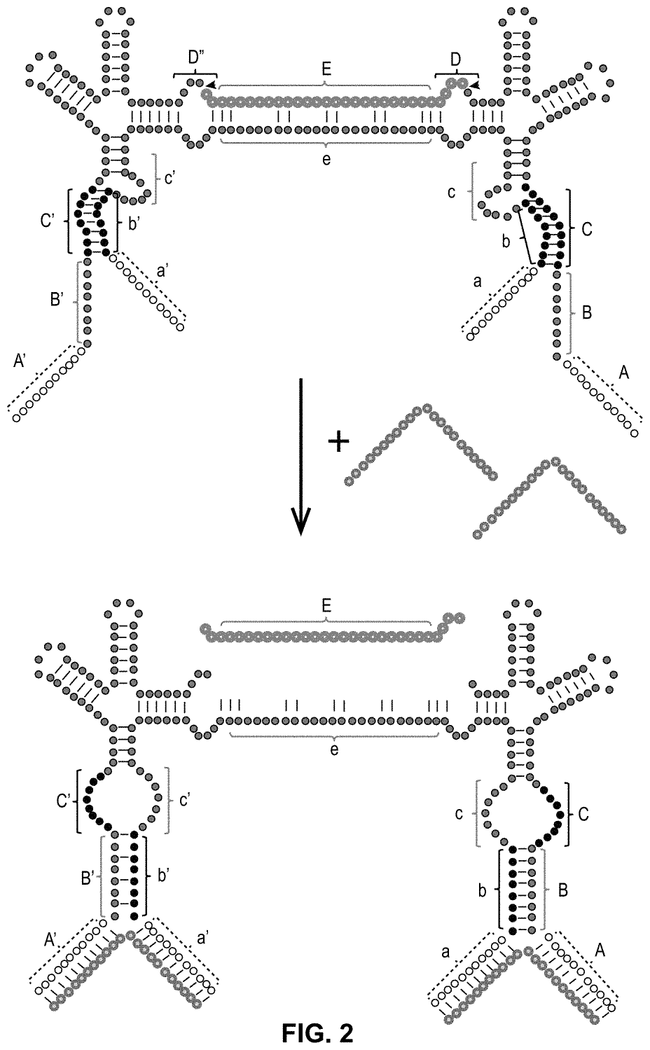

For an illustrative example on how the different motifs and domains are comprised on an exemplary ribozyme and function together, please refer to FIG. 2. FIG. 2 illustrates a specific configuration where the ribozyme has a reverse-joined tandem ribozyme structure. The variable motif [b] connecting to motif [c] (of the catalytic domain) to the motif [a] (of the target-binding domain) is designed to have complementarity to motif [C] of the catalytic domain. The variable motif [b] connecting to motif [c] (of the catalytic domain) to the motif [a] (of the target-binding domain) is designed to have complementarity to motif [C] of the catalytic domain. Without the target RNA molecule, the binding between [b] and [C] acts as a “toehold” to minimize self-cleavage, preventing cleavage of the cleavage site [D] and the release of the releasable RNA segment. target RNA binding activates cleavage, releasing the releasable RNA segment (the sequence of this product can be varied, which in some examples is loose complementarity to the ribozyme so that cleavage is favoured over re-ligation). The released target RNAs can bind anew to uncleaved ribozymes (present in excess) in a new cycle. As the ribozyme is in excess, a given copy number of target RNA molecules can activate self-cleavage of ribozymes, leading to the release of multiple cleavage products, hence amplifying the target RNA signal. It would be immediately understood by a person skilled in the art that the working principles illustrated herein apply mutatis mutandis to the ribozymes of other structures, such as those illustrated in FIGS. 1A-I.

In a further example, the first and second target RNA molecules are identical. In another example, the first and second target RNA molecules are different. The expression “the first and second target RNA molecules are identical” refers to the scenario where the ribozyme is activated (resulting in the release of the releasable RNA segment) by one specific target RNA molecule (with one copy of said target RNA molecule binding one of the two target-binding domains). The expression “the first and second target RNA molecules are different” refers to the scenario where the ribozyme is activated by two different target RNA molecules (each target-binding domain for binding one of the two target RNA molecules).

To illustrate and without being bound by theory, the active state of the first catalytic domain requires specific nucleotides in motifs [C] and [c] to form specific interactions with the cleavage site via a secondary structure formed by the catalytic domain. When either motif [C] or [c] is annealed by motif [b] or [B], the specific secondary structure necessary to support these intermolecular interactions with the cleavage site cannot be formed.

In a further example, the first inhibitory domain is further characterized by i) or ii) below:
i) motif [b] is at least 50% complementary to motif [C], and at least 20% complementary to motif [B], and
ii) motif [B] is at least 50% complementary to motif [c], and at least 20% complementary to motif [b];
   and the second inhibitory domain is further characterized by i) or ii) below:
i) motif [b′] is at least 50% complementary to motif [C′], and at least 20% complementary to motif [B′], and
ii) motif [B′] is at least 50% complementary to motif [c′], and at least 20% complementary to motif [b′].

In a further example, the first inhibitory domain is further characterized by i) or ii) below:
i) motif [b] is at least 80%, 90%, 95% or 100% complementary to motif [C], and at least 30% complementary to motif [B],
ii) motif [B] is at least 80%, 90%, 95% or 100% complementary to motif [c], and at least 30% complementary to motif [b];
   and the second inhibitory domain is further characterized by i) or ii) below:
i) motif [b′] is at least 80%, 90%, 95% or 100% complementary to motif [C′], and at least 30% complementary to motif [B′],
ii) motif [B′] is at least 80%, 90%, 95% or 100% complementary to motif [c′], and at least 30% complementary to motif [b′].

In some examples, motif [b] is fully complementary to motif [C], or motif [B] is fully complementary to motif [c]. In some examples, motif [b′] is fully complementary to motif [C′], or motif [B′] is fully complementary to motif [c′].

In some examples, the complementarity between motif [b] and motif [B] is 100%. In other examples, the complementarity between motif [b] and motif [B] is from 30% to 70%. In a specific example, the complementarity between motif [b] and motif [B] is about 50%.

In some examples where motifs [B′] and [b] are comprised on the ribozyme, the complementarity between motif [b′] and motif [B′] is 100%. In other specific examples, the complementarity between motif [b′] and motif [B′] is from 30% to 70%. In a specific example, the complementarity between motif [b′] and motif [B′] is about 50%.

In one example, when motif [b] is annealed to motif [B], or when motif [b′] is annealed to motif [B′], the annealing is based on alternating segments of complementarity and non-complementarity, wherein each segment is one nucleotide in length.

In some examples, wherein ribozyme comprises the structure of structure IV or VII, and wherein [A] to [A′] is in the 5′ to 3′ direction, motif [B] is partially complementary to motif [b]. In some examples, the ribozyme comprises the structure of V or VIII, with motif [B] partially complementary to motif [b], and motif [B′] fully complementary to motif [b′]. In some other examples, wherein the ribozyme comprises the structure of V or VIII, and wherein [A] to [A′] is in the 5′ to 3′ direction, with motif [B] fully complementary to motif [b], and motif [B′] fully complementary to motif [b′].

In some examples, each of motifs [B], [b], [C], [c], [B′], [b′], [C′], [c′] and [D] is independently between 1 to 100 nucleotides in length. In some specific examples, each of motifs [B], [b], [C], [c], [B′], [b′], [C′], [c′] and [D] is between 1 to 5, or between 5 to 10, or between 10 to 15, or between 15 to 20, or between 20 to 30, or between 30 to 40, or between 40 to 50, between 50 to 60, between 60 to 70, between 70 to 80 or 3, 4, 5, 6, 7, 8, 9, 10, 11, 12, 13, 14, 15, 16 nucleotides in length. In some specific examples, motif [B] has the same length as motif [b]. In some specific examples, motif [C] has the same length as motif [c]. In some specific examples, motif [B′] has the same length as motif [b′]. In some specific examples, motif [C′] has the same length as motif [c]. In one example, each of motifs [B], [b], [B′], [b′] is between 1-20 nucleotides in length. In another example, each of motifs [C], [c], [C′], and [c′] is between 5-12 nucleotides in length. In one example, each of motifs [B], [b], [B′], [b′] is 8 nucleotides in length. In another example, each of motifs [C], [c], [C′], and [c′] is 8 nucleotides in length.

In another example, motifs [A] and [A′] bind with a first region of the first and second target RNA molecule respectively, and motifs [a] and [a′] bind to a second region of the first and second target RNA molecule respectively; wherein the first target-binding domain is bound to the first target RNA molecule when both [A] and [a] are bound to the first target RNA molecule, and the second target-binding domain is bound to the second target RNA molecule when both [A′] and [a′] are bound to the second target RNA molecule. In a specific example, the first and second region on the first or second target RNA molecule are adjacent to each other. In a further example, the first and second region are equal in length. Thus in a further example, motif [A] is complementary with a first region of the first target RNA molecule, motif [a] is complementary with a second region of the first target RNA molecule, motif [A′] is complementary with a first region of the second target RNA molecule, motif [a′] is complementary with a second region of the second target RNA molecule. In a further example, motif [A] is fully complementary with a first region of the first target RNA molecule, motif [a] is fully complementary with a second region of the first target RNA molecule, motif [A′] is fully complementary with a first region of the second target RNA molecule, motif [a′] is fully complementary with a second region of the second target RNA molecule. In an example, the first and second region of first target RNA molecule are adjacent to each other. In an example, the first and second region of second target RNA molecule are adjacent to each other.

In some examples, the nucleotides of motifs [A] and [a] complementarily bind to the opposite ends of the target RNA molecule respectively. For example, motif [A] can complementarily bind to the 5′ end of the target RNA molecule, while motif [a] can complementarily bind to the 3′ end of the target RNA molecule, and vice versa. In some other examples, each of motifs [A] or [a] is between 1 to 5, or between 5 to 10, or between 10 to 15, or between 15 to 20, or between 20 to 30, or between 30 to 40, or between 40 to 50, between 50 to 60, between 60 to 70, between 70 to 80 nucleotides in length. In some examples, motif [a] is 11 nucleotides long. In some examples, the above descriptions for motifs [A] and [a] also apply to motifs [A′] and [a′].

The complementary binding is either partially complementary or fully complementary. For example, motif [A] is between about 70 to about 80%, or between about 80% to about 90%, or between about 90% to about 100%, or between about 75% to about 85%, or between about 85% to about 95%, or between about 95% to about 100%, or between about 88% to about 98%, or about 90%, about 91%, about 92%, about 93%, about 94%, about 95%, about 96%, about 97%, about 98%, about 99% or about 100% complementary to the 5′ end of the target RNA molecule; and motif [a] is between about 70 to about 80%, or between about 80% to about 90%, or between about 90% to about 100%, or between about 75% to about 85%, or between about 85% to about 95%, or between about 95% to about 100%, or between about 88% to about 98%, or about 90%, about 91%, about 92%, about 93%, about 94%, about 95%, about 96%, about 97%, about 98%, about 99% or about 100% complementary to the 3′ end of the same target RNA molecule, and vice versa. The lengths of motifs [A] and [a] are independent from each other, and can be the same or different. For example, each of motifs [A] and [a] can be between 3 to 5, or between 5 to 10, or between 10 to 15, or between 15 to 20, or between 20 to 30, or between 30 to 40, or between 40 to 50, or 3, 4, 5, 6, 7, 8, 9, 10, 11, 12, 13, 14, 15 nucleotides in length. In some specific examples, each of motifs [A] and [a] is 11 nucleotides long.

In some examples, motifs [E] and [e] is less than 70%, less than 60%, less than 50%, less than 40%, or less than 30% complementary to each other. In a specific example, the complementarity between motifs [E] and [e] is characterized by alternating regions of complementarity and regions of non-complementarity. In a particularly specific example, each region of complementarity is not more than 3 consecutive nucleotides in length, and each region of non-complementarity is at least 3 consecutive nucleotides in length. In some examples, each of motifs [E] and [e] is between 3 to 50 nucleotides in length. In some specific examples, each of motifs [E] and [e] is between 3 to 5, or between 5 to 10, or between 10 to 15, or between 15 to 20, or between 20 to 30, or between 30 to 40, or between 40 to 50, between 50 to 60, between 60 to 70, between 70 to 80 or 21, 22, 23, 24, 25, 26, 27, 28, 29, 30, 31, 32, 33, 34, 35, 36, 37, 38, 39, 40 nucleotides in length. In some specific examples, each of motifs [E] and [e] is 23 nucleotides in length.

As used herein, a “region of complementarity” refers to a region of the ribozyme in which the first and second RNA strand are fully complementary to each other; and a “region of non-complementarity” refers to a region of the ribozyme in which the first and second RNA strand are not complementary to each other.

In some examples, the ribozyme is modified from a naturally existing ribozyme. In some examples, the ribozyme is modified from an artificial ribozyme, fusion ribozyme, fragments and derivatives thereof. In a specific example, the ribozyme is characteristic of a hairpin ribozyme or a hammerhead ribozyme, or fragments and fusions thereof. In a specific example, the ribozyme is a twin ribozyme or duplex ribozyme. In a specific example, the ribozyme comprises a twin-hairpin ribozyme structure, as illustrated in FIG. 1.

In an example, wherein the ribozyme comprises the structure of structure V or VIII, the region between motif [A] and motif [E] (in the direction of [A] to [E]) on the first RNA strand (S1) comprises the sequence of SEQ ID NO.: 27).

SEQ ID NO.: 27 refers to the sequence:
5'-GUAUAUUAGUAUAUUACCUGGUUUUCGAUCGAAAGAUCGACGAGGU
GAAAACCUCGUGACAGUCC -3'.

In an example, wherein the ribozyme comprises the structure of structure III or VI, the region between motif [A] and motif [E] (in the direction of [A] to [E]) on the first RNA strand (S1) comprises the sequence of SEQ ID NO.: 81.

SEQ ID NO.: 81 refers to the sequence:
5'-GGGAAGUGGUAUAUUACCUGGUUUUCGAUCGAAAGAUCGACGAGGU
GAAAACCUCGUGACAGUCC -3'.

In an example, wherein the ribozyme comprises the structure of structure V or VIII, the region between motif [E] and motif [A′] (in the direction of [E] to [A′]) on the first RNA strand (S1) comprises the sequence of SEQ ID NO.: 28.

SEQ ID NO.: 28 refers to the sequence:
5'-CAGUCCUGAUUGUGCUCGAAAGAGCACAGACCUGAAAAGGUCUUUC
GUGGUAUAUUACUCUCUGAA -3'.

In an example, wherein the ribozyme comprises the structure of structure III or VI, the region between motif [E] and motif [A′] (in the direction of [E] to [A′]) on the first RNA strand (S1) comprises the sequence of SEQ ID NO.: 82.

SEQ ID NO.: 82 refers to the sequence:
5'-CAGUCCUGAUUGUGCUCGAAAGAGCACAGACCUGAAAAGGUCUUUC
GUGGUAUAUUACCUGGUCCC -3'.

In an example, wherein the ribozyme comprises the structure of structure V or VIII, the region between motif [a′] and motif [e] (in the direction of [a′] to [e]) on the second RNA strand (S2) comprises the sequence of SEQ ID NO.: 30.

SEQ ID NO.: 30 refers to the sequence:
5'-GUAAUAUAAGAAACACACGAAUCAGAGAAG -3'.

In an example, wherein the ribozyme comprises the structure of structure III or VI, the region between motif [a′] and motif [e] (in the direction of [a′] to [e]) on the second RNA strand (S2) comprises the sequence of SEQ ID NO.: 84.

SEQ ID NO.: 84 refers to the sequence:
5'- GGGACCAGAGAAACACACGAAUCAGAGAAG -3'.

In an example, wherein the ribozyme comprises the structure of structure V or VIII, the region between motif [e] and motif [a] (in the direction of [e] to [a]) on the first RNA strand (S1) comprises the sequence of SEQ ID NO.: 31.

SEQ ID NO.: 31 refers to the sequence:
5′-GAGAAGUCAACCAGAGAAACAUAAUAUAC-3′.

In an example, wherein the ribozyme comprises the structure of structure III or VI, the region between motif [e] and motif [a] (in the direction of [e] to [a]) on the first RNA strand (S1) comprises the sequence of SEQ ID NO.: 85).

SEQ ID NO.: 85 refers to the sequence:
5′-GAGAAGUCAACCAGAGAAACACACUUCCC-3′.

In an example, wherein the ribozyme comprises the structure of structure III or VI, the first RNA strand (S1) and second RNA strand (S2) comprise the sequences of SEQ ID NO.: 80 and SEQ ID NO.: 83 respectively.

SEQ ID NO.: 80 refers to the sequence:
5′-(n)ₐGGGAAGUGGUAUAUUACCUGGUUUUCGAUC
GAAAGAUCGACGAGGUGAAAACCUCGUGACAGUCC(n)ₓ
CAGUCCUGAUUGUGCUCGAAAGAGCACAGACCUGAAAA
GGUCUUUCGUGGUAUAUUACCUGGUCCC (n)_{b}-3′,

wherein (n)ₓ represents a region partially complementary to the releasable RNA segment on S1, and (n)ₐ and (n)_{b} represent sequences complementary to the second and first target RNA molecules.

SEQ ID NO.: 83 refers to the sequence:
5′-(n)ₐGGGACCAGAGAAACACACGAAUCAGAGAAG(n)ₓG
AGAAGUCAACCAGAGAAACACACUUCCC(n)_{b}-3′,

wherein (n)ₓ represents a region partially complementary to the releasable RNA segment on S1, and (n)ₐ and (n)_{b} represent sequences complementary to the second and first target RNA molecules.

In another example, wherein the ribozyme comprises the structure of structure V or VIII, the first RNA strand (S1) and second RNA strand (S2) comprise the sequences of SEQ ID NO.: 26 and SEQ ID NO.: 29 respectively.

SEQ ID NO.: 26 refers to the sequence:
5′-(n)ₐGUAUAUUAGUAUAUUACCUGGUUUUCGAUCGAAA
GAUCGACGAGGUGAAAACCUCGUGACAGUCC(n)ₓCAGUCC
UGAUUGUGCUCGAAAGAGCACAGACCUGAAAAGGUCUUUCG
UGGUAUAUUACUCUCUGAA(n)_{b}-3′,

wherein (n)ₐ corresponds to a variable sequence corresponding to motif [A] of the first target-binding domain, (n)ₓ corresponds to a variable sequence corresponding to motif [E], and (n)_{b} corresponds to a variable sequence corresponding to motif [A′] of the second target-binding domain.

SEQ ID NO: 29 refers to the sequence:
5′-(n)ₐGUAAUAUAAGAAACACACGAAUCAGAGAAG
(n)ₓGAGAAGUCAACCAGAGAAACAUAAUAUAC(n)_{b}-3′,

wherein (n)ₐ corresponds to a variable sequence corresponding to motif [a′] of the second target-binding domain, (n)ₓ corresponds to a variable sequence corresponding to motif [e], and (n)_{b} corresponds to a variable sequence corresponding to motif [a] of the second target-binding domain.

The ribozymes can be in the single-catalytic domain configuration. These ribozymes comprises only 1 RNA strand, as resembled by structures I and II.

A general sequence for the single-catalytic domain ribozymes, wherein the ribozyme comprises the structure of structure II, is provided below:

(SEQ ID.: 88)
5′-(n)ₐGUAUAUUAGUAUAUUACCUGGUUUUCGAUCGAA
AGAUCGACGAGGUGAAAACCUCGUGACAGUCC(n)ₓCAGU
CCUGAUUGUGAUCGAAAGAGCACAGACCUGAAAAGGUCUU
UCUGGUUUCGACCAGAAUCAGAGAAG(n)_{y}GAGAAGUCAA
CCAGAGAAACAUAAUAUAC(n)_{b}-3′,

wherein (n)ₐ and (n)_{b} represent sequences complementary to the first and second target region of the RNA; (n)ₓ represents a releasable RNA segment, and (n)_{y} represents a sequence complementary to the releasable RNA segment.

In some examples, the number of nucleotides in each of the variable sequences listed above is independently a number between 3 to 150, 3 to 100, 3 to 80, 3 to 60, 6 to 50, 6 to 40, 6 to 30, 10-15, or 15 to 23.

In a specific example, the sequence of the releasable RNA segment (or motif [E] in some examples) comprises the sequence of 5′-GUCCUUAGUCGAAAGUUUUACUAGAGUCA-3′ (SEQ ID NO.: 25).

In an example, wherein the ribozyme comprises the structure of any of the structures III-VIII, the first RNA strand (S1) and second RNA strand (S2) comprise the sequences of SEQ ID NO.: 34 and SEQ ID NO.: 35 respectively. In another example wherein the ribozyme comprises the structure of structure V or VIII, the first RNA strand (S1) and second RNA strand (S2) comprise the sequences of SEQ ID NO.: 32 and SEQ ID NO.: 33 respectively.

In an example, the target RNA molecule is more than 16, more than 18, more than 20, more than 22, more than 24, more than 26, more than 28, more than 30, more than 32, more than 34, more than 36, more than 38, more than 40 nucleotides, more than 50 nucleotides, more than 60 nucleotides, more than 70 nucleotides, more than 80 nucleotides, more than 90 nucleotides, or more than 100 nucleotides in length. In a specific example, the target RNA molecule is 6 to 200, 6 to 100, 6 to 80, 10-60, 10 to 30, 10 to 25, or 15 to 23 nucleotides in length. In yet another specific example, the target RNA is about 18-23 nucleotides in length.

In another example, the target RNA molecule comprises a region which is complementary to the target-binding domain, wherein said region is more than 16, more than 18, more than 20, more than 22, more than 24, more than 26, more than 28, more than 30, more than 32, more than 34, more than 36, more than 38, more than 40, more than 50 nucleotides, more than 60 nucleotides, more than 70 nucleotides in length. In a more specific example, the region is 6 to 100, 6 to 80, 6 to 30, 10 to 25, or 15 to 23 nucleotides in length. In yet another specific example, the region is about 6-80 nucleotides in length.

In another example, the releasable RNA segment is 3-200 nucleotides in length. In further examples, the releasable RNA segment is 3-150, or 6-120, or 6-100, or 6-80, or 6-60, or 10-50, or 20-40 nucleotides in length.

In some examples, wherein the ribozyme of the first aspect comprises two RNA strands S1 and S2, S1 and S2 comprise the sequences of SEQ ID NO. 2 and 3 respectively, or the sequences of SEQ ID NO. 5 and 6 respectively, the sequences of SEQ ID NO. 41 and 42 respectively, the sequences of SEQ ID NO. 44 and 45 respectively, the sequences of SEQ ID NO. 47 and 48 respectively, the sequences of SEQ ID NO. 70 and 71 respectively, the sequences of SEQ ID NO. 72 and 73 respectively, the sequences of SEQ ID NO. 74 and 75 respectively, the sequences of SEQ ID NO. 76 and 77 respectively, the sequences of SEQ ID NO. 78 and 79 respectively, the sequences of SEQ ID NO. 92 and 93 respectively, the sequences of SEQ ID NO. 94 and 95 respectively, the sequences of SEQ ID NO. 96 and 97 respectively, the sequences of SEQ ID NO. 98 and 99 respectively, the sequences of SEQ ID NO. 100 and 101 respectively. In some examples, wherein the ribozyme of the first aspect comprises one RNA strand, the ribozyme comprises the sequence of SEQ ID NO. 86 or 87.

In one example, the one or more target-binding domains of the ribozyme binds or is for binding the same target RNA molecule. In a specific example, the ribozyme comprises a first target-binding domain and a second target-binding domain, and the two target-binding domains are for binding the same target RNA molecule. As used herein, the “same target RNA molecule” refers to target RNA molecules with identical sequences.

In another example, the releasable RNA segment comprises a sequence that is identical to at least one of the one or more target RNA molecules. In a specific example, the ribozyme comprises two target-binding domains for binding a specific target RNA molecule, wherein the target RNA molecule comprises a sequence that is identical to the target RNA molecule. In this specific example, the binding of target RNA molecules leads to the release of an RNA molecule comprising the same sequence as the target RNA molecule.

In some examples, the binding of the target RNA molecule with the one or more target-binding domains of the ribozyme occurs at a temperature T1. In some specific examples, T1 is a temperature not more than 50° C. In other examples, T1 is a temperature between 0° C. to 50° C., a temperature between 15° C. to 45° C. a temperature between 25° C. to 45° C., a temperature between 30° C. to 40° C., a temperature between 35° C. to 38° C., a temperature between 36.5° C. to 37.5° C. In a specific example, T1 is a temperature of about 37° C.

In another example, a target RNA molecule bound to a target-binding domain of the ribozyme is released from said target-binding domain at a temperature T2. In some examples, wherein the two cleavage sites flanking the releasable RNA segment are cleaved, the releasable RNA segment is released at a preferred temperature T2. In some examples, T2 is a temperature between 20° C. to 100° C., a temperature between 25° C. to 80° C., a temperature between 30° C. to 80° C., a temperature between 35° C. to 80° C., a temperature between 40° C. to 80° C., a temperature between 45° C. to 80° C., a temperature between 50° C. to 80° C., a temperature between 55° C. to 75° C., or a temperature between 57° C. to 63° C. In a specific example, T2 is a temperature of about 60° C.

It should be understood that T1 and T2 as described above refer to temperatures which allow the binding (of the targeting RNA molecule) and the release (of the target RNA molecules and the releasable RNA segment) respectively. T1 should not be taken to mean a temperature under which no target RNA molecules or releasable RNA segments can be released; and T2 should not be taken to mean a temperature under which no target RNA molecule can bind with the target-binding domain. It is generally understood in the art that the binding (also known as “annealing”) and release (also known as “melting”) of complementary RNA strands can occur simultaneously, albeit with differing kinetics, across a wide range of temperatures, therefore T1 and T2 can be the same or different. In some examples, the target RNA molecules can bind to the target-binding domains of the ribozyme, triggering the cleavage and release of the releasable RNA segment, and release from the ribozyme, all at a temperature between 35° C. to 38° C. However, when the ribozyme is used to detect and amplify target molecules, the implementation of annealing (under T1) and melting (under T2), wherein T2 is higher than T1, drives the reaction forward and results in increased number of released releasable RNA products.

In specific examples, the target RNA molecule is an RNA molecule obtained from animals, viruses, bacteria, yeast or plants.

In a specific example, the target RNA molecule is a genome of an RNA virus, or a fragment thereof. In further examples, the RNA virus is a single stranded or double stranded RNA virus. In further embodiments, the RNA virus is a positive sense RNA virus or a negative sense RNA virus or an ambisense RNA virus. In further examples, the virus is a Retroviridae virus, Lentiviridae virus, Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus.

In particular example, the RNA virus is selected from the group consisting of Lymphocytic choriomeningitis virus, Coronavirus, human immunodeficiency virus (HIV), Severe acute respiratory syndrome virus (SARS), Severe acute respiratory syndrome coronavirus 2 (SARS-CoV-2), Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza and Hepatitis D virus. In a particular example, the RNA virus is a Severe acute respiratory syndrome coronavirus 2 (SARS-CoV-2) virus.

In some examples, the target RNA molecule is a microRNA (miRNA), short interfering RNA (siRNA), small RNA (sRNA), messenger RNA (mRNA), non-coding RNA (ncRNA), short non-coding RNA, transfer RNA (tRNA), ribsomal RNA (rRNA), transfer-messenger RNA (tmRNA), clustered regularly interspaced short palindromic repeats RNA (CRIPSR RNA), antisense RNA, pre-mRNA or pre-miRNA, or fragment thereof. In a specific example, the target RNA molecule is a micro-RNA, or a precursor thereof, or a fragment thereof.

The term “micro-RNA” (abbreviated miRNA) as used herein refers to a small non-coding RNA molecule. It generally functions in RNA silencing and post-transcription regulation of gene expression. While the majority of miRNAs are located within the cell, some miRNAs, commonly known as circulating miRNAs or extracellular miRNAs, have also been found in extracellular environment, including various biological fluids and cell culture media. The length of the miRNAs can be between 18-25 nt long.

The ribozyme of the first aspect can be used for detecting presence of a target RNA molecule in a sample. Thus, in a second aspect, the present invention provides a method of detecting presence of a target RNA molecule in a sample, wherein the method comprises: a) incubating the sample with an ribozyme of the first aspect at temperature T1 which allows the binding of the target RNA molecule for binding with one or more target-binding domains comprised in the ribozyme; b) incubating the sample at temperature T2 which allows the target RNA molecule and the releasable RNA segment to be released from the ribozyme; c) detecting the release of the releasable RNA segment from the ribozyme. In one example, step c) is carried out by detecting the presence of the releasable RNA segment in the sample. Any RNA detection methods or RNA detection systems known in the art can be used. Exemplary and non-exhaustive examples of RNA detection methods include: Reverse transcription polymerase chain reaction (RT-PCR), quantitative RT-PCR (RT-qPCR), probe-based RNA detection (such as northern blotting, microarrays and molecular beacons). Exemplary and non-exhaustive examples of RNA detection systems include: NanoString Technologies' nCounter© miRNA expression assay and Exiqon's Smart Flares), RNA-activated fluorescent sensors such as the Pandan fluorescent sensor (PCT patent PCT/SG2017/050086; Aw et. al., Nucleic Acids Research 2016), and CRISPR-Cas based nucleic acid detection systems such as DETECTR (Chen, J. S. et al. CRISPR-Cas12a target-binding unleashes indiscriminate single-stranded DNase activity. Science 360, 436-439, doi:10.1126/science.aar6245 (2018)) and SHERLOCK (Gootenberg, J. S. et al. Multiplexed and portable nucleic acid detection platform with Cas13, Cas12a, and Csm6. Science 360, 439-444, doi:10.1126/science.aaq0179 (2018)).

In a specific example, steps a) to b) are repeated for one or more times before step c) is carried out, wherein the RNA molecules released from step b) are for binding to another copy of the ribozyme. In this example, the sample can be incubated with an excess amount of the ribozyme when performing step a) for the first time, so that when performing a) for the second or subsequent time, additional ribozymes may not be supplemented to the sample. As the target RNA molecules released from the ribozymes in step b) can further bind to new ribozymes, the copy number of released cleavage products (the “releasable RNA segment”) can be many folds higher than the copy number of target molecules in the sample. Therefore, the presence or the amount of the released “releasable RNA segment” serve as an amplified signal for the presence or the amount of the target RNA molecules in the sample. Thus, this method can improve the sensitivity of existing RNA detection technologies when used in combination.

The releasable RNA segment can comprise a sequence identical to the target RNA, in which case the released “releasable RNA segments” can further bind to new ribozymes as target RNA molecules themselves. By repeating steps a) to b) as above, exponential amplification of the target RNA molecule (or the sequence the target RNA molecule) can be achieved. Thus, in a third aspect, there is provided a method of amplifying a target RNA molecule, wherein the method comprises: a) incubating the target RNA molecule with an ribozyme of the first aspect at temperature T1 which allows the binding of the target RNA molecule with one or more target-binding domains comprised in the ribozyme, and wherein the releasable RNA segment comprises a sequence identical to the target RNA molecule; b) incubating the ribozyme bound to the target RNA molecule at temperature T2 which allows the target RNA molecule and the RNA segment to be released from the ribozyme. In a further example, steps a) to b) are repeated for one or more times, wherein the target RNA molecules and the releasable RNA segment released from step b) are for binding to another copy of the ribozyme.

Many RNA molecules, such as microRNAs serve as biomarkers useful in the diagnosis of diseases. The detection of genomic nucleic acids of RNA viruses is also useful in diagnosing viral infections and diseases caused by viruses. The ribozyme of the present invention can be conveniently modified to bind to RNA biomarkers indicated in diseases or viral RNAs. This can be achieved by modifying the sequences of the target-binding domains so that they are complementary to a specific region on the target RNA molecules of interest using ordinary skills in the art or by direct synthesis. Thus, in a fourth aspect, there is provided a method of diagnosing a disease in a subject, the method comprising: a) obtaining a sample from the subject; b) incubating the sample with the ribozyme according to the first aspect at temperature T1 which allows the binding of a disease associated target RNA molecule with one or more target-binding domains comprised in the ribozyme; c) incubating the sample at temperature T2 which allows the target RNA molecule and the releasable RNA segment to be released from the ribozyme; d) detecting the levels of the releasable RNA segment from the ribozyme.

The term “subject” as used herein can refer to any organisms. In some examples, the subject can be an animal subject or more specifically a human subject. In other examples, the subject can be a plant or a fungus. The term “disease” thus encompasses human diseases, animal diseases, plant diseases or fungal diseases.

The term “sample” as used herein refers to a sample suspected of containing the target RNA molecule. It may comprise a bodily fluid. The term “sample” used herein refers to a biological sample, or a sample that comprises at least some biological materials such as nucleic acids. The biological samples of this disclosure may be any sample suspected to contain the target nucleic acid sequence, including liquid samples from an animal or human subject, such as whole blood, blood serum, blood plasma, cerebrospinal fluid, central spinal fluid, lymph fluid, cystic fluid, sputum, stool, pleural effusion, mucus, pleural fluid, ascitic fluid, amniotic fluid, peritoneal fluid, saliva, bronchial washes, urine and other bodily fluid, or extracts thereof. The biological samples of this disclosure may also be obtained from a non-animal subject, for example a plant, a fungus or a bacterium.

In a specific example, the method further comprises comparing the levels of the releasable RNA segment with a control sample. As used herein, the term “control sample” refers to a sample obtained from a healthy subject, which has been incubated with the same ribozyme according to steps b) and c).

In some examples, T1 is a temperature not more than 50° C. In other examples, T1 is a temperature between 0° C. to 50° C., a temperature between 15° C. to 45° C. a temperature between 25° C. to 45° C., a temperature between 30° C. to 40° C., a temperature between 35° C. to 38° C., a temperature between 36.5° C. to 37.5° C. In a specific example, T1 is a temperature of about 37° C.

In some examples, T2 is a temperature between 20° C. to 100° C., a temperature between 25° C. to 80° C., a temperature between 30° C. to 80° C., a temperature between 35° C. to 80° C., a temperature between 40° C. to 80° C., a temperature between 45° C. to 80° C., a temperature between 50° C. to 80° C., a temperature between 55° C. to 75° C., or a temperature between 57° C. to 63° C. In a specific example, T2 is a temperature of about 60° C.

As mentioned earlier in the present description, as target-binding, ribozyme cleavage and target and product release, can occur simultaneously across a wide range of temperatures, the detection and amplification of target RNA molecules is possible with T1 and T2 being the same or different. However, the implementation of annealing (under T1) and melting (under T2) cycles, wherein T2 is higher than T1, helps to drive the reaction forward and results in increased number of released releasable RNA products. Therefore, in some examples, T2 is a temperature higher than T1. In some examples, the release of target molecules occurs at both T1 and T2. In an example, T1 is a temperature between 25° C. to 45° C., and T2 is a temperature between 51° C. to 80° C.

In a fifth aspect, there is provided a polynucleotide encoding the ribozyme of the first aspect of the disclosure. In some examples where the ribozyme is comprised of more than one RNA strands, the RNA strands can be encoded together on one polynucleotide or separately on several polynucleotides.

As used herein, the terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

In a sixth aspect, there is further provided a kit comprising the ribozyme of the first aspect of the disclosure.

In a specific example, the kit further comprises an RNA detection system. In some examples, the RNA detection system comprises an RNA-activated fluorescent sensor. In one specific example, the sensor is a Pandan fluorescent sensor or detection system (PCT patent PCT/SG2017/050086; Aw et. al., Nucleic Acids Research 2016). In a further example the RNA detection system is a CRISPR-Cas based nucleic acid detection system. CRISPR Cas-based RNA detection methods and systems are known in the art, and are disclosed for example in Chen, J. S. et al. CRISPR-Cas12a target-binding unleashes indiscriminate single-stranded DNase activity. Science 360, 436-439, doi:10.1126/science.aar6245 (2018), and Gootenberg, J. S. et al. Multiplexed and portable nucleic acid detection platform with Cas13, Cas12a, and Csm6. Science 360, 439-444, doi:10.1126/science.aaq0179 (2018).

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples.

### Experimental Section

detection platform with Cas13, Cas12a, and Csm6. Science 360, 439-444, doi:10.1126/science.aaq0179 (2018).

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### 1. Methods for Ribozyme Generation and Cleavage Experiments

### PCR Amplification of Template

PCR amplification of templates for in vitro transcription of RNA strands used in the cleavage reactions was performed using Phusion High Fidelity PCR Master Mix with HF Buffer (Cat #F531L; Thermo Fisher Scientific, USA) according to the manufacturer's instructions. Primers and templates used were designed as shown above and in the accompanying Sequence Listing Table. Resultant PCR products were purified using QIAquick PCR purification kit (Cat #28106; Qiagen, USA) according to manufacturer's protocols.

### In Vitro Transcription of RNA

Purified PCR products used as templates for in vitro transcription using Epicentre AmpliScribe™ T7-Flash™ Transcription Kit (Cat #ASF3507 Lucigen, USA) according to manufacturer's protocols. After the reaction, 280 μL of RNase-free water (Cat #SH30538.02; Hyclone, USA) was added to 20 μL of the in vitro transcription reaction. The RNA was purified by adding 300 μL of acid-phenol:chloroform, pH 4.5 (with IAA, 125:24:1) (Cat #AM9722 Invitrogen, USA), mixed, and centrifuged at 14,000 rpm for 3 min at room temperature. The aqueous phase was transferred to a new tube, and 300 μL of chloroform was added. The mixture was again centrifuged at 14,000 rpm for 3 min at room temperature. The aqueous phase was transferred to a new tube and the RNA was precipitated with 1/10 volume (25 μL) of 3 M sodium acetate at pH 5.2 and 2.5× volume (625 μL) of 100% ethanol. After overnight precipitation at −20° C., the samples were centrifuged at 13,000 rpm for 20 min at 4° C. The supernatant was discarded, the RNA pellet was washed with cold 75% ethanol, and centrifuged again at 13,000 rpm for 20 mins at 4° C. After centrifugation, the supernatant was removed completely and the pellet allowed to air-dry for approximately 30 sec. The RNA pellet was re-suspended in 50 μL of RNase-free water, and its concentration was measured using a NanoDrop spectrophotometer (Thermo Fisher Scientific, USA).

### Cleavage Reaction

Annealing and cleavage reactions were set up with 200 nM each of S1 and S2, with addition of either water (control) or 50 nM (unless otherwise stated) of the respective target RNA. Reactions were performed in reaction buffer (50 mM Tris, 12 mM magnesium chloride, and 5 mM spermine tetrahydrochloride, pH 7.4) in a total volume of 600 μL. The 600 μL reaction volume was divided into 6 aliquots of 100 μL each and pipetted into PCR strip tubes. Samples were incubated in a thermocycler with the following programme:

### 1.60° C. for 1 min

### 2. 56.5° C. for 1 min

### 3. 53° C. for 1 min

### 4. 49.5° C. for 1 min

### 5. 46° C. for 1 min

### 6. 42.5° C. for 1 min

### 7. 39° C. for 1 min

### 8. 37° C. for 15 min

9. Cycle from steps 1 to 8 for 9 times

### RNA Precipitation for Visualisation

At the end of the cleavage reaction, each sample was transferred to a 15 mL centrifuge tube, and terminated with the addition of 900 μL of Stop Buffer (7 M Urea and 50 mM EDTA). RNA was purified by addition of 1.8 mL of Acid-Phenol:Chloroform, pH 4.5 (with IAA, 125:24:1) (Cat #AM9722 Invitrogen, USA). Each sample was mixed and centrifuged at 13,000 rpm for 15 min at 4° C. The aqueous phase was transferred to a new tube, and 1.8 mL of chloroform (Cat #07278-00 Kanto Chemical, Japan) was added. Each sample was again mixed and centrifuged at 13,000 rpm for 15 min at 4° C. The aqueous phase was transferred to a new tube, and precipitated with 1/10 volume (120 μL) of 3 M sodium acetate at pH 5.2, 2.5× volume (3.3 mL) of 100% ethanol, 2 μL of RNA grade glycogen (Cat #R0551; Thermo Fisher Scientific, USA), and 1/100 volume (43 μL) of 1 M magnesium chloride. After overnight precipitation at −20° C., the samples were centrifuged at 13,000 rpm for 1 hour at 4° C. The supernatant was discarded, the RNA pellet was washed with cold 75% ethanol, transferred to a 1.5 mL microcentrifuge tube, and centrifuged again at 13,000 rpm for 30 mins at 4° C. After centrifugation, the supernatant was removed completely and the pellet allowed to air-dry for approximately 10 sec. The RNA pellet was re-suspended in 10 μL of RNase-free water, and its concentration was measured using NanoDrop spectrophotometer (Thermo Fisher Scientific).

300 μg of RNA was heat-denatured for 10 min at 70° C., loaded with RNA Loading Dye (Cat #B0363S NEB, USA) and separated on a 10% denaturing polyacrylamide gel (Cat #EC-833 National Diagnostics, USA) in 10×TBE (Tris/Boric Acid/EDTA) Buffer (Cat #1610770 Bio-Rad, USA) at 200 V for 1 hour, or until the dye front migrated to the bottom of the gel. Low range ssRNA ladder (Cat #N0364S NEB, USA) was loaded as a size marker, and 25 ng of a 29 nt oligo (5′-GUCCUUAGUCGAAAGUUUUACUAGAGUCA-3′) (IDT) (SEQ ID NO. 25), corresponding to the size of the expected cleavage product, was spiked in to the ladder as an additional size marker. Where appropriate, 25 ng of the target sequence was also spiked in to the ladder as a size marker. Gels were stained with 1:10,000 SYBR™ Gold Nucleic Acid Gel Stain (Cat #S11494 Invitrogen, USA), and visualized using Gel Doc XR+ gel documentation system (Bio-Rad, USA). Images were analyzed using Image Lab software (Bio-Rad, USA).

### Sensitivity Test

The sensitivity of ribozyme detection was tested using the Tlet7f_Cban3pR ribozyme, which detects hsa-let-7f-5p. Serial dilutions of the target miRNA were carried out. These were then added to cleavage reactions at final working concentrations of 50 nM, 10 nM, and 0.1 nM. Each cleavage reaction contained 200 nM of ribozyme for hsa-let-7f-5p.

### Specificity Test

Sequence specificity of the Tlet7f_Cban3pR ribozyme was tested by: 1) Adding a miRNA with a dissimilar sequence (hsa-miR-122-5p: 5′-UGGAGUGUGACAAUGGUGUUUG-3′) (SEQ ID NO. 24); 2) Adding equal concentrations of the two miRNAs (hsa-let-7f-5p and hsa-miR-122-5p), or 3) Spiking into the RNA mixture total extracted RNA from adult Drosophila melanogaster at a range of concentrations (10-fold, 100-fold, or 1000-fold total RNA compared to the amount of target miRNA). Each cleavage reaction contained 200 nM of ribozyme for hsa-let-7f-5p.

2. Sequences Used in the Ribozyme Design (with Two Target-Binding Domain Configuration)

The ribozyme with two target-binding domains (e.g. those resembled by structures III-V in the detailed description) is made up of two strands of RNA: Strand S1 (containing the RNA cleavage product) and Strand S2 (this strand will not be cleaved). To generate S1 and S2 RNA, we carried out in vitro RNA transcription using DNA templates generated through PCR. DNA templates and primers were ordered from Integrated DNA technologies (IDT). Forward primers to amplify S1 and S2 strands were designed to include a 5′ promoter sequence for T7 RNA polymerase (5′-GTATAATACGACTCACTATAGGGA-3′) (SEQ ID NO.: 7); the 3′ terminal GGGA will be included in the final transcribed RNA and be included at its 5′ end. However, the GGGA is not a functional part of the S1 or S2. Forward and reverse primers used to amplify S1 and S2 were designed to encode complementary sequences to the 3′ and 5′ region of the target RNA molecules, respectively. Examples for ribozymes targeting microRNAs are shown first, followed by examples for ribozymes targeting viral RNA sequences.

A. Examples of Ribozymes Targeting microRNAs Dme-Ban-5p (Tban5p Cban3pR) and Hsa-Let-7f (Tlet7f-Cban3pR), Both Cleaving Out Dme-ban3p-Reverse Sequence).

TABLE 1
Examples of ribozymes targeting microRNAs dme- ban-5p (Tban5p Cban3pR) and
hsa-let-7f (Tlet7f-Cban3pR), both cleaving out dme- ban3p-reverse sequence). The
underlined ( ) sequences represent sequences comprised on target-binding domains.

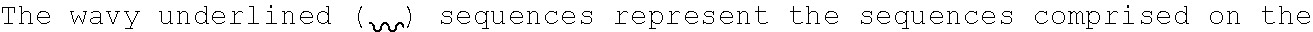

releasable RNA segment, or the sequences comprised on the region opposing and
at least partially complementary to the releasable RNA segment.

| SEQ | | | Name of ribozyme |
|---|---|---|---|
| ID | Name of RNA or | | formed by S1 and |
| NO. | sequence | Sequence of RNA (5′ to 3′) | S2 |
| 1 | Target RNA | CCGGUUUUCGAUUUGGUUUGACU | Tban5p_Cban3pR |
| | dme-bantam-5p | (SEQ ID NO 1) | |
| 2 | S1_Tban5p_ | GGGAAGUCAAACCAAGUAUAUUAG | |
| | Cban3pR | UAUAUUACCUGGUUUUCGAUCGAA | |
| | | AGAUCGACGAGGUGAAAACCUCGU | |
| | | 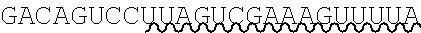 | |
| | | 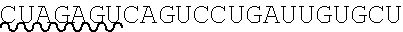 | |
| | | CGAAAGAGCACAGACCUGAAAAGG | |
| | | UCUUUCGUGGUAUAUUACUCUCUG | |
| | | AAAUCGAAAACCGG (SEQ ID NO 2) | |
| 3 | S2_Tban5p_ | GGGAAGUCAAACCAAGUAAUAUAA | |
| | Cban3pR | 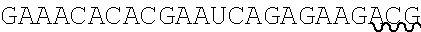 | |
| | | 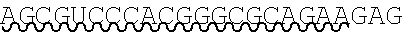 | |
| | | AAGUCAACCAGAGAAACAUAAUAUA | |
| | | CAUCGAAAACCGG (SEQ ID NO 3) | |
| 4 | Target RNA | UGAGGUAGUAGAUUGUAUAGUU | Tlet7f_Cban3pR |
| | hsa-let-7f-5p | (SEQ ID NO 4) | |
| 5 | S1_Tlet7f_Cban | GGGAAACUAUACAAUGUAUAUUAG | |
| | 3pR | UAUAUUACCUGGUUUUCGAUCGAA | |
| | | AGAUCGACGAGGUGAAAACCUCGU | |
| | | 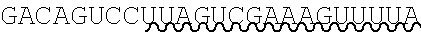 | |
| | | 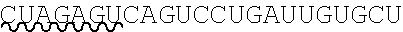 | |
| | | CGAAAGAGCACAGACCUGAAAAGG | |
| | | UCUUUCGUGGUAUAUUACUCUCUG | |
| | | AACUACUACCUCA (SEQ ID NO 5) | |
| 6 | S2_Tlet7f_Cban | GGGAAACUAUACAAUGUAAUAUAA | |
| | 3pR | 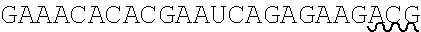 | |
| | | 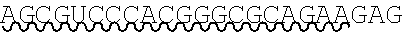 | |
| | | AAGUCAACCAGAGAAACAUAAUAUA | |
| | | CCUACUACCUCA (SEQ ID NO 6) | |

i) General Schematic of the Ribozyme Comprising Two Catalytic Domains and Two Inhibitory Domains (“Toehold”) (e.g. Those Resembled by Structure V in the Detailed Description)

The sequence of S1 contains a cleavage product. In this example, the cleavage product has the sequence {GUCCUUAGUCGAAAGUUUUACUAGAGUCA} (SEQ ID NO 25, in which the underlined nucleotides correspond to the reverse sequence of dme-ban3p (the variable “releasable RNA segment” as defined in the detailed description). Note that the GUCC on the 5′ end and the CA on the 3′ end are essential for the cleavage at the cleavage sites and are thus not part of the “releasable RNA segment”, but are part of the cleavage product. A schematised version of S1 and S2, where (n)ₐ and (n)_{b} represent sequences complementary to the target miRNA, and the cleavage product is demarcated by { }, is as follows:

S1 sequence:
(SEQ ID NO.: 8)
5′-(n)ₐGUAUAUUAGUAUAUUACCUGGUUUUCGAUCGAAA
GAUCGACGAGGUGAAAACCUCGUGACA{GUCCUUAGUCGA
AAGUUUUACUAGAGUCA}GUCCUGAUUGUGCUCGAAAG
AGCACAGACCUGAAAAGGUCUUUCGUGGUAUAUUACUCUCUG
AA(n)_{b}-3′
S2 sequence:.
(SEQ ID NO.: 9)
5′-{n)ₐGUAAUAUAAGAAACACACGAAUCAGAGAAGACGA
GCGUCCCACGGGCGCAGAAGAGAAGUCAACCAGAGAAACAU
AAUAUAC(n)_{b}-3′
ii) Example ribozvme sequences:
Core S1 template (can be used as template
for PCR of S1 for all ribozymes)
(SEQ ID NO.: 10):
5′-CTGGTTTTCGATCGAAAGATCGACGAGGTGAAAACCTC
GTGACAGTCCTTAGTCGAAAGTTTTACTAGAGTCAGTCCTG
ATTGTGCTCGAAAGAGCAC-3′
Core S2 template (can be used as template
for PCR of S1 for all ribozymes)
(SEQ ID NO.: 11):
5′-AGAAACACACGAATCAGAGAAGACGAGCGTCCCACGG
GCGCAGAAGAGAAGTCA-3′

### Example 1: Drosophila Bantam-5p Ribozyme

Sequence of dme-ban-5p:
(SEQ ID NO.: 2)
5′-CCGGUUUUCGAUUUGGUUUGACU-3′
Reverse complement of 5′ paired sequence:
(SEQ ID NO.: 12)
AUCGAAAACCGG.
Reverse complement of 3′ paired sequence:
(SEQ ID NO.: 13)
AGUCAAACCAA.
S1 forward primer (for dme-ban-5p):
(T7 promoter sequence is underlined)
(SEQ ID NO.: 14):
5′-GTATAATACGACTCACTATAGGGAAGTCAAACCAAGTA
TATTAGTATATTACCTGGTTTTCGATCGAAAGATC-3′
S1 reverse primer (for dme-ban-5p)
(SEQ ID NO.: 15):
5′-CCGGTTTTCGATTTCAGAGAGTAATATACCACGAAAGA
CCTTTTCAGGTCTGTGCTCTTTCGAGCACAATC-3′
S2 forward primer (for dme-ban-5p):
(T7 promoter sequence is underlined)
(SEQ ID NO.: 16):
5′-GTATAATACGACTCACTATAGGGAAGTCAAACCAAGTAA
TATAAGAAACACACGAATCAGAGAA-3′

### Example 2: Human Let-7f-5p Ribozyme

Sequence of hsa-let-7f-5p:
(SEQ ID NO.: 4)
5′-UGAGGUAGUAGAUUGUAUAGUU-3′
Reverse complement of 5′ paired sequence:
(SEQ ID NO.: 18)
CUACUACCUCA.
Reverse complement of 3′ paired sequence:
(SEQ ID NO.: 19)
AACUAUACAAU.
S1 forward primer (for hsa-let-7f-5p):
(17 promoter sequence is underlined)
(SEQ ID NO.: 20):
5′-GTATAATACGACTCACTATAGGGAAACTATACAATG
TATATTAGTATATTACCTGGTTTTCGATCGAAAGATC-3′
S1 reverse primer (for hsa-let-7f-5p)
(SEQ ID NO.: 21):
5′-TGAGGTAGTAGTTCAGAGAGTAATATACCACGAAAG
ACCTTTTCAGGTCTGTGCTCTT TCGAGCACAATC-3′
S2 forward primer (for hsa-let-7f-5p):
(17 promoter sequence is underlined)
(SEQ ID NO.: 22):
5′-GTATAATACGACTCACTATAGGGAAACTATACAATG
TAATATAAGAAACACACGAATCAGAGAA-3′
S2 reverse primer (for hsa-let-7f-5p)
(SEQ ID NO.: 23):
5′-TGAGGTAGTAGGTATATTATGTTTCTCTGGTTGACT
TCTCTTCTGCG-3′
Resulting S1 strand for hsa-let-7f-5p
ribozyme
(SEQ ID NO.: 5):
5′-GGGAAACUAUACAAUGUAUAUUAGUAUAUUACCUG
GUUUUCGAUCGAAAGAUCGACGAGGUGAAAACCUCGUG
ACAGUCCUUAGUCGAAAGUUUUACUAGAGUCAGUCCUG
AUUGUGCUCGAAAGAGCACAGACCUGAAAAGGUCUUUC
GUGGUAUAUUACUCUCUGA ACUACUACCUCA-3′
Resulting S2 strand for hsa-let-7f-5p
ribozyme
(SEQ ID NO.: 6):
5′-GGGAAACUAUACAAUGUAAUAUAAGAAACACACGA
AUCAGAGAAGACGAGCGUCCCACGGGCGCAGAAGAGAA
GUCAACCAGAGAAACAUAAUAUACCUACUACCUCA-3′

B. Examples of Ribozyme Targeting Variously Sized Viral RNA Sequences from SARS-CoV-2 Viral Genome (Orf1ab and E Gene), Cleaving Out Dme-ban3p-Reverse Sequence)

Two genes were selected for detection based on usage in other protocols for SARS-CoV-2 detection: Orf1ab gene and E gene. Note that sequences used here differ slightly from the exact sequences used in other methods referred to above. As target sequences were long, target RNA were made via in vitro transcription, and the resultant RNA contained a 5′ T7 promoter sequence (5′-GTATAATACGACTCACTATAGGGA-3′) (SEQ ID NO.: 7). Sequences of ribozymes and target sequences are summarized in the table below (T7 promoter sequence underlined).

TABLE 2
Examples of ribozvme targeting variously
sized viral RNA sequences from SARS-CoV-2
viral genome (Orflab and E gene),
cleaving out dme-ban3p-reverse sequence)

| | | | Name of |
|---|---|---|---|
| | | | ribozyme |
| SEQ | Name of RNA | Sequence of | formed by |
| ID | or | RNA | S1 and |
| NO. | sequence | (5′ to 3′) | S2 |
| 40 | Target RNA | GUAUAAUACGACUCA | TOrf1ab20_ |
| | T7_Orf1ab_20 | CUAUAGGGACAGAUC | Cban3PR |
| | | UAAUGGCUGC | |
| 41 | S1_Orf1ab _20 | GCAGCCAUUAGUAUA | |
| | | UUAGUAUAUUACCUG | |
| | | GUUUUCGAUCGAAAG | |
| | | AUCGACGAGGUGAAA | |
| | | ACCUCGUGACAGUCC | |
| | | UUAGUCGAAAGUUUU | |
| | | ACUAGAGUCAGUCCU | |
| | | GAUUGUGCUCGAAAG | |
| | | AGCACAGACCUGAAA | |
| | | AGGUCUUUCGUGGUA | |
| | | UAUUACUCUCUGAAG | |
| | | AUCUGUCCC | |
| 42 | S2_Orf1ab_20 | GCAGCCAUUAGUAAU | |
| | | AUAAGAAACACACGA | |
| | | AUCAGAGAAGACGAG | |
| | | CGUCCCACGGGCGCA | |
| | | GAAGAGAAGUCAACC | |
| | | AGAGAAACAUAAUAU | |
| | | ACGAUCUGUCCC | |
| 43 | Target RNA | GUAUAAUACGACUCA | TOrflab40_ |
| | T7_Orf1ab_40 | CUAUAGGGAGUAGAC | Cban3pR |
| | | AAUUCUAGUCUUACU | |
| | | AUUAAGAAACCUAAU | |
| 44 | S1_Orf1ab _40 | AUUAGGUUUCUUAAU | |
| | | AGUAAGUAUAUUAGU | |
| | | AUAUUACCUGGUUUU | |
| | | CGAUCGAAAGAUCGA | |
| | | CGAGGUGAAAACCUC | |
| | | GUGACAGUCCUUAGU | |
| | | CGAAAGUUUUACUAG | |
| | | AGUCAGUCCUGAUUG | |
| | | UGCUCGAAAGAGCAC | |
| | | AGACCUGAAAAGGUC | |
| | | UUUCGUGGUAUAUUA | |
| | | CUCUCUGAAGACUAG | |
| | | AAUUGUCUACUCCC | |
| 45 | S2_Orf1ab_40 | AUUAGGUUUCUUAAU | |
| | | AGUAAGUAAUAUAAG | |
| | | AAACACACGAAUCAG | |
| | | AGAAGACGAGCGUCC | |
| | | CACGGGCGCAGAAGA | |
| | | GAAGUCAACCAGAGA | |
| | | AACAUAAUAUACGAC | |
| | | UAGAAUUGUCUACUC | |
| | | CC | |
| 46 | T7_Egene_60 | GUAUAAUACGACUCA | TEgene_ |
| | | CUAUAGGGACUGCGC | Cban3pR |
| | | UUCGAUUGUGUGCGU | |
| | | ACUGCUGCAAUAUUG | |
| | | UUAACGUGAGUCUUG | |
| | | UAAAA | |
| 47 | S1_E_gene_60 | UUUUACAAGACUCAC | |
| | | GUUAACAAUAUUGCA | |
| | | GUAUAUUAGUAUAUU | |
| | | ACCUGGUUUUCGAUC | |
| | | GAAAGAUCGACGAGG | |
| | | UGAAAACCUCGUGAC | |
| | | AGUCCUUAGUCGAAA | |
| | | GUUUUACUAGAGUCA | |
| | | GUCCUGAUUGUGCUC | |
| | | GAAAGAGCACAGACC | |
| | | UGAAAAGGUCUUUCG | |
| | | UGGUAUAUUACUCUC | |
| | | UGAAGCAGUACGCAC | |
| | | ACAAUCGAAGCGCAG | |
| | | UCCC | |
| 48 | S2_Egene_60 | UUUUACAAGACUCAC | |
| | | GUUAACAAUAUUGCA | |
| | | GUAAUAUAAGAAACA | |
| | | CACGAAUCAGAGAAG | |
| | | ACGAGCGUCCCACGG | |
| | | GCGCAGAAGAGAAGU | |
| | | CAACCAGAGAAACAU | |
| | | AAUAUACGCAGUACG | |
| | | CACACAAUCGAAGCG | |
| | | CAGUCCC | |

Primers used to amplify S1 and S2 strands of respective ribozymes:

S1_Orf1ab_gene_20_F (SEQ ID NO. 52):
5′-GTATAATACGACTCACTATAGGGAGCAGCCATTAGTAT
ATTAGTATATTACCTGGTTTTCGATCGAAAGATC-3′
S1_Orf1ab_gene_20_R (SEQ ID NO. 53):
5′-GGGACAGATCTTCAGAGAGTAATATACCACGAAAGACC
TTTTCAGGTCTGTGCTCTTTCGAGCACAATC-3′
S2_Orf1ab_gene_20_F (SEQ ID NO. 54):
5′-GTATAATACGACTCACTATAGGGAGCAGCCATTAGTAA
TATAAGAAACACACGAATCAGAGAA-3′
52_Orf1ab_gene_20_R (SEQ ID NO. 55):
5′-GGGACAGATCGTATATTATGTTTCTCTGGTTGACTTCT
CTTCTGCG-3′
S1_Orf1ab_gene_40_F (SEQ ID NO. 56):
5′-GTATAATACGACTCACTATAGGGAATTAGGTTTCTTAA
TAGTAAGTATATTAGTATATTACCTGGTTTTCGATCGAAAG
ATC-3′
S1_Orf1ab_gene_40_R (SEQ ID NO. 57):
5′-GGGAGTAGACAATTCTAGTCTTCAGAGAGTAATATACC
ACGAAAGACCTTTTCAGGTCTGTGCTCTTTCGAGCACAAT
C-3′
S2_Orf1ab_gene_40_F (SEQ ID NO. 58):
5′-GTATAATACGACTCACTATAGGGAATTAGGTTTCTTAA
TAGTAAGTAATATAAGAAACACACGAATCAGAGAA-3′
S2_Orf1ab_gene_40_R (SEQ ID NO. 59):
5′-GGGAGTAGACAATTCTAGTCGTATATTATGTTTCTCTG
GTTGACTTCTCTTCTGCG-3′
S1_E_gene_60_F (SEQ ID NO. 60):
5′-GTATAATACGACTCACTATAGGGATTTTACAAGACTCA
CGTTAACAATATTGCAGTATATTAGTATATTACCTGGTTTT
CGATCGAAAGATC-3′
5′-GGGACTGCGCTTCGATTGTGTGCGTACTGCTTCAGAGA
GTAATATACCACGAAAGACCTTTTCAGGTCTGTGCTCTTTC
GAGCACAATC-3′
5′-GTATAATACGACTCACTATAGGGATTTTACAAGACTCA
CGTTAACAATATTGCAGTAATATAAGAAACACACGAATCAG
AGAA-3′
S2_E_gene_60_R (SEQ ID NO. 63):
5′-GGGACTGCGCTTCGATTGTGTGCGTACTGCGTATATTA
TGTTTCTCTGGTTGACTTCTCTTCTGCG-3′.

The ribozymes mentioned above can also be arranged in “duplicated tandem ribozyme) configuration, as illustrated in FIGS. 1G to 1I. Table 3 provides example sequences of ribozymes in such configurations (specifically the configuration illustrated in FIG. 1I) targeting the microRNAs dme-ban-5p, hsa-let-7f-5p, and various lengths of viral Orf1ab gene fragments.

TABLE 3
Example sequences of ribozymes in such configurations (specifically the
configuration illustrated in FIG. 1F). The underlined ( ) sequences represent

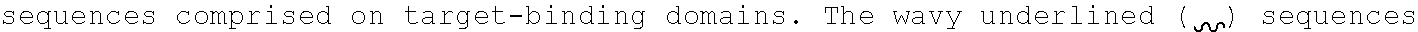

represent the sequences comprised on the releasable RNA segment, or the sequences
comprised on the region opposing and at least partially complementary to
the releasable RNA segment.

| SEQ ID | | |
|---|---|---|
| NO. | Sequence of RNA (5′ to 3′) | Name of RNA or sequence |
| 92 | GGGAAGUCAAACCAAGUAUAUUAGUAUAUU | S1_Tban5p_Cban3pR_Dup |
| | ACCUGGUUUUCGAUCGAAAGAUCGACGAG | |
| | 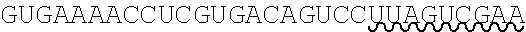 | |
| | 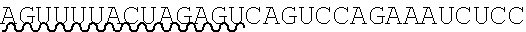 | |
| | CUCACUUCGGUGAGGGAGAUUUCUGAGAA | |
| | GACACCAGAGAAACAUAAUAUAC | |
| | AUCGAAAACCGG | |
| 93 | GGGAAGUCAAACCAAGUAUAUUAGUAUAUU | S2_Tban5p_Cban3pR_Dup |
| | ACCUGGUGUCCCACGGGCGCAGAAGAGAA | |
| | GUCAACCAGAGAAACAUAAUAUACAUCGAA | |
| | AACCGG | |
| 94 | GGGAAACUAUACAAUGUAUAUUAGUAUAUU | S1_Tlet7f_Cban3pR_Dup |
| | ACCUGGUUUUCGAUCGAAAGAUCGACGAG | |
| | 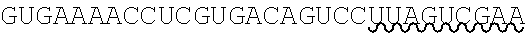 | |
| | 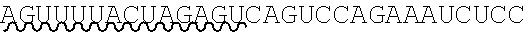 | |
| | CUCACUUCGGUGAGGGAGAUUUCUGAGAA | |
| | GACACCAGAGAAACAUAAUAUACCUACUAC | |
| | CUCA | |
| 95 | GGGAAACUAUACAAUGUAUAUUAGUAUAUU | S2_Tlet7f_Cban3pR_Dup |
| | ACCUGGUGUCCCACGGGCGCAGAAGAGAA | |
| | GUCAACCAGAGAAACAUAAUAUACCUACUA | |
| | CCUCA | |
| 96 | GGGAGCAGCCAUUAGUAUAUUAGUAUAUUA | S1_T_Orf1ab_20_Cban3pR_ |
| | CCUGGUUUUCGAUCGAAAGAUCGACGAGG | Dup |
| | 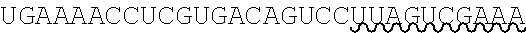 | |
| | 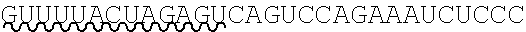 | |
| | UCACUUCGGUGAGGGAGAUUUCUGAGAAG | |
| | ACACCAGAGAAACAUAAUAUACGAUCUGUC | |
| | CC | |
| 97 | GGGAGCAGCCAUUAGUAUAUUAGUAUAUUA | S2_T_Orf1ab_20_Cban3pR_ |
| | CCUGGUGUCCCACGGGCGCAGAAGAGAAG | Dup |
| | UCAACCAGAGAAACAUAAUAUACGAUCUGU | |
| | CCC | |
| 98 | GGGAAUUAGGUUUCUUAAUAGUAAGUAUAU | Si _T _Orf 1 ab 40 Cban3pR_ |
| | UAGUAUAUUACCUGGUUUUCGAUCGAAAGA | Dup |
| | UCGACGAGGUGAAAACCUCGUGACAGUCC | |
| | 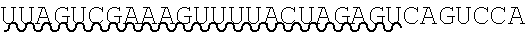 | |
| | GAAAUCUCCCUCACUUCGGUGAGGGAGAU | |
| | UUCUGAGAAGACACCAGAGAAACAUAAUAU | |
| | ACGACUAGAAUUGUCUACUCCC | |
| 99 | GGGAAUUAGGUUUCUUAAUAGUAAGUAUAU | S2_T_Orf1ab_40_Cban3pR_ |
| | UAGUAUAUUACCUGGUGUCCCACGGGCGC | Dup |
| | AGAAGAGAAGUCAACCAGAGAAACAUAAUA | |
| | UACGACUAGAAUUGUCUACUCCC | |
| 100 | GGGAUUUUACAAGACUCACGUUAACAAUAU | S1_T_Egene_60_Cban3pR_ |
| | UGCAGUAUAUUAGUAUAUUACCUGGUUUUC | Dup |
| | GAUCGAAAGAUCGACGAGGUGAAAACCUCG | |
| | 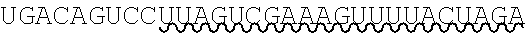 | |
| | 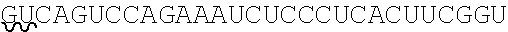 | |
| | GAGGGAGAUUUCUGAGAAGACACCAGAGAA | |
| | ACAUAAUAUACGCAGUACGCACACAAUCGA | |
| | AGCGCAGUCCC | |
| 101 | GGGAUUUUACAAGACUCACGUUAACAAUAU | S2_T_Egene_60_Cban3pR_ |
| | UGCAGUAUAUUAGUAUAUUACCUGGUGUC | Dup |
| | CCACGGGCGCAGAAGAGAAGUCAACCAGA | |
| | GAAACAUAAUAUACGCAGUACGCACACAAU | |
| | CGAAGCGCAGUCCC | |

### Example 3

The ribozymes described above all comprise inhibitory domains (“toehold”) for the minimization of background cleavage activity. As described in the detailed description, we also disclose herein ribozymes without the toehold features (e.g. those resembled by structure Ill in the detailed description). The table below provides sequences of the ribozymes (without toehold) targeting the microRNAs dme-ban-5p, hsa-let-7f-5p, and various lengths of viral Orf1ab and E gene fragments.

The ribozymes in the table below follow the general S1 sequence of:

(SEQ ID.: 80)
5′-(n)ₐGGGAAGUGGUAUAUUACCUGGUUUUCGAUCGAAAGAU
CGACGAGGUGAAAACCUCGUGACAGUCC(n)ₓCAGUCCUGAUUG
UGCUCGAAAGAGCACAGACCUGAAAAGGUCUUUCGUGGUAUAUU
ACCUGGUCCC (n)_{b}-3′,

and the general S2 sequence of:

(SEQ ID.: 83)
5′-(n)ₐGGGACCAGAGAAACACACGAAUCAGAGAAG(n)ₓGAG
AAGUCAACCAGAGAAACACACUUCCC(n)_{b}-3′,

wherein (n)ₓ represents a releasable RNA segment, and (n)ₐ and (n)_{b} represent sequences complementary to the first and second target RNA molecules.

TABLE 4
Examples of ribozymes without toeholds (inhibitory domains). The underlined
( ) sequences represent sequences comprised on target-binding domains. The wavy

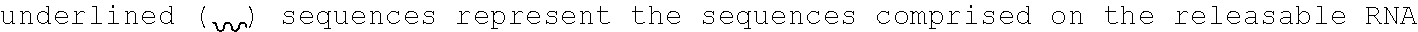

segment, or the sequences comprised on the region opposing and at least partially
complementary to the releasable RNA segment.

| SEQ ID | | |
|---|---|---|
| NO.: | Sequence | Remarks |
| 70 | GGGAAGUCAAACCAAGGGAAGUG | S1 strand of dme-ban- |
| | GUAUAUUACCUGGUUUUCGAUCGAAAGAUC | 5p without toehold |
| | 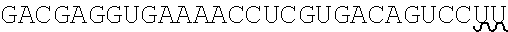 | |
| | 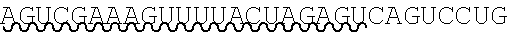 | |
| | AUUGUGCUCGAAAGAGCACAGACCUGAAAA | |
| | GGUCUUUCGUGGUAUAUUACCUGGUCCCA | |
| | UCGAAAACCGG | |
| 71 | GGGAAGUCAAACCAAGGGACCAG | S2 strand of dme-ban- |
| | 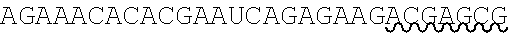 | 5p without toehold |
| | 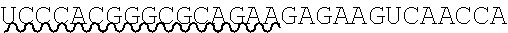 | |
| | GAGAAACACACUUCCCAUCGAAAACCGG | |
| 72 | GGGAAACUAUACAAUGGGAAGUG | S1 strand of hsa-let-7f- |
| | GUAUAUUACCUGGUUUUCGAUCGAAAGAUC | 5p without toehold |
| | 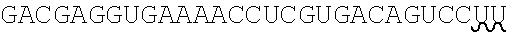 | |
| | 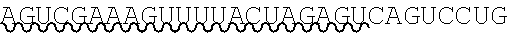 | |
| | AUUGUGCUCGAAAGAGCACAGACCUGAAAA | |
| | GGUCUUUCGUGGUAUAUUAC | |
| | CUGGUCCCCUACUACCUCA | |
| 73 | GGGAAACUAUACAAUGGGACCAG | S2 strand of hsa-let-7f- |
| | 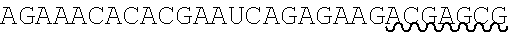 | 5p without toehold |
| | 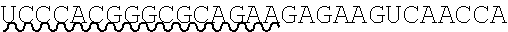 | |
| | GAGAAACACACUUCCCCUACUACCUCA | |
| 74 | GCAGCCAUUAGGGAAGUG | S1 strand of |
| | GUAUAUUACCUGGUUUUCGAUCGAAAGAUC | Orf1ab_gene_20nt |
| | 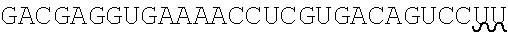 | without toehold |
| | 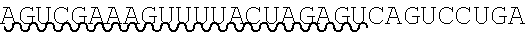 | |
| | UUGUGCUCGAAAGAGCACAGACCUGAAAAG | |
| | GUCUUUCGUGGUAUAUUACCUGGUCCC | |
| | GAUCUGUCCC | |
| 75 | GCAGCCAUUAGGGACCAG | S2 strand of |
| | 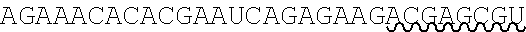 | Orf1ab_gene_20nt |
| | 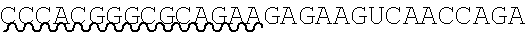 | without toehold |
| | GAAACACACUUCCCGAUCUGUCCC | |
| 76 | AUUAGGUUUCUUAAUAGUAAGGGAAGUG | S1strand of |
| | GUAUAUUACCUGGUUUUCGAUCGAAAGAUC | Orf1ab_gene_40nt |
| | 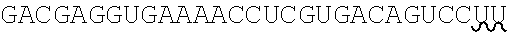 | without toehold |
| | 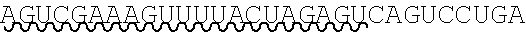 | |
| | UUGUGCUCGAAAGAGCACAGACCUGAAAAG | |
| | GUCUUUCGUGGUAUAUUACCUGGUCCC | |
| | GACUAGAAUUGUCUACUCCC | |
| 77 | AUUAGGUUUCUUAAUAGUAAGGGACCAG | S2 strand of |
| | 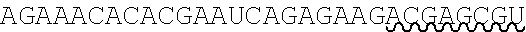 | Orf1ab_gene_40nt |
| | 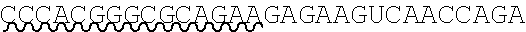 | without toehold |
| | GAAACACACUUCCC | |
| | GACUAGAAUUGUCUACUCCC | |
| 78 | UUUUACAAGACUCACGUUAACAAUAUUGCA | S1 strand of |
| | GGGAAGUG | E_gene_60nt without |
| | GUAUAUUACCUGGUUUUCGAUCGAAAGAUC | toehold |
| | 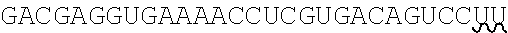 | |
| | 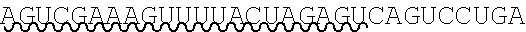 | |
| | UUGUGCUCGAAAGAGCACAGACCUGAAAAG | |
| | GUCUUUCGUGGUAUAUUACCUGGUCCC | |
| | GCAGUACGCACACAAUCGAAGCGCAGUCCC | |
| 79 | UUUUACAAGACUCACGUUAACAAUAUUGCA | S2 strand of |
| | GGGACCAG | E_gene_60t without |
| | 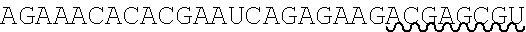 | toehold |
| | 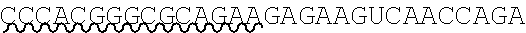 | |
| | GAAACACACUUCCC | |
| | GCAGUACGCACACAAUCGAAGCGCAGUCCC | |

### Example 4

The ribozymes can be in the single-catalytic domain configuration. These ribozymes comprises only 1 RNA strand, as resembled by structures I and II in the detailed description.

A general sequence for the single-catalytic domain ribozymes is provided below:

(SEQ ID.: 88)
5′-(n)ₐGUAUAUUAGUAUAUUACCUGGUUUUCGAUCGAAAGAU
CGACGAGGUGAAAACCUCGUGACAGUCC(n)ₓCAGUCCUGAUUG
UGAUCGAAAGAGCACAGACCUGAAAAGGUCUUUCUGGUUUCGAC
CAGAAUCAGAGAAG(n)_{y}GAGAAGUCAACCAGAGAAACAUAAUA
UAC(n)_{b}-3′,

wherein (n)ₐ and (n)_{b} represent sequences complementary to the first and second target RNA molecules; (n)ₓ represents a releasable RNA segment, and (n)_{y} represents a sequence complementary to the releasable RNA segment.

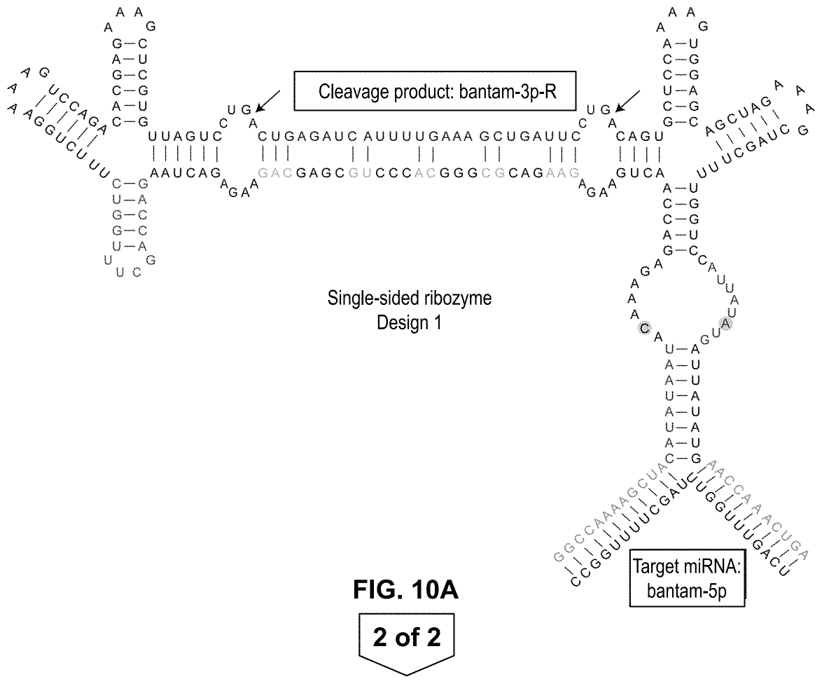

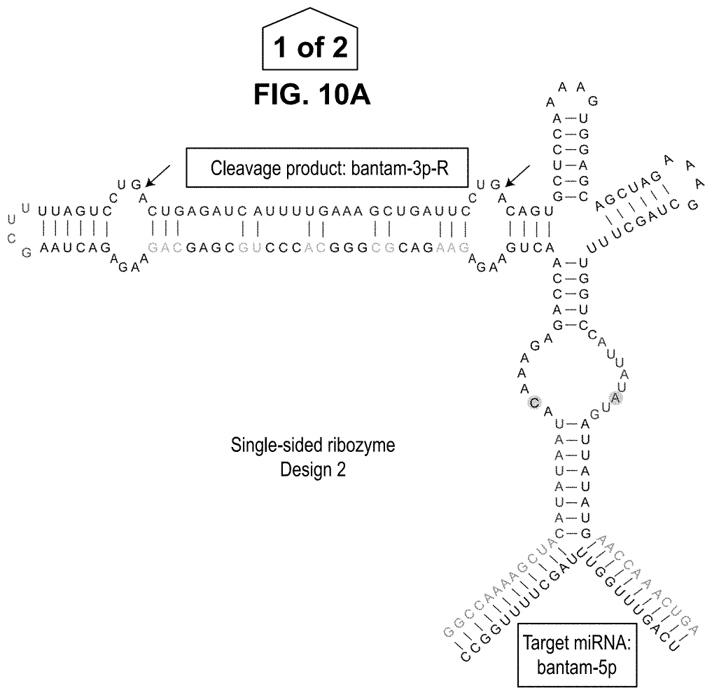

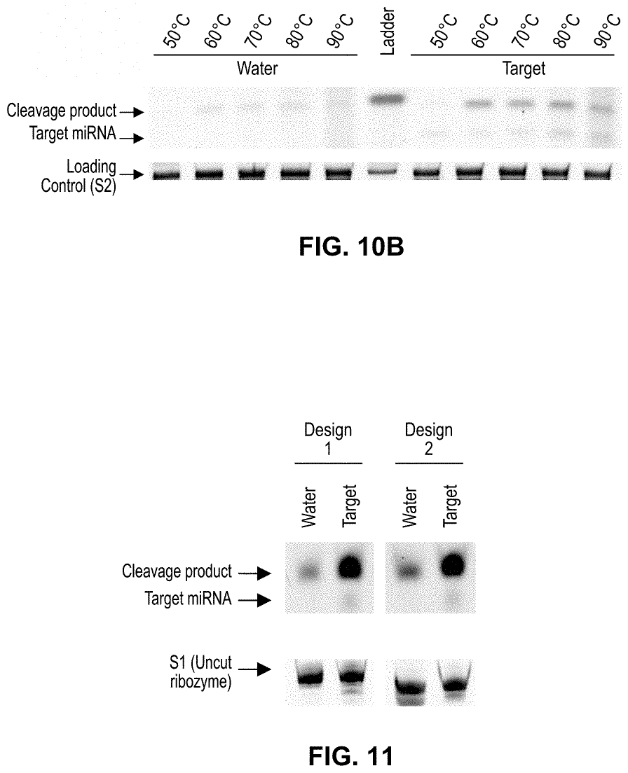

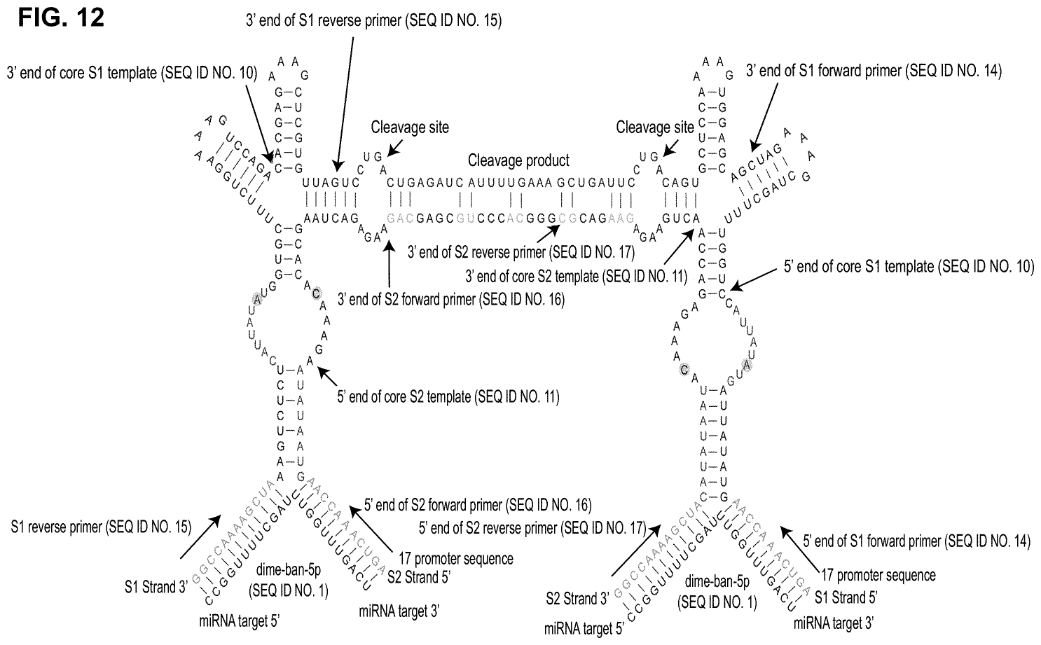

The sequences for the 2 specific ribozymes tested in FIGS. 10A and 10B are provided in table 5:

TABLE 5
Examples of ribozymes comprising a single-catalytic domain. The single underlined
( ) sequences represent sequences comprised on target-binding domains. The wavy

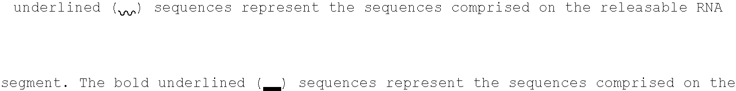

region opposing and at least partially complementary to the releasable RNA segment.

| | | |
|---|---|---|
| 86 | GGGAAGUCAAACCAAGUAUAUUAGUAUAU | Tban5p_Cban3pR_design1_ |
| | UACCUGGUUUUCGAUCGAAAGAUCGACGA | full_length |
| | 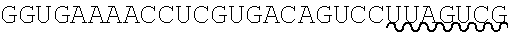 | |
| | 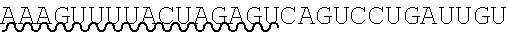 | |
| | GAUCGAAAGAGCACAGACCUGAAAAGGUCU | |
| | 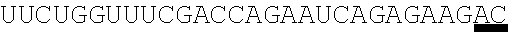 | |
| | 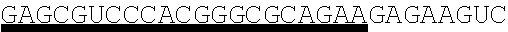 | |
| | AACCAGAGAAACAUAAUAUACAUCGAAAAC | |
| | CGG | |
| 87 | GGGAAGUCAAACCAAGUAUAUUAGUAUAU | Tban5p_Cban3pR_design2_ |
| | UACCUGGUUUUCGAUCGAAAGAUCGACGA | full_length |
| | 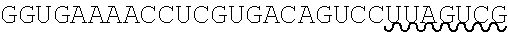 | |
| | 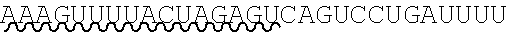 | |
| | 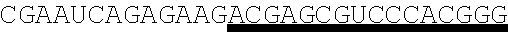 | |
| | 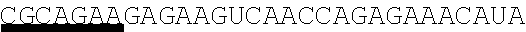 | |
| | AUAUACAUCGAAAACCGG | |

### 3. Results

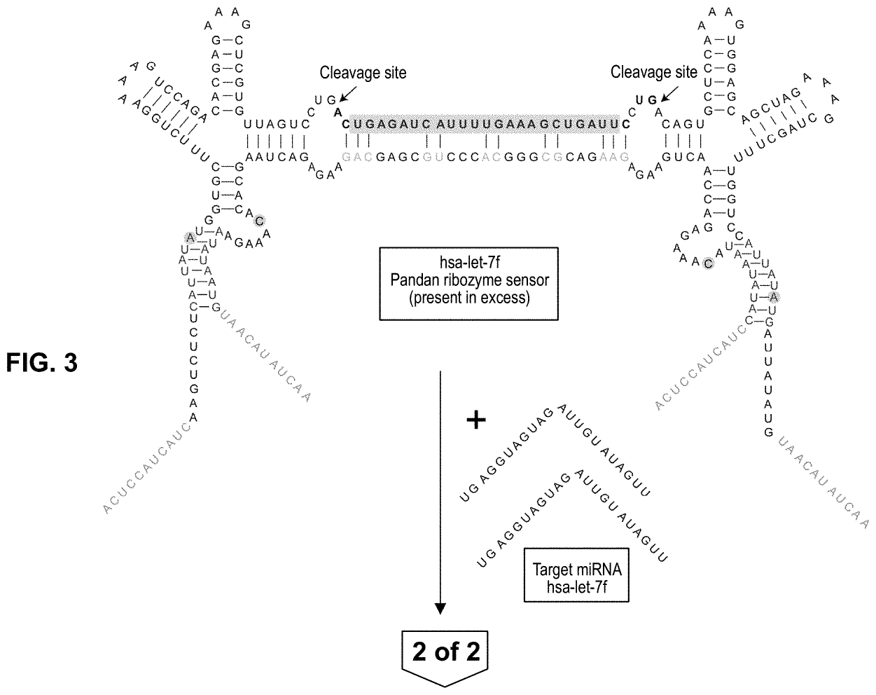

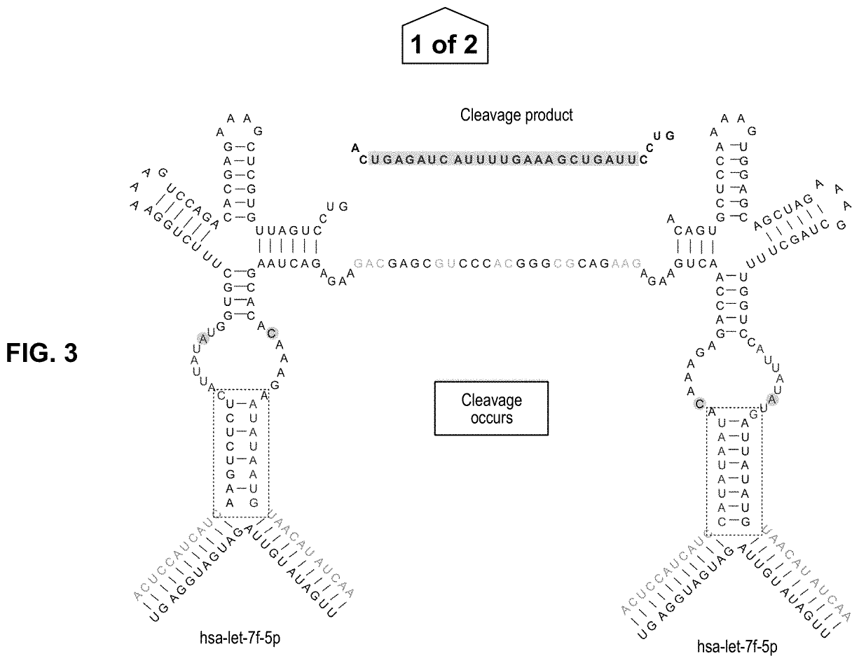

FIG. 3 shows the working principles of an exemplary ribozyme. In this example, the ribozyme is reverse-joined by two hairpin ribozymes, each with a modified Helix 4 with a variable stem region and complementary sequence to the target miRNA, are arranged in tandem. The variable stem connecting the ribozyme to the complementary arms for the target RNA is designed to have partial complementarity to the catalytic loop, acting as a “toehold” to minimize self-cleavage. Without the target RNA, no cleavage occurs, and no cleavage product is released. Target RNA binding during the annealing cycle activates cleavage, releasing an RNA product (the sequence of this product can be varied, and has loose complementarity to the ribozyme so that cleavage is favoured over re-ligation); both the RNA cleavage product and target miRNA are released during the melt cycle. The released target RNAs hence can bind anew to uncleaved ribozymes (present in excess) in the next annealing cycle. As the ribozyme is in excess, a single target miRNA can activate self-cleavage of multiple tandem HpRzs, leading to the release of multiple cleavage products, hence amplifying the target RNA signal.

In addition, the cleavage product sequence can be the same as that of that of the target RNA, hence allowing exponential amplification.)

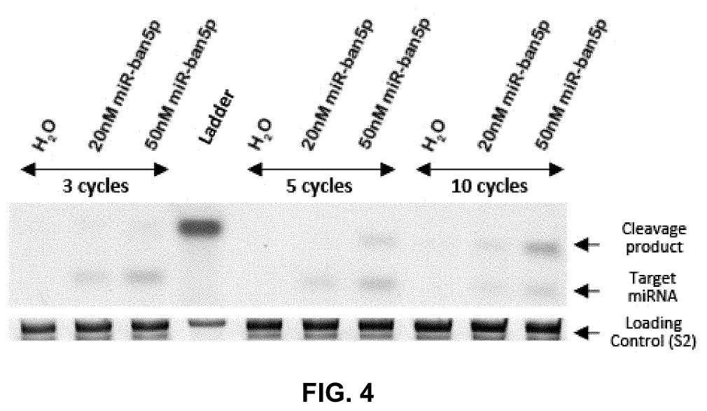

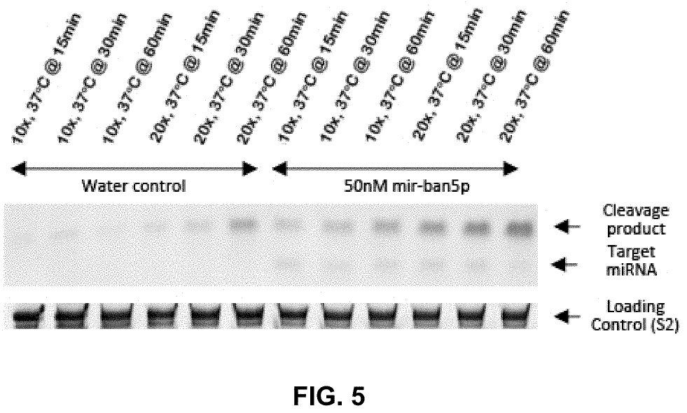

FIGS. 4 and 5 show results of the ribozyme for miRNA dme-bantam-5p tested with its target. Increasing miRNA levels (FIG. 4), and increasing cycle number (FIGS. 4 and 5) led to 3-4-fold increase in the level of cleavage product produced. Since ribozyme was provided at 4× molar ratio over that of target, this demonstrates that the provided ribozyme was completely cleaved.

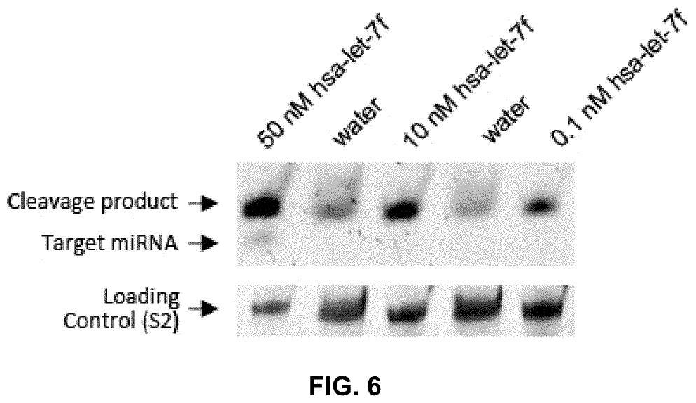

The ribozyme also works for human miRNAs, as demonstrated by the hsa-let-7f sensor (FIG. 6). When the amount of let-7f sensor was provided at 2000-fold molar ratio over that of the target miRNA (200 nM sensor vs 0.1 nM target), the target signal was amplified ˜200 fold (FIG. 6).

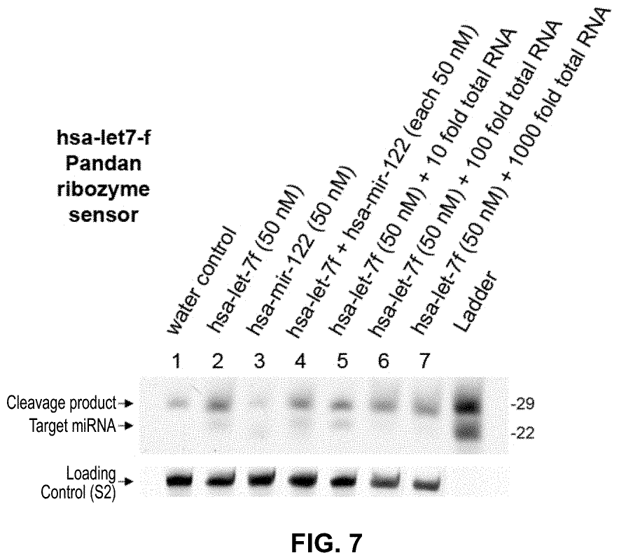

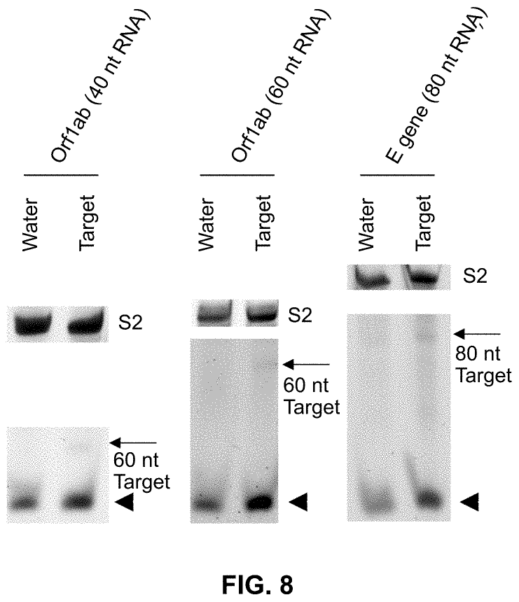

The ribozymes are specific, able to distinguish between its target RNA and a different microRNA, and can detect their target from within a complex mixture of RNAs at 1000-fold greater concentration than the target RNA (FIG. 7).

The ribozyme can detect and amplify target RNA molecules with 40 nt, 60 nt and 80 nt in length, containing RNA sequence fragments of genes (Orf1ab and E gene) from the genome of coronavirus SARS-CoV-2. 200 nM of ribozyme and 50 nM of target RNA are provided in each reaction.

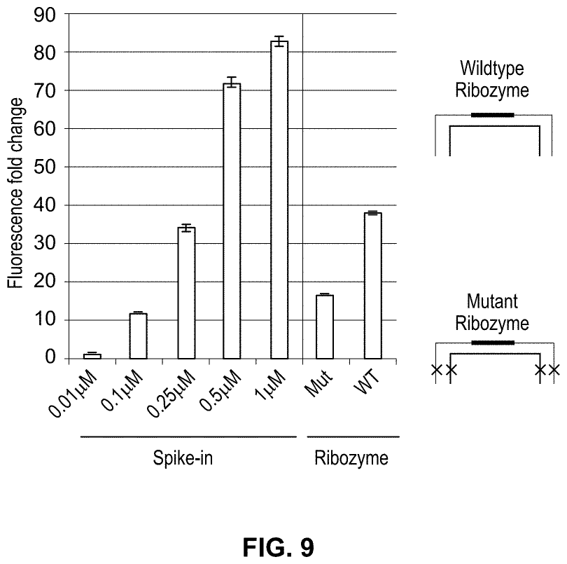

Cleavage products generated by self-cleavage of ribozyme can be used to activate Pandan fluorescence, as compared with mutant ribozyme that is unable to cleave (FIG. 9).

We have also tested ribozymes with the single catalytic domain configuration. The single catalytic domain configuration is capable of cleaving two cleavage sites to cause release of the RNA product. Two “single catalytic domain” configurations have been tested, both targeting the microRNA dme-ban-5p. (B) Both designs 1 and 2 are effective in releasing the cleavage products (ban3p-reverse RNA) upon incubation with the target RNA molecules (dme-ban-5p).

Melting temperatures ranging from 50° C. to 90° C. were tested in the cleavage reactions for ribozymes targeting dme-ban-5p (the target RNA molecule). The temperature ranging from above 50° C. to 90° C. is particularly effective in promoting the release of the cleavage products.

4. Sequence listing table

| SEQ ID | | |
|---|---|---|
| NO. | Sequence | Remarks |
| 1 | CCGGUUUUCGAUUUGGUUUGACU | Sequence of exemplary target |
| | | RNA dme-bantam-5p |
| 2 | GGGAAGUCAAACCAAGUAUAUUAGUAUAUU | Resulting S1 strand for dme- |
| | ACCUGGUUUUCGAUCGAAAGAUCGACGAG | ban-5p-ribozyme |
| | GUGAAAACCUCGUGACAGUCCUUAGUCGAA | (S1_Tban5p_Cban3pR) |
| | AGUUUUACUAGAGUCAGUCCUGAUUGUGC | |
| | UCGAAAGAGCACAGACCUGAAAAGGUCUUU | |
| | CGUGGUAUAUUACUCUCUGAAAUCGAAAAC | |
| | CGG | |
| 3 | GGGAAGUCAAACCAAGUAAUAUAAGAAACA | Resulting S2 strand for dme- |
| | CACGAAUCAGAGAAGACGAGCGUCCCACG | ban-5p-ribozyme |
| | GGCGCAGAAGAGAAGUCAACCAGAGAAACA | (S2_Tban5p_Cban3pR) |
| | UAAUAUACAUCGAAAACCGG | |
| 4 | UGAGGUAGUAGAUUGUAUAGUU | Sequence of exemplary target |
| | | RNA hsa-let-7f-5p |
| 5 | GGGAAACUAUACAAUGUAUAUUAGUAUAUU | Resulting S1 strand for hsa- |
| | ACCUGGUUUUCGAUCGAAAGAUCGACGAG | let-7f-5p-ribozyme |
| | GUGAAAACCUCGUGACAGUCCUUAGUCGAA | (S1_Tlet7f_Cban3pR) |
| | AGUUUUACUAGAGUCAGUCCUGAUUGUGC | |
| | UCGAAAGAGCACAGACCUGAAAAGGUCUUU | |
| | CGUGGUAUAUUACUCUCUGAACUACUACCU | |
| | CA | |
| 6 | GGGAAACUAUACAAUGUAAUAUAAGAAACA | Resulting S2 strand for hsa- |
| | CACGAAUCAGAGAAGACGAGCGUCCCACG | let-7f-5p -ribozyme |
| | GGCGCAGAAGAGAAGUCAACCAGAGAAACA | (S2_Tlet7f_Cban3pR) |
| | UAAUAUACCUACUACCUCA | |
| 7 | GTATAATACGACTCACTATAGGGA | T7 promoter sequence |
| 8 | (n)ₐGUAUAUUAGUAUAUUACCUGGUUUUCG | S1 sequence. The exemplary |
| | AUCGAAAGAUCGACGAGGUGAAAACCUCGU | cleavage product is |
| | GACA{GUCCUUAGUCGAAAGUUUUACUAGA | demarcated by { }, and (n)ₐ |
| | GUCA}GUCCUGAUUGUGCUCGAAAGAGCAC | and (n)_{b} represent sequences |
| | AGACCUGAAAAGGUCUUUCGUGGUAUAUUA | complementary to the target |
| | CUCUCUGAA(n)_{b} | RNA molecule. |
| 9 | (n)ₐGUAAUAUAAGAAACACACGAAUCAGAGA | S2 sequence. (n)ₐ and (n)_{b} |
| | AGACGAGCGUCCCACGGGCGCAGAAGAGA | represent sequences |
| | AGUCAACCAGAGAAACAUAAUAUAC(n)_{b} | complementary to the target |
| | | RNA molecule. |
| 10 | CTGGTTTTCGATCGAAAGATCGACGAGGTG | Core S1 DNA template |
| | AAAACCTCGTGACAGTCCTTAGTCGAAAGTT | |
| | TTACTAGAGTCAGTCCTGATTGTGCTCGAAA | |
| | GAGCAC | |
| 11 | AGAAACACACGAATCAGAGAAGACGAGCGT | Core S2 DNA template |
| | CCCACGGGCGCAGAAGAGAAGTCA | |
| 12 | AUCGAAAACCGG | Reverse complement of 5′ |
| | | paired sequence of dme-ban- |
| | | 5p |
| 13 | AGUCAAACCAA | Reverse complement of 3′ |
| | | paired sequence of dme-ban- |
| | | 5p |
| 14 | GTATAATACGACTCACTATAGGGAAGTCAAA | S1 forward primer for dme- |
| | CCAAGTATATTAGTATATTACCTGGTTTTCGA | ban-5p. T7 promoter |
| | TCGAAAGATC | sequence is underlined. |
| 15 | CCGGTTTTCGATTTCAGAGAGTAATATACCA | S1 reverse primer for dme- |
| | CGAAAGACCTTTTCAGGTCTGTGCTCTTTCG | ban-5p |
| | AGCACAATC | |
| 16 | GTATAATACGACTCACTATAGGGAAGTCAAA | S2 forward primer for dme- |
| | CCAAGTAATATAAGAAACACACGAATCAGAG | ban-5p. T7 promoter |
| | AA | sequence is underlined. |
| 17 | CCGGTTTTCGATGTATATTATGTTTCTCTGGT | S2 reverse primer for dme- |
| | TGACTTCTCTTCTGCG | ban-5p |
| 18 | CUACUACCUCA | Reverse complement of 5′ |
| | | paired sequence of hsa-let-7f- |
| | | 5p |
| 19 | AACUAUACAAU | Reverse complement of 3′ |
| | | paired sequence of hsa-let-7f- |
| | | 5p |
| 20 | GTATAATACGACTCACTATAGGGAAACTATA | S1 forward primer for hsa-let- |
| | CAATGTATATTAGTATATTACCTGGTTTTCGA | 7f-5p. T7 promoter sequence |
| | TCGAAAGATC | is underlined. |
| 21 | TGAGGTAGTAGTTCAGAGAGTAATATACCAC | S1 reverse primer for hsa-let- |
| | GAAAGACCTTTTCAGGTCTGTGCTCTTTCGA | 7f-5p |
| | GCACAATC | |
| 22 | GTATAATACGACTCACTATAGGGAAACTATA | S2 forward primer for hsa-let- |
| | CAATGTAATATAAGAAACACACGAATCAGAG | 7f-5p. T7 promoter sequence |
| | AA | is underlined. |
| 23 | TGAGGTAGTAGGTATATTATGTTTCTCTGGT | S2 reverse primer for hsa-let- |
| | TGACTTCTCTTCTGCG | 7f-5p |
| 24 | UGGAGUGUGACAAUGGUGUUUG | hsa-miR-122-5p, a control |
| | | RNA molecule with a |
| | | dissimilar sequence to a |
| | | target RNA molecule to test |
| | | sequence specificity of the |
| | | ribozymes |
| 25 | GUCCUUAGUCGAAAGUUUUACUAGAGUCA | Exemplary cleavage product |
| | | of SEQ ID NO. 8. |
| 26 | (n)ₐGUAUAUUAGUAUAUUACCUGGUUUUCG | An exemplary S1 sequence |
| | AUCGAAAGAUCGACGAGGUGAAAACCUCGU | with toeholds. (n)ₓ represents |
| | GACAGUCC(n)ₓCAGUCCUGAUUGUGCUCGA | a releasable RNA segment, |
| | AAGAGCACAGACCUGAAAAGGUCUUUCGUG | and (n)ₐ and (n)_{b} represent |
| | GUAUAUUACUCUCUGAA(n)_{b} | sequences complementary to |
| | | the first and second target |
| | | RNA molecules. |
| 27 | GUAUAUUAGUAUAUUACCUGGUUUUCGAUC | Portion of SEQ ID NO.: 26 |
| | GAAAGAUCGACGAGGUGAAAACCUCGUGAC | between (n)ₐ (the first |
| | AGUCC | sequence complementary to |
| | | the target RNA molecule and |
| | | (n)ₓ (the releasable RNA |
| | | segment). |
| 28 | CAGUCCUGAUUGUGCUCGAAAGAGCACAG | Portion of SEQ ID NO.: 26 |
| | ACCUGAAAAGGUCUUUCGUGGUAUAUUACU | between (n)ₓ (the cleavage |
| | CUCUGAA | product) and (n)_{b} (the second |
| | | sequence complementary to |
| | | the target RNA molecule). |
| 29 | (n)ₐGUAAUAUAAGAAACACACGAAUCAGAGA | An exemplary S2 sequence |
| | AG(n)ₓGAGAAGUCAACCAGAGAAACAUAAUA | (with toeholds). (n)ₓ |
| | UAC(n)_{b} | represents a region partially |
| | | complementary to the |
| | | releasable RNA segment on |
| | | S1, and (n)ₐ and (n)_{b} |
| | | represent sequences |
| | | complementary to the second |
| | | and first target RNA |
| | | molecules. |
| 30 | GUAAUAUAAGAAACACACGAAUCAGAGAAG | Portion of SEQ ID NO.: 29 |
| | | between (n)ₐ (the sequence |
| | | complementary to the second |
| | | target RNA molecule and (n)ₓ |
| | | (the region partially |
| | | complementary to the |
| | | releasable RNA segment). |
| 31 | GAGAAGUCAACCAGAGAAACAUAAUAUAC | Portion of SEQ ID NO.: 29 |
| | | between (n)ₓ (the region |
| | | partially complementary to the |
| | | releasable RNA segment) and |
| | | (n)_{b} (the sequence |
| | | complementary to the second |
| | | target RNA molecule). |
| 32 | GUAUAUUAGUAUAUUACCUGGUUUUCGAUC | An exemplary sequence of |
| | GAAAGAUCGACGAGGUGAAAACCUCGUGAC | the region of S1 between |
| | AGUCCUUAGUCGAAAGUUUUACUAGAGUCA | motifs [A] and [A′], in the |
| | GUCCUGAUUGUGCUCGAAAGAGCACAGAC | direction of [A] to [A′]. |
| | CUGAAAAGGUCUUUCGUGGUAUAUUACUC | |
| | UCUGAA | |
| 33 | GUAAUAUAAGAAACACACGAAUCAGAGAAG | An exemplary sequence of |
| | ACGAGCGUCCCACGGGCGCAGAAGAGAAG | the region of S2 between |
| | UCAACCAGAGAAACAUAAUAUAC | motifs [a′] and [a], in the |
| | | direction of [a′] to [a]. |
| 34 | GUAUAUUACCUGGUUUUCGAUCGAAAGAUC | An exemplary sequence of |
| | GACGAGGUGAAAACCUCGUGACAGUCCUU | the region of S1 between |
| | AGUCGAAAGUUUUACUAGAGUCAGUCCUGA | motifs [B] and [B′], in the |
| | UUGUGCUCGAAAGAGCAC | direction of [B] to [B′]. |
| 35 | AGAAACACACGAAUCAGAGAAGACGAGCGU | An exemplary sequence of |
| | CCCACGGGCGCAGAAGAGAAGUCAACCAGA | the region of S2 between |
| | GAAACA | motifs [b′] and [b], in the |
| | | direction of [b′] to [b]. |
| 36 | AGTCAAACCAAGTATATTAGTATATTACCTG | S1 forward primer for dme- |
| | GTTTTCGATCGAAAGATC | ban-5p without the T7 |
| | | promoter sequence |
| 37 | AGTCAAACCAAGTAATATAAGAAACACACGA | S2 forward primer for dme- |
| | ATCAGAGAA | ban-5p without T7 promoter |
| | | sequence |
| 38 | AACTATACAATGTATATTAGTATATTACCTGG | S1 forward primer for hsa-let- |
| | TTTTCGATCGAAAGATC | 7f-5p without T7 promoter |
| | | sequence |
| 39 | AACTATACAATGTAATATAAGAAACACACGA | S2 forward primer for hsa-let- |
| | ATCAGAGAA | 7f-5p without T7 promoter |
| | | sequence |
| 40 | GUAUAAUACGACUCACUAUAGGGACAGAUC | Target RNA T7_Orf1ab_20 |
| | UAAUGGCUGC | |
| 41 | GCAGCCAUUAGUAUAUUAGUAUAUUACCUG | S1_Orf1ab_20 |
| | GUUUUCGAUCGAAAGAUCGACGAGGUGAA | |
| | AACCUCGUGACAGUCCUUAGUCGAAAGUUU | |
| | UACUAGAGUCAGUCCUGAUUGUGCUCGAA | |
| | AGAGCACAGACCUGAAAAGGUCUUUCGUG | |
| | GUAUAUUACUCUCUGAAGAUCUGUCCC | |
| 42 | GCAGCCAUUAGUAAUAUAAGAAACACACGA | S2_Orf1ab_20 |
| | AUCAGAGAAGACGAGCGUCCCACGGGCGC | |
| | AGAAGAGAAGUCAACCAGAGAAACAUAAUA | |
| | UACGAUCUGUCCC | |
| 43 | GUAUAAUACGACUCACUAUAGGGAGUAGAC | Target RNA T7_Orf1ab_40 |
| | AAUUCUAGUCUUACUAUUAAGAAACCUAAU | |
| 44 | AUUAGGUUUCUUAAUAGUAAGUAUAUUAGU | S1_Orf1ab_40 |
| | AUAUUACCUGGUUUUCGAUCGAAAGAUCGA | |
| | CGAGGUGAAAACCUCGUGACAGUCCUUAG | |
| | UCGAAAGUUUUACUAGAGUCAGUCCUGAUU | |
| | GUGCUCGAAAGAGCACAGACCUGAAAAGGU | |
| | CUUUCGUGGUAUAUUACUCUCUGAAGACUA | |
| | GAAUUGUCUACUCCC | |
| 45 | AUUAGGUUUCUUAAUAGUAAGUAAUAUAAG | S2_Orf1ab_40 |
| | AAACACACGAAUCAGAGAAGACGAGCGUCC | |
| | CACGGGCGCAGAAGAGAAGUCAACCAGAGA | |
| | AACAUAAUAUACGACUAGAAUUGUCUACUC | |
| | CC | |
| 46 | GUAUAAUACGACUCACUAUAGGGACUGCGC | T7_Egene_60 |
| | UUCGAUUGUGUGCGUACUGCUGCAAUAUU | |
| | GUUAACGUGAGUCUUGUAAAA | |
| 47 | UUUUACAAGACUCACGUUAACAAUAUUGCA | S1_E_gene_60 |
| | GUAUAUUAGUAUAUUACCUGGUUUUCGAUC | |
| | GAAAGAUCGACGAGGUGAAAACCUCGUGAC | |
| | AGUCCUUAGUCGAAAGUUUUACUAGAGUCA | |
| | GUCCUGAUUGUGCUCGAAAGAGCACAGAC | |
| | CUGAAAAGGUCUUUCGUGGUAUAUUACUC | |
| | UCUGAAGCAGUACGCACACAAUCGAAGCGC | |
| | AGUCCC | |
| 48 | UUUUACAAGACUCACGUUAACAAUAUUGCA | S2_Egene_60 |
| | GUAAUAUAAGAAACACACGAAUCAGAGAAG | |
| | ACGAGCGUCCCACGGGCGCAGAAGAGAAG | |
| | UCAACCAGAGAAACAUAAUAUACGCAGUAC | |
| | GCACACAAUCGAAGCGCAGUCCC | |
| 49 | CAGAUCUAAUGGCUGC | Target RNA T7_Orf1ab_20 |
| | | without T7 promoter |
| | | sequence |
| 50 | GUAGACAAUUCUAGUCUUACUAUUAAGAAA | Target RNA T7_Orf1ab_40 |
| | CCUAAU | without T7 promoter |
| | | sequence |
| 51 | CUGCGCUUCGAUUGUGUGCGUACUGCUGC | T7_Egene_60 without T7 |
| | AAUAUUGUUAACGUGAGUCUUGUAAAA | promoter sequence |
| 52 | GTATAATACGACTCACTATAGGGAGCAGCCA | S1 forward primer for |
| | TTAGTATATTAGTATATTACCTGGTTTTCGAT | S1_Orf1ab_gene_20_F |
| | CGAAAGATC | |
| 53 | GGGACAGATCTTCAGAGAGTAATATACCACG | S1 reverse primer |
| | AAAGACCTTTTCAGGTCTGTGCTCTTTCGAG | S1_Orf1ab_gene_20_R |
| | CACAATC | |
| 54 | GTATAATACGACTCACTATAGGGAGCAGCCA | S2 forward primer |
| | TTAGTAATATAAGAAACACACGAATCAGAGA | S2_Orf1ab_gene_20_F |
| | A | |
| 55 | GGGACAGATCGTATATTATGTTTCTCTGGTT | S2 reverse primer |
| | GACTTCTCTTCTGCG | S2_Orf1ab_gene_20_R |
| 56 | GTATAATACGACTCACTATAGGGAATTAGGT | S1 forward primer |
| | TTCTTAATAGTAAGTATATTAGTATATTACCT | S1_Orf1ab_gene_40_F |
| | GGTTTTCGATCGAAAGATC | |
| 57 | GGGAGTAGACAATTCTAGTCTTCAGAGAGTA | S1 reverse primer |
| | ATATACCACGAAAGACCTTTTCAGGTCTGTG | S1_Orf1ab_gene_40_R |
| | CTCTTTCGAGCACAATC | |
| 58 | GTATAATACGACTCACTATAGGGAATTAGGT | S2 forward primer |
| | TTCTTAATAGTAAGTAATATAAGAAACACACG | S2_Orf1ab_gene_40_F |
| | AATCAGAGAA | |
| 59 | GGGAGTAGACAATTCTAGTCGTATATTATGT | S2 reverse primer |
| | TTCTCTGGTTGACTTCTCTTCTGCG | S2_Orf1ab_gene_40_R |
| 60 | GTATAATACGACTCACTATAGGGATTTTACA | S1 forward primer |
| | AGACTCACGTTAACAATATTGCAGTATATTA | S1_E_gene_60_F |
| | GTATATTACCTGGTTTTCGATCGAAAGATC | |
| 61 | GGGACTGCGCTTCGATTGTGTGCGTACTGC | S1 reverse primer |
| | TTCAGAGAGTAATATACCACGAAAGACCTTT | S1_E_gene_60_R |
| | TCAGGTCTGTGCTCTTTCGAGCACAATC | |
| 62 | GTATAATACGACTCACTATAGGGATTTTACA | S2 forward primer |
| | AGACTCACGTTAACAATATTGCAGTAATATAA | S2_E_gene_60_F |
| | GAAACACACGAATCAGAGAA | |
| 63 | GGGACTGCGCTTCGATTGTGTGCGTACTGC | S2 reverse primer |
| | GTATATTATGTTTCTCTGGTTGACTTCTCTTC | S2_E_gene_60_R |
| | TGCG | |
| 64 | GCAGCCATTAGTATATTAGTATATTACCTGG | S1 forward primer for |
| | TTTTCGATCGAAAGATC | S1_Orf1ab_gene_20_F |
| | | without T7 promoter |
| | | sequence |
| 65 | GCAGCCATTAGTAATATAAGAAACACACGAA | S2 forward primer |
| | TCAGAGAA | S2_Orf1ab_gene_20_F |
| | | without T7 promoter |
| | | sequence |
| 66 | ATTAGGTTTCTTAATAGTAAGTATATTAGTAT | S1 forward primer |
| | ATTACCTGGTTTTCGATCGAAAGATC | S1_Orf1ab_gene_40_F |
| | | without T7 promoter |
| | | sequence |
| 67 | ATTAGGTTTCTTAATAGTAAGTAATATAAGAA | S2 forward primer |
| | ACACACGAATCAGAGAA | S2_Orf1ab_gene_40_F |
| | | without T7 promoter |
| | | sequence |
| 68 | TTTTACAAGACTCACGTTAACAATATTGCAGT | S1 forward primer |
| | ATATTAGTATATTACCTGGTTTTCGATCGAAA | S1_E_gene_60_F without T7 |
| | GATC | promoter sequence |
| 69 | TTTTACAAGACTCACGTTAACAATATTGCAGT | S2 forward primer |
| | AATATAAGAAACACACGAATCAGAGAA | S2_E_gene_60_F without T7 |
| | | promoter sequence |
| 70 | GGGAAGUCAAACCAAGGGAAGUGGUAUAU | S1 strand of dme-ban-5p |
| | UACCUGGUUUUCGAUCGAAAGAUCGACGA | without toehold |
| | 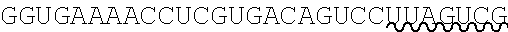 | |
| | 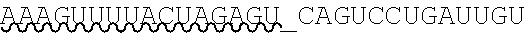 | |
| | GCUCGAAAGAGCACAGACCUGAAAAGGUCU | |
| | UUCGUGGUAUAUUACCUGGUCCCAUCGAA | |
| | AACCGG | |
| 71 | GGGAAGUCAAACCAAGGGACCAGAGAAAC | S2 strand of dme-ban-5p |
| | 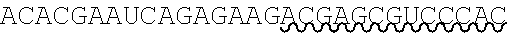 | without toehold |
| | 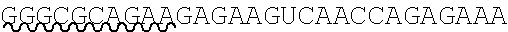 | |
| | CACACUUCCCAUCGAAAACCGG | |
| 72 | GGGAAACUAUACAAUGGGAAGUG | S1 strand of hsa-let-7f-5p |
| | GUAUAUUACCUGGUUUUCGAUCGAAAGAUC | without toehold |
| | 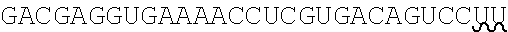 | |
| | 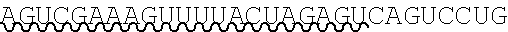 | |
| | AUUGUGCUCGAAAGAGCACAGACCUGAAAA | |
| | GGUCUUUCGUGGUAUAUUAC | |
| | CUGGUCCCCUACUACCUCA | |
| 73 | GGGAAACUAUACAAUGGGACCAGAGAAAC | S2 strand of hsa-let-7f-5p |
| | 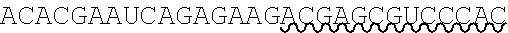 | without toehold |
| | 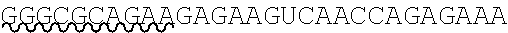 | |
| | CACACUUCCCCUACUACCUCA | |
| 74 | GCAGCCAUUAGGGAAGUGGUAUAUUACCU | S1 strand of |
| | GGUUUUCGAUCGAAAGAUCGACGAGGUGA | Orf1ab_gene_20nt without |
| | 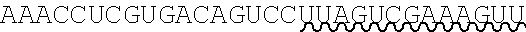 | toehold |
| | 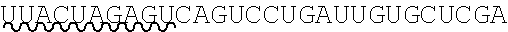 | |
| | AAGAGCACAGACCUGAAAAGGUCUUUCGUG | |
| | GUAUAUUACCUGGUCCCGAUCUGUCCC | |
| 75 | GCAGCCAUUAGGGACCAGAGAAACACACGA | S2 strand of |
| | 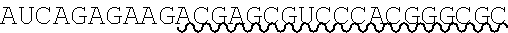 | Orf1ab_gene_20nt without |
| | 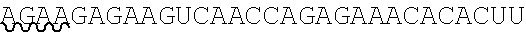 | toehold |
| | CCCGAUCUGUCCC | |
| 76 | AUUAGGUUUCUUAAUAGUAAGGGAAGUG | S1 strand of |
| | GUAUAUUACCUGGUUUUCGAUCGAAAGAUC | Orf1ab_gene_40nt without |
| | 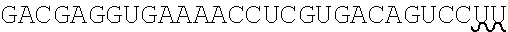 | toehold |
| | 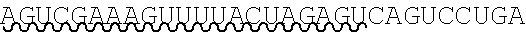 | |
| | UUGUGCUCGAAAGAGCACAGACCUGAAAAG | |
| | GUCUUUCGUGGUAUAUUACCUGGUCCC | |
| | GACUAGAAUUGUCUACUCCC | |
| 77 | AUUAGGUUUCUUAAUAGUAAGGGACCAGAG | S2 strand of |
| | 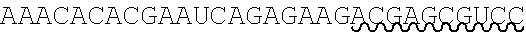 | Orf1ab_gene_40nt without |
| | 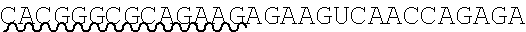 | toehold |
| | AACACACUUCCCGACUAGAAUUGUCUACUC | |
| | CC | |
| 78 | UUUUACAAGACUCACGUUAACAAUAUUGCA | S1 strand of E_gene_60nt |
| | GGGAAGUGGUAUAUUACCUGGUUUUCGAU | without toehold |
| | CGAAAGAUCGACGAGGUGAAAACCUCGUGA | |
| | 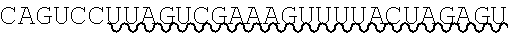 | |
| | CAGUCCUGAUUGUGCUCGAAAGAGCACAG | |
| | ACCUGAAAAGGUCUUUCGUGGUAUAUUACC | |
| | UGGUCCCGCAGUACGCACACAAUCGAAGC | |
| | GCAGUCCC | |
| 79 | UUUUACAAGACUCACGUUAACAAUAUUGCA | S2 strand of E_gene_60nt |
| | _GGGACCAGAGAAACACACGAAUCAGAGAA | without toehold |
| | 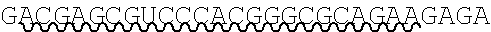 | |
| | AGUCAACCAGAGAAACACACUUCCCGCAG | |
| | UACGCACACAAUCGAAGCGCAGUCCC | |
| 80 | (n)ₐGGGAAGUGGUAUAUUACCUGGUUUUCG | An exemplary S1 sequence |
| | AUCGAAAGAUCGACGAGGUGAAAACCUCGU | (without toeholds). (n)ₓ |
| | GACAGUCC(n)ₓCAGUCCUGAUUGUGCUCGA | represents a region partially |
| | AAGAGCACAGACCUGAAAAGGUCUUUCGUG | complementary to the |
| | GUAUAUUACCUGGUCCC(n)_{b} | releasable RNA segment on |
| | | S1, and (n)ₐ and (n)_{b} |
| | | represent sequences |
| | | complementary to a first and a |
| | | second target RNA |
| | | molecules. |
| 81 | GGGAAGUGGUAUAUUACCUGGUUUUCGAU | Portion of SEQ ID NO.: 80 |
| | CGAAAGAUCGACGAGGUGAAAACCUCGUGA | between (n)ₐ (the first |
| | CAGUCC | sequence complementary to |
| | | the target RNA molecule and |
| | | (n)ₓ (the releasable RNA |
| | | segment). |
| 82 | CAGUCCUGAUUGUGCUCGAAAGAGCACAG | Portion of SEQ ID NO.: 80 |
| | ACCUGAAAAGGUCUUUCGUGGUAUAUUACC | between (n)ₓ (the cleavage |
| | UGGUCCC | product) and (n)_{b} (the second |
| | | sequence complementary to |
| | | the target RNA molecule). |
| 83 | (n)ₐGGGACCAGAGAAACACACGAAUCAGAGA | An exemplary S2 sequence |
| | AG(n)ₓGAGAAGUCAACCAGAGAAACACACUU | (without toeholds). (n)ₓ |
| | CCC(n)_{b} | represents a region partially |
| | | complementary to the |
| | | releasable RNA segment on |
| | | S1, and (n)ₐ and (n)_{b} |
| | | represent sequences |
| | | complementary to the second |
| | | and first target RNA |
| | | molecules. |
| 84 | GGGACCAGAGAAACACACGAAUCAGAGAAG | Portion of SEQ ID NO.: 83 |
| | | between (n)ₐ (the sequence |
| | | complementary to the second |
| | | target RNA molecule and (n)ₓ |
| | | (the region partially |
| | | complementary to the |
| | | releasable RNA segment). |
| 85 | GAGAAGUCAACCAGAGAAACACACUUCCC | Portion of SEQ ID NO.: 83 |
| | | between (n)ₓ (the region |
| | | partially complementary to the |
| | | releasable RNA segment) and |
| | | (n)_{b} (the sequence |
| | | complementary to the second |
| | | target RNA molecule). |
| 86 | GGGAAGUCAAACCAAGUAUAUUAGUAUAU | Tban5p_Cban3pR_single |
| | UACCUGGUUUUCGAUCGAAAGAUCGACGA | design1_full_length |
| | 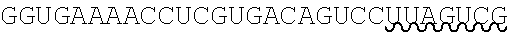 | *single line: sequence |
| | 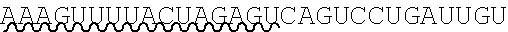 | complementary to target RNA |
| | GAUCGAAAGAGCACAGACCUGAAAAGGUCU | *wavy underline: releasable |
| | 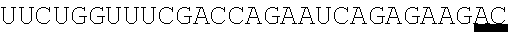 | RNA segment |
| | 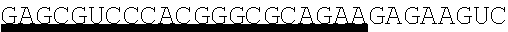 | *bold underline: sequence |
| | AACCAGAGAAACAUAAUAUACAUCGAAAAC | partially complementary to the |
| | CGG | releasable RNA segment |
| 87 | GGGAAGUCAAACCAAGUAUAUUAGUAUAU | Tban5p_Cban3pR_ |
| | UACCUGGUUUUCGAUCGAAAGAUCGACGA | singledesign2_full_length |
| | 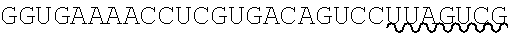 | *single line: sequence |
| | 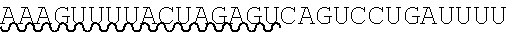 | complementary to target RNA |
| | 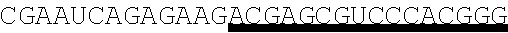 | molecule |
| | 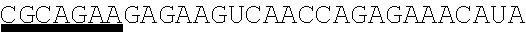 | *wavy underline: releasable |
| | AUAUACAUCGAAAACCGG | RNA segment |
| | | *bold underline: sequence |
| | | partially complementary to the |
| | | releasable RNA segment |
| 88 | (n)ₐGUAUAUUAGUAUAUUACCUGGUUUUCG | An exemplary sequence of a |
| | AUCGAAAGAUCGACGAGGUGAAAACCUCGU | single strand ribozyme. (n)ₐ |
| | GACAGUCC(n)ₓCAGUCCUGAUUGUGAUCGA | and (n)_{b} represent sequences |
| | AAGAGCACAGACCUGAAAAGGUCUUUCUGG | complementary to the first and |
| | UUUCGACCAGAAUCAGAGAAG | second region of the target |
| | (n)_{y}GAGAAGUCAACCAGAGAAACAUAAUAUA | RNA molecule; (n)ₓ |
| | C(n)_{b} | represents a releasable RNA |
| | | segment, and (n)_{y} represents a |
| | | sequence optionally |
| | | complementary to the |
| | | releasable RNA segment |
| 89 | GUAUAUUAGUAUAUUACCUGGUUUUCGAUC | Portion of SEQ ID NO.: 88 |
| | GAAAGAUCGACGAGGUGAAAACCUCGUGAC | between (n)ₐ and (n)ₓ |
| | AGUCC | |
| 90 | CAGUCCUGAUUGUGAUCGAAAGAGCACAGA | Portion of SEQ ID NO.: 88 |
| | CCUGAAAAGGUCUUUCUGGUUUCGACCAG | between (n)ₓ and (n)_{y} |
| | AAUCAGAGAAG | |
| 91 | GAGAAGUCAACCAGAGAAACAUAAUAUAC | Portion of SEQ ID NO.: 88 |
| | | between (n)_{y} and (n)_{b} |
| 92 | GGGAAGUCAAACCAAGUAUAUUAGUAUAU | S1_Tban5p_Cban3pR_Dup |
| | UACCUGGUUUUCGAUCGAAAGAUCGACGA | |
| | 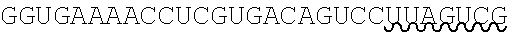 | |
| | 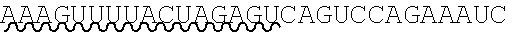 | |
| | UCCCUCACUUCGGUGAGGGAGAUUUCUGA | |
| | GAAGACACCAGAGAAACAUAAUAUAC | |
| | AUCGAAAACCGG | |
| 93 | GGGAAGUCAAACCAAGUAUAUUAGUAUAU | S2_Tban5p_Cban3pR_Dup |
| | UACCUGGUGUCCCACGGGCGCAGAAGAGA | |
| | AGUCAACCAGAGAAACAUAAUAUACAUCGA | |
| | AAACCGG | |
| 94 | GGGAAACUAUACAAUGUAUAUUAGUAUAU | S1_Tlet7f_Cban3pR_Dup |
| | UACCUGGUUUUCGAUCGAAAGAUCGACGA | |
| | 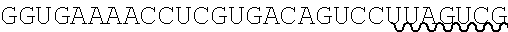 | |
| | 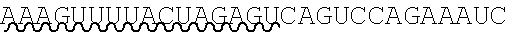 | |
| | UCCCUCACUUCGGUGAGGGAGAUUUCUGA | |
| | GAAGACACCAGAGAAACAUAAUAUACCUAC | |
| | UACCUCA | |
| 95 | GGGAAACUAUACAAUGUAUAUUAGUAUAU | S2_Tlet7f_Cban3pR_Dup |
| | UACCUGGUGUCCCACGGGCGCAGAAGAGA | |
| | AGUCAACCAGAGAAACAUAAUAUACCUACU | |
| | ACCUCA | |
| 96 | GGGAGCAGCCAUUAGUAUAUUAGUAUAUUA | S1_T_Orf1ab_20_Cban3pR_ |
| | CCUGGUUUUCGAUCGAAAGAUCGACGAGG | Dup |
| | 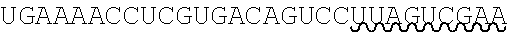 | |
| | 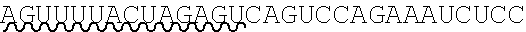 | |
| | CUCACUUCGGUGAGGGAGAUUUCUGAGAA | |
| | GACACCAGAGAAACAUAAUAUACGAUCUGU | |
| | CCC | |
| 97 | GGGAGCAGCCAUUAGUAUAUUAGUAUAUU | S2_T_Orf1ab_20_Cban3pR_ |
| | ACCUGGUGUCCCACGGGCGCAGAAGAGAA | Dup |
| | GUCAACCAGAGAAACAUAAUAUACGAUCUG | |
| | UCCC | |
| 98 | GGGAAUUAGGUUUCUUAAUAGUAAGUAUA | S1_T_Orf1ab_40_Cban3pR_ |
| | UUAGUAUAUUACCUGGUUUUCGAUCGAAAG | Dup |
| | AUCGACGAGGUGAAAACCUCGUGACAGUC | |
| | 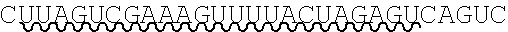 | |
| | CAGAAAUCUCCCUCACUUCGGUGAGGGAG | |
| | AUUUCUGAGAAGACACCAGAGAAACAUAAU | |
| | AUACGACUAGAAUUGUCUACUCCC | |
| 99 | GGGAAUUAGGUUUCUUAAUAGUAAGUAUA | S2_T_Orf1ab_40_Cban3pR_ |
| | UUAGUAUAUUACCUGGUGUCCCACGGGCG | Dup |
| | CAGAAGAGAAGUCAACCAGAGAAACAUAAU | |
| | AUACGACUAGAAUUGUCUACUCCC | |
| 100 | GGGAUUUUACAAGACUCACGUUAACAAUA | S1_T_Egene_60_Cban3pR_ |
| | UUGCAGUAUAUUAGUAUAUUACCUGGUUU | Dup |
| | UCGAUCGAAAGAUCGACGAGGUGAAAACCU | |
| | GGUGAGGGAGAUUUCUGAGAAGACACCAG | |
| | AGAAACAUAAUAUACGCAGUACGCACACAA | |
| | UCGAAGCGCAGUCCC | |
| 101 | GGGAUUUUACAAGACUCACGUUAACAAUA | S2_T_Egene_60_Cban3pR_ |
| | UUGCAGUAUAUUAGUAUAUUACCUGGUGU | Dup |
| | CCCACGGGCGCAGAAGAGAAGUCAACCAGA | |
| | GAAACAUAAUAUACGCAGUACGCACACAAU | |
| | CGAAGCGCAGUCCC | |
| 102 | GUAUAAUACGACUCACUAUAGGGA | RNA T7 promoter sequence |

1. A ribozyme comprising:
a) one or more catalytic domains capable of switching between an active state and an inactive state;
b) one or more releasable RNA segments, wherein each of said releasable RNA segment is flanked by two ribozyme cleavage sites, wherein each cleavage site is cleaved by at least one of the one or more catalytic domains in an active state;
c) one or more target-binding domains, each of which is for the binding of a target RNA molecule;
wherein each of the one or more catalytic domains is linked to one of the one or more target-binding domains;
wherein the catalytic domain is in an inactive state when the target-binding domain linked to said catalytic domain is not bound by the target RNA molecule, and wherein the catalytic domain is in an active state when the target-binding domain linked to said catalytic domain is bound by the target RNA molecule; and
wherein when both cleavage sites flanking a releasable RNA segment are cleaved by the one or more catalytic domains, the one or more releasable RNA segment is released from the ribozyme.

2. The ribozyme according to claim 1, further comprising one or more inhibitory domains;
wherein at least one of the one or more catalytic domains is linked to one of the one or more inhibitory domains, wherein the catalytic domain is in an inactive state due to inhibition from the inhibitory domain, said inhibitory domain being linked to one of the one or more target-binding domains;
wherein when one of the one or more target-binding domains is bound to the target RNA molecule, the inhibitory domain linked to said target-binding domain ceases to inhibit the catalytic domain linked to said inhibitory domain, which results in the catalytic domain switching to an active state.

3. The ribozyme of claim 1, wherein the ribozyme comprises one catalytic domain and one target binding domain, wherein the ribozyme comprises the following structure: 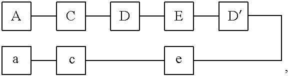
wherein [A] to [a] is in the 5′ to 3′ directionality or the 3′ to 5′ directionality, and wherein:
motifs [A] and [a] constitute the target-binding domain for binding the target RNA molecule;
motifs [C] and [c] constitute the catalytic domain;
motif [D] comprises the first cleavage site capable of being cleaved by the catalytic domain,
motif [D′] comprises the second cleavage site capable of being cleaved by the catalytic domain;
motif [E] comprises a releasable RNA segment;
motif [e] comprises a sequence which is optionally complementary to the sequence of motif [E];
each of the horizontal lines connecting the motifs represents an optional linker region;
and
wherein the catalytic domain is in an active state when the target-binding domain is bound to the target RNA molecule.

4. The ribozyme of claim 2, wherein the ribozyme comprises one catalytic domain, one inhibitory domain, and one target binding domain, wherein the ribozyme comprises an RNA strand with the following structure: 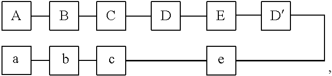
wherein [A] to [a] is in the 5′ to 3′ directionality or the 3′ to 5′ directionality, and wherein:
motifs [A] and [a] constitute the target-binding domain for binding the target RNA molecule;
motifs [B] and [b] constitute the inhibitory domain;
motifs [C] and [c] constitute the first catalytic domain;
motif [D] comprises the first cleavage site capable of being cleaved by the catalytic domain;
motif [D′] comprises the second cleavage site capable of being cleaved by the catalytic domain;
motif [E] comprises a releasable RNA segment;
motif [e] comprises a sequence which is optionally complementary to the sequence of motif [E];
each of the horizontal lines connecting the motifs represents an optional linker region; and
wherein the inhibitory domain is characterized by i) or ii) below:
i) motif [b] anneals with [C] when the target-binding domain is not bound by the target RNA molecule, but anneals with [B] when the target-binding domain is bound by the target RNA molecule,
ii) motif [B] anneals with motif [c] when the first target-binding domain is not bound by the target RNA molecule, but anneals with [b] when the target-binding domain is bound by the target RNA molecule;
wherein the catalytic domain is in an active state when motif [b] is annealed with motif [B].

5. The ribozyme of claim 1, wherein the ribozyme comprises two catalytic domains, optionally wherein the ribozyme comprises two target-binding domains.

6. (canceled)

7. The ribozyme of claim 5, wherein the ribozyme comprises a first RNA strand and a second RNA strand.

8. The ribozyme of claim 7, wherein the ribozyme comprises the following structure: 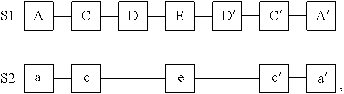
wherein:
S1 is the first RNA strand and S2 is the second RNA strand, wherein [A] to [A′] and [a] to [a′] represent opposite directionalities; and
motifs [A] and [a] constitute a first target-binding domain for binding a first target RNA molecule;
motifs [C] and [c] constitute a first catalytic domain;
motif [D] comprises a first cleavage site capable of being cleaved by the first catalytic domain;
motifs [A′] and [a′] constitute a second target-binding domain for binding a second target RNA molecule;
motifs [C′] and [c′] constitute a second catalytic domain;
motif [D′] comprises a second cleavage site capable of being cleaved by the second catalytic domain;
motif [E] comprises a releasable RNA segment;
motif [e] comprises a sequence which is optionally complementary to the sequence of motif [E];
each of the horizontal lines connecting the motifs represents an optional linker region; and
wherein the first catalytic domain is in an active state when the first target-binding domain is bound to first target RNA molecule; and wherein the second catalytic domain is in an active state when the second target-binding domain is bound to the second target RNA molecule.

9. The ribozyme of claim 2, wherein the ribozyme comprises the following structure: 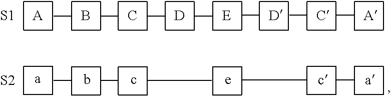
wherein:
S1 is the first RNA strand and S2 is the second RNA strand, wherein [A] to [A′] and [a] to [a′] represent opposite directionalities; wherein
motifs [A] and [a] constitute a first target-binding domain for binding a first target RNA molecule;
motifs [B] and [b] constitute a inhibitory domain;
motifs [C] and [c] constitute a first catalytic domain;
motif [D] comprises a first cleavage site for being cleaved by the first catalytic domain;
motifs [A′] and [a′] constitute a second target-binding domain for binding a second target RNA molecule;
motifs [C′] and [c′] constitute a second catalytic domain;
motif [D′] comprises a second cleavage site capable of being cleaved by the second catalytic domain;
motif [E] comprises a releasable RNA segment;
motif [e] comprises a sequence which is optionally complementary to the sequence of motif [E];
each of the horizontal lines connecting the motifs represents an optional linker region;
wherein the inhibitory domain is characterized by i) or ii) below:
i) motif [b] anneals with [C] when the first target-binding domain is not bound by the first target RNA molecule, but anneals with [B] when the first target-binding domain is bound by the first target RNA molecule,
ii) motif [B] anneals with motif [c] when the first target-binding domain is not bound by the first target RNA molecule, but anneals with [b] when the first target-binding domain is bound by the first target RNA molecule;
wherein the first catalytic domain is in an active state when motif [b] is annealed with motif [B].

10. The ribozyme of claim 4, wherein the inhibitory domain is further characterized by i) or ii) below:
i) motif [b] is at least 50% complementary to motif [C], and at least 20% complementary to motif [B], and
ii) motif [B] is at least 50% complementary to motif [c], and at least 20% complementary to motif [b].

11. The ribozyme of claim 2, wherein the ribozyme comprises the following structure: 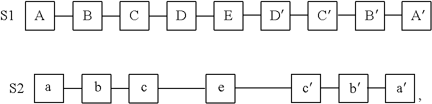
wherein:
S1 is the first RNA strand and S2 is the second RNA strand, wherein [A] to [A′] and [a] to [a′] represent opposite directionalities; wherein
motifs [A] and [a] constitute a first target-binding domain for binding a first target RNA molecule;
motifs [B] and [b] constitute a first inhibitory domain;
motifs [C] and [c] constitute a first catalytic domain;
motif [D] comprises a first cleavage site capable of being cleaved by the first catalytic domain;
motifs [A′] and [a′] constitute a second target-binding domain for binding a second target RNA molecule;
motifs [B′] and [b′] constitute a second inhibitory domain;
motifs [C′] and [c′] constitute a second catalytic domain;
motif [D′] comprises a second cleavage site capable of being cleaved by the second catalytic domain;
motif [E] comprises a releasable RNA segment;
motif [e] comprises a sequence which is optionally complementary to the sequence of motif [E];
each of the horizontal lines connecting the motifs represents an optional linker region;
wherein the first inhibitory domain is characterized by i) or ii) below:
i) motif [b] anneals with [C] when the first target-binding domain is not bound by the first target RNA molecule, but anneals with [B] when the first target-binding domain is bound by the first target RNA molecule,
ii) motif [B] anneals with motif [c] when the first target-binding domain is not bound by the first target RNA molecule, but anneals with [b] when the first target-binding domain is bound by the first target RNA molecule,
wherein the second inhibitory domain is characterized by i) or ii) below:
i) motif [b′] anneals with motif [C′] when the second target-binding domain is not bound by the first target RNA molecule, but anneals with [B′] when the second target-binding domain is bound by the second target RNA molecule,
ii) motif [B′] anneals with motif [c′] when the second target-binding domain is not bound by the second target RNA molecule, but anneals with [b′] when the second target-binding domain is bound by the second target RNA molecule,
wherein the first catalytic domain is in an active state when motif [b] is annealed with motif [B], and the second catalytic domain is in an active state when motif [b′] is annealed with motif [B′].

12. The ribozyme of claim 11, wherein the first inhibitory domain is further characterized by i) or ii) below:
i) motif [b] is at least 50% complementary to motif [C], and at least 20% complementary to motif [B], and
ii) motif [B] is at least 50% complementary to motif [c], and at least 20% complementary to motif [b]; and
the second inhibitory domain is further characterized by i) or ii) below:
i) motif [b′] is at least 50% complementary to motif [C′], and at least 20% complementary to motif [B′],
ii) motif [B′] is at least 50% complementary to motif [c′], and at least 20% complementary to motif [b′].

13. The ribozyme of claim 3, wherein the optional linker regions individually or collectively form one or more secondary structures.

14. The ribozyme of claim 13, wherein the one or more secondary structures are selected from the group consisting of: single-nucleotide bulges, three-nucleotide bulges, stems, stem loops, t-RNA type structures, cloverleaves, tetraloops, pseudoknots, symmetrical internal loops, asymmetrical internal loops, three stem junctions (3-way junctions), four stem junctions (4-way junctions), two-stem junctions (2-way junctions) or coaxial stacks or combinations thereof.

15. The ribozyme complex of claim 3, wherein motifs [E] and [e] are complementary to each other with a complementarity of less than 70%.

16. The ribozyme of claim 11, wherein the first RNA strand (S1) and second RNA strand (S2) comprise the sequences of SEQ ID NO.: 26 and SEQ ID NO.: 29 respectively.

17. The ribozyme of claim 1, wherein the target RNA molecule is more than 16 nucleotides in length, or wherein the target RNA molecule comprises a region which is complementary to the target-binding domain, wherein said region is more than 16 nucleotides in length, or wherein the releasable RNA segment is 6 to 150 nucleotides in length.

18.-19. (canceled)

20. The ribozyme of claim 1, wherein the one or more target-binding domains are for binding the same target RNA molecule.

21. The ribozyme of claim 1, wherein the releasable RNA segment comprises a sequence that is identical to at least one of the one or more target RNA molecules.

22.-32. (canceled)

33. A polynucleotide encoding the ribozyme of claim 1.

34. A kit comprising the ribozyme of claim 1.

35.-37. (canceled)


SEQUENCE LISTING (102 sequences)

SEQ ID NO: 1 - RNA, 23 nt, Artificial Sequence
  Sequence of exemplary target RNA dme-bantam-5p
ccgguuuucgauuugguuugacu

SEQ ID NO: 2 - RNA, 181 nt, Artificial Sequence
  Resulting S1 strand for dme-ban-5p ribozyme (S1_Tban5p_Cban3pR)
gggaagucaaaccaaguauauuaguauauuaccugguuuucgaucgaaagaucgacgaggugaaaaccucgugacaguccuuagucgaaaguuuuacuagagucaguccugauugugcucgaaagagcacagaccugaaaaggucuuucgugguauauuacucucugaaaucgaaaaccgg

SEQ ID NO: 3 - RNA, 109 nt, Artificial Sequence
  Resulting S2 strand for dme-ban-5p ribozyme (S2_Tban5p_Cban3pR)
gggaagucaaaccaaguaauauaagaaacacacgaaucagagaagacgagcgucccacgggcgcagaagagaagucaaccagagaaacauaauauacaucgaaaaccgg

SEQ ID NO: 4 - RNA, 22 nt, Artificial Sequence
  Sequence of exemplary target RNA hsa-let-7f-5p
ugagguaguagauuguauaguu

SEQ ID NO: 5 - RNA, 180 nt, Artificial Sequence
  Resulting S1 strand for hsa-let-7f-5p ribozyme (S1_Tlet7f_Cban3pR)
gggaaacuauacaauguauauuaguauauuaccugguuuucgaucgaaagaucgacgaggugaaaaccucgugacaguccuuagucgaaaguuuuacuagagucaguccugauugugcucgaaagagcacagaccugaaaaggucuuucgugguauauuacucucugaacuacuaccuca

SEQ ID NO: 6 - RNA, 108 nt, Artificial Sequence
  Resulting S2 strand for hsa-let-7f-5p ribozyme (S2_Tlet7f_Cban3pR)
gggaaacuauacaauguaauauaagaaacacacgaaucagagaagacgagcgucccacgggcgcagaagagaagucaaccagagaaacauaauauaccuacuaccuca

SEQ ID NO: 7 - DNA, 24 bp, Artificial Sequence
  T7 promoter sequence
gtataatacgactcactataggga

SEQ ID NO: 8 - RNA, 156 nt, Artificial Sequence
  S1 sequence with an exemplary cleavage product, and (n)a and (n)b represent sequences complementary to the target RNA molecule.
  misc_feature (1)..(1): (n)a represents the first sequence complementary to the target RNA molecule.
  misc_feature (63)..(91): Exemplary cleavage product SEQ ID NO. 25
  misc_feature (156)..(156): (n)b represents the second sequence complementary to the target RNA molecule.
nguauauuaguauauuaccugguuuucgaucgaaagaucgacgaggugaaaaccucgugacaguccuuagucgaaaguuuuacuagagucaguccugauugugcucgaaagagcacagaccugaaaaggucuuucgugguauauuacucucugaan

SEQ ID NO: 9 - RNA, 84 nt, Artificial Sequence
  S2 sequence. (n)a and (n)b represent sequences complementary to the target  RNA molecule.
  misc_feature (1)..(1): (n)a represents the first sequence complementary to the target RNA molecule.
  misc_feature (84)..(84): (n)b represents the second sequence complementary to the target RNA molecule.
nguaauauaagaaacacacgaaucagagaagacgagcgucccacgggcgcagaagagaagucaaccagagaaacauaauauacn

SEQ ID NO: 10 - DNA, 98 bp, Artificial Sequence
  Core S1 DNA template
ctggttttcgatcgaaagatcgacgaggtgaaaacctcgtgacagtccttagtcgaaagttttactagagtcagtcctgattgtgctcgaaagagcac

SEQ ID NO: 11 - DNA, 54 bp, Artificial Sequence
  Core S2 DNA template
agaaacacacgaatcagagaagacgagcgtcccacgggcgcagaagagaagtca

SEQ ID NO: 12 - RNA, 12 nt, Artificial Sequence
  Reverse complement of 5′ paired sequence of dme-ban-5p
aucgaaaaccgg

SEQ ID NO: 13 - RNA, 11 nt, Artificial Sequence
  Reverse complement of 3′ paired sequence of dme-ban-5p
agucaaaccaa

SEQ ID NO: 14 - DNA, 73 bp, Artificial Sequence
  S1 forward primer for dme-ban-5p
  promoter (1)..(24): T7 promoter sequence
gtataatacgactcactatagggaagtcaaaccaagtatattagtatattacctggttttcgatcgaaagatc

SEQ ID NO: 15 - DNA, 71 bp, Artificial Sequence
  S1 reverse primer for dme-ban-5p
ccggttttcgatttcagagagtaatataccacgaaagaccttttcaggtctgtgctctttcgagcacaatc

SEQ ID NO: 16 - DNA, 64 bp, Artificial Sequence
  S2 forward primer for dme-ban-5p
  promoter (1)..(24): T7 promoter sequence
gtataatacgactcactatagggaagtcaaaccaagtaatataagaaacacacgaatcagagaa

SEQ ID NO: 17 - DNA, 48 bp, Artificial Sequence
  S2 reverse primer for dme-ban-5p
ccggttttcgatgtatattatgtttctctggttgacttctcttctgcg

SEQ ID NO: 18 - RNA, 11 nt, Artificial Sequence
  Reverse complement of 5′ paired sequence of hsa-let-7f-5p
cuacuaccuca

SEQ ID NO: 19 - RNA, 11 nt, Artificial Sequence
  Reverse complement of 3′ paired sequence of hsa-let-7f-5p
aacuauacaau

SEQ ID NO: 20 - DNA, 73 bp, Artificial Sequence
  S1 forward primer for hsa-let-7f-5p
  promoter (1)..(24): T7 promoter sequence
gtataatacgactcactatagggaaactatacaatgtatattagtatattacctggttttcgatcgaaagatc

SEQ ID NO: 21 - DNA, 70 bp, Artificial Sequence
  S1 reverse primer for hsa-let-7f-5p
tgaggtagtagttcagagagtaatataccacgaaagaccttttcaggtctgtgctctttcgagcacaatc

SEQ ID NO: 22 - DNA, 64 bp, Artificial Sequence
  S2 forward primer for hsa-let-7f-5p
  promoter (1)..(24): T7 promoter sequence
gtataatacgactcactatagggaaactatacaatgtaatataagaaacacacgaatcagagaa

SEQ ID NO: 23 - DNA, 47 bp, Artificial Sequence
  S2 reverse primer for hsa-let-7f-5p
tgaggtagtaggtatattatgtttctctggttgacttctcttctgcg

SEQ ID NO: 24 - RNA, 22 nt, Artificial Sequence
  hsa-miR-122-5p, a control RNA molecule with a dissimilar sequence to a target RNA molecule to test sequence specificity of the ribozymes
uggagugugacaaugguguuug

SEQ ID NO: 25 - RNA, 29 nt, Artificial Sequence
  Exemplary cleavage product of SEQ ID NO. 8.
guccuuagucgaaaguuuuacuagaguca

SEQ ID NO: 26 - RNA, 134 nt, Artificial Sequence
  An exemplary  S1 sequence with toeholds. (n)x represents a releasable RNA segment, and (n)a and (n)b represent sequences complementary to the first and second target  RNA molecules.
  misc_feature (1)..(1): (n)a represents the first sequence complementary to the target RNA molecule.
  misc_feature (67)..(67): (n)x represents a releasable RNA segment.
  misc_feature (134)..(134): (n)b represents the second sequence complementary to the target RNA molecule.
nguauauuaguauauuaccugguuuucgaucgaaagaucgacgaggugaaaaccucgugacaguccncaguccugauugugcucgaaagagcacagaccugaaaaggucuuucgugguauauuacucucugaan

SEQ ID NO: 27 - RNA, 65 nt, Artificial Sequence
  Portion of SEQ ID NO. 26 between (n)a (the first sequence complementary to the target  RNA molecule and (n)x (the releasable RNA segment).
guauauuaguauauuaccugguuuucgaucgaaagaucgacgaggugaaaaccucgugacagucc

SEQ ID NO: 28 - RNA, 66 nt, Artificial Sequence
  Portion of SEQ ID NO. 26 between (n)x (the cleavage product) and (n)b (the second sequence complementary to the target RNA molecule).
caguccugauugugcucgaaagagcacagaccugaaaaggucuuucgugguauauuacucucugaa

SEQ ID NO: 29 - RNA, 62 nt, Artificial Sequence
  An exemplary S2 sequence (with toeholds). (n)x represents a region partially complementary to the releasable RNA segment on S1, and (n)a and (n)b represent sequences complementary to the second and first target RNA molecules.
  misc_feature (1)..(1): (n)a represents the first sequence complementary to the target RNA molecule.
  misc_feature (32)..(32): (n)x represents a region partially complementary to the releasable RNA segment on S1.
  misc_feature (62)..(62): (n)b represents the second sequence complementary to the target RNA molecule.
nguaauauaagaaacacacgaaucagagaagngagaagucaaccagagaaacauaauauacn

SEQ ID NO: 30 - RNA, 30 nt, Artificial Sequence
  Portion of SEQ ID NO. 29 between (n)a (the sequence complementary to the second target RNA molecule and (n)x (the region partially complementary to the releasable RNA segment).
guaauauaagaaacacacgaaucagagaag

SEQ ID NO: 31 - RNA, 29 nt, Artificial Sequence
  Portion of SEQ ID NO. 29 between (n)x (the region partially complementary to the releasable RNA segment) and (n)b (the sequence complementary to the second target RNA molecule).
gagaagucaaccagagaaacauaauauac

SEQ ID NO: 32 - RNA, 154 nt, Artificial Sequence
  An exemplary sequence of the region of S1 between motifs [A] and [A′], in the direction of [A] to [A′].
guauauuaguauauuaccugguuuucgaucgaaagaucgacgaggugaaaaccucgugacaguccuuagucgaaaguuuuacuagagucaguccugauugugcucgaaagagcacagaccugaaaaggucuuucgugguauauuacucucugaa

SEQ ID NO: 33 - RNA, 82 nt, Artificial Sequence
  An exemplary sequence of the region of S2 between motifs [a′] and [a], in the direction of [a′] to [a].
guaauauaagaaacacacgaaucagagaagacgagcgucccacgggcgcagaagagaagucaaccagagaaacauaauauac

SEQ ID NO: 34 - RNA, 107 nt, Artificial Sequence
  An exemplary sequence of the region of S1 between motifs [B] and [B′], in the direction of [B] to [B′].
  misc_feature (1)..(9): Catalytic domain
guauauuaccugguuuucgaucgaaagaucgacgaggugaaaaccucgugacaguccuuagucgaaaguuuuacuagagucaguccugauugugcucgaaagagcac

SEQ ID NO: 35 - RNA, 66 nt, Artificial Sequence
  An exemplary sequence of the region of S2 between motifs [b′] and [b], in the direction of [b′] to [b].
  misc_feature (55)..(66): Catalytic domain
agaaacacacgaaucagagaagacgagcgucccacgggcgcagaagagaagucaaccagagaaaca

SEQ ID NO: 36 - DNA, 49 bp, Artificial Sequence
  S1 forward primer for dme-ban-5p without the T7 promoter sequence
agtcaaaccaagtatattagtatattacctggttttcgatcgaaagatc

SEQ ID NO: 37 - DNA, 40 bp, Artificial Sequence
  S2 forward primer for dme-ban-5p without T7 promoter sequence
agtcaaaccaagtaatataagaaacacacgaatcagagaa

SEQ ID NO: 38 - DNA, 49 bp, Artificial Sequence
  S1 forward primer for hsa-let-7f-5p without T7 promoter sequence
aactatacaatgtatattagtatattacctggttttcgatcgaaagatc

SEQ ID NO: 39 - DNA, 40 bp, Artificial Sequence
  S2 forward primer for hsa-let-7f-5p without T7 promoter sequence
aactatacaatgtaatataagaaacacacgaatcagagaa

SEQ ID NO: 40 - RNA, 40 nt, Artificial Sequence
  Target RNA T7_Orf1ab_ 20
  promoter (1)..(24): T7 promoter sequence
guauaauacgacucacuauagggacagaucuaauggcugc

SEQ ID NO: 41 - RNA, 174 nt, Artificial Sequence
  S1_Orf1ab _20
gcagccauuaguauauuaguauauuaccugguuuucgaucgaaagaucgacgaggugaaaaccucgugacaguccuuagucgaaaguuuuacuagagucaguccugauugugcucgaaagagcacagaccugaaaaggucuuucgugguauauuacucucugaagaucuguccc

SEQ ID NO: 42 - RNA, 102 nt, Artificial Sequence
  S2_Orf1ab_20
gcagccauuaguaauauaagaaacacacgaaucagagaagacgagcgucccacgggcgcagaagagaagucaaccagagaaacauaauauacgaucuguccc

SEQ ID NO: 43 - RNA, 60 nt, Artificial Sequence
  Target RNA T7_Orf1ab_ 40
  promoter (1)..(24): T7 promoter sequence
guauaauacgacucacuauagggaguagacaauucuagucuuacuauuaagaaaccuaau

SEQ ID NO: 44 - RNA, 194 nt, Artificial Sequence
  S1_Orf1ab _40
auuagguuucuuaauaguaaguauauuaguauauuaccugguuuucgaucgaaagaucgacgaggugaaaaccucgugacaguccuuagucgaaaguuuuacuagagucaguccugauugugcucgaaagagcacagaccugaaaaggucuuucgugguauauuacucucugaagacuagaauugucuacuccc

SEQ ID NO: 45 - RNA, 122 nt, Artificial Sequence
  S2_Orf1ab_40
auuagguuucuuaauaguaaguaauauaagaaacacacgaaucagagaagacgagcgucccacgggcgcagaagagaagucaaccagagaaacauaauauacgacuagaauugucuacuccc

SEQ ID NO: 46 - RNA, 80 nt, Artificial Sequence
  T7_Egene_60
  promoter (1)..(24): T7 promoter sequence
guauaauacgacucacuauagggacugcgcuucgauugugugcguacugcugcaauauuguuaacgugagucuuguaaaa

SEQ ID NO: 47 - RNA, 214 nt, Artificial Sequence
  S1_E_gene_60
uuuuacaagacucacguuaacaauauugcaguauauuaguauauuaccugguuuucgaucgaaagaucgacgaggugaaaaccucgugacaguccuuagucgaaaguuuuacuagagucaguccugauugugcucgaaagagcacagaccugaaaaggucuuucgugguauauuacucucugaagcaguacgcacacaaucgaagcgcaguccc

SEQ ID NO: 48 - RNA, 142 nt, Artificial Sequence
  S2_Egene_60
uuuuacaagacucacguuaacaauauugcaguaauauaagaaacacacgaaucagagaagacgagcgucccacgggcgcagaagagaagucaaccagagaaacauaauauacgcaguacgcacacaaucgaagcgcaguccc

SEQ ID NO: 49 - RNA, 16 nt, Artificial Sequence
  Target RNA T7_Orf1ab_ 20 without T7 promoter sequence
cagaucuaauggcugc

SEQ ID NO: 50 - RNA, 36 nt, Artificial Sequence
  Target RNA T7_Orf1ab_ 40 without T7 promoter sequence
guagacaauucuagucuuacuauuaagaaaccuaau

SEQ ID NO: 51 - RNA, 56 nt, Artificial Sequence
  T7_Egene_60 without T7 promoter sequence
cugcgcuucgauugugugcguacugcugcaauauuguuaacgugagucuuguaaaa

SEQ ID NO: 52 - DNA, 72 bp, Artificial Sequence
  S1 forward primer for S1_Orf1ab_gene_20_F
  promoter (1)..(24): T7 promoter sequence
gtataatacgactcactatagggagcagccattagtatattagtatattacctggttttcgatcgaaagatc

SEQ ID NO: 53 - DNA, 69 bp, Artificial Sequence
  S1 reverse primer S1_Orf1ab_gene_20_R
gggacagatcttcagagagtaatataccacgaaagaccttttcaggtctgtgctctttcgagcacaatc

SEQ ID NO: 54 - DNA, 63 bp, Artificial Sequence
  S2 forward primer S2_Orf1ab_gene_20_F
  promoter (1)..(24): T7 promoter sequence
gtataatacgactcactatagggagcagccattagtaatataagaaacacacgaatcagagaa

SEQ ID NO: 55 - DNA, 46 bp, Artificial Sequence
  S2 reverse primer S2_Orf1ab_gene_20_R
gggacagatcgtatattatgtttctctggttgacttctcttctgcg

SEQ ID NO: 56 - DNA, 82 bp, Artificial Sequence
  S1 forward primer S1_Orf1ab_gene_40_F
  promoter (1)..(24): T7 promoter sequence
gtataatacgactcactatagggaattaggtttcttaatagtaagtatattagtatattacctggttttcgatcgaaagatc

SEQ ID NO: 57 - DNA, 79 bp, Artificial Sequence
  S1 reverse primer S1_Orf1ab_gene_40_R
gggagtagacaattctagtcttcagagagtaatataccacgaaagaccttttcaggtctgtgctctttcgagcacaatc

SEQ ID NO: 58 - DNA, 73 bp, Artificial Sequence
  S2 forward primer S2_Orf1ab_gene_40_F
  promoter (1)..(24): T7 promoter sequence
gtataatacgactcactatagggaattaggtttcttaatagtaagtaatataagaaacacacgaatcagagaa

SEQ ID NO: 59 - DNA, 56 bp, Artificial Sequence
  S2 reverse primer S2_Orf1ab_gene_40_R
gggagtagacaattctagtcgtatattatgtttctctggttgacttctcttctgcg

SEQ ID NO: 60 - DNA, 92 bp, Artificial Sequence
  S1 forward primer S1_E_gene_60_F
  promoter (1)..(24): T7 promoter sequence
gtataatacgactcactatagggattttacaagactcacgttaacaatattgcagtatattagtatattacctggttttcgatcgaaagatc

SEQ ID NO: 61 - DNA, 89 bp, Artificial Sequence
  S1 reverse primer S1_E_gene_60_R
gggactgcgcttcgattgtgtgcgtactgcttcagagagtaatataccacgaaagaccttttcaggtctgtgctctttcgagcacaatc

SEQ ID NO: 62 - DNA, 83 bp, Artificial Sequence
  S2 forward primer S2_E_gene_60_F
  promoter (1)..(24): T7 promoter sequence
gtataatacgactcactatagggattttacaagactcacgttaacaatattgcagtaatataagaaacacacgaatcagagaa

SEQ ID NO: 63 - DNA, 66 bp, Artificial Sequence
  S2 reverse primer S2_E_gene_60_R
gggactgcgcttcgattgtgtgcgtactgcgtatattatgtttctctggttgacttctcttctgcg

SEQ ID NO: 64 - DNA, 48 bp, Artificial Sequence
  S1 forward primer for S1_Orf1ab_gene_20_F without T7 promoter sequence
gcagccattagtatattagtatattacctggttttcgatcgaaagatc

SEQ ID NO: 65 - DNA, 39 bp, Artificial Sequence
  S2 forward primer S2_Orf1ab_gene_20_F without T7 promoter sequence
gcagccattagtaatataagaaacacacgaatcagagaa

SEQ ID NO: 66 - DNA, 58 bp, Artificial Sequence
  S1 forward primer S1_Orf1ab_gene_40_F without T7 promoter sequence
attaggtttcttaatagtaagtatattagtatattacctggttttcgatcgaaagatc

SEQ ID NO: 67 - DNA, 49 bp, Artificial Sequence
  S2 forward primer S2_Orf1ab_gene_40_F without T7 promoter sequence
attaggtttcttaatagtaagtaatataagaaacacacgaatcagagaa

SEQ ID NO: 68 - DNA, 68 bp, Artificial Sequence
  S1 forward primer S1_E_gene_60_F without T7 promoter sequence
ttttacaagactcacgttaacaatattgcagtatattagtatattacctggttttcgatcgaaagatc

SEQ ID NO: 69 - DNA, 59 bp, Artificial Sequence
  S2 forward primer S2_E_gene_60_F without T7 promoter sequence
ttttacaagactcacgttaacaatattgcagtaatataagaaacacacgaatcagagaa

SEQ ID NO: 70 - RNA, 181 nt, Artificial Sequence
  S1 strand of dme-ban-5p without toehold
gggaagucaaaccaagggaagugguauauuaccugguuuucgaucgaaagaucgacgaggugaaaaccucgugacaguccuuagucgaaaguuuuacuagagucaguccugauugugcucgaaagagcacagaccugaaaaggucuuucgugguauauuaccuggucccaucgaaaaccgg

SEQ ID NO: 71 - RNA, 109 nt, Artificial Sequence
  S2 strand of dme-ban-5p without toehold
gggaagucaaaccaagggaccagagaaacacacgaaucagagaagacgagcgucccacgggcgcagaagagaagucaaccagagaaacacacuucccaucgaaaaccgg

SEQ ID NO: 72 - RNA, 180 nt, Artificial Sequence
  S1 strand of hsa-let-7f-5p without toehold
gggaaacuauacaaugggaagugguauauuaccugguuuucgaucgaaagaucgacgaggugaaaaccucgugacaguccuuagucgaaaguuuuacuagagucaguccugauugugcucgaaagagcacagaccugaaaaggucuuucgugguauauuaccugguccccuacuaccuca

SEQ ID NO: 73 - RNA, 108 nt, Artificial Sequence
  S2 strand of hsa-let-7f-5p without toehold
gggaaacuauacaaugggaccagagaaacacacgaaucagagaagacgagcgucccacgggcgcagaagagaagucaaccagagaaacacacuuccccuacuaccuca

SEQ ID NO: 74 - RNA, 174 nt, Artificial Sequence
  S1 strand of Orf1ab_gene_20nt without toehold
gcagccauuagggaagugguauauuaccugguuuucgaucgaaagaucgacgaggugaaaaccucgugacaguccuuagucgaaaguuuuacuagagucaguccugauugugcucgaaagagcacagaccugaaaaggucuuucgugguauauuaccuggucccgaucuguccc

SEQ ID NO: 75 - RNA, 102 nt, Artificial Sequence
  S2 strand of Orf1ab_gene_20nt without toehold
gcagccauuagggaccagagaaacacacgaaucagagaagacgagcgucccacgggcgcagaagagaagucaaccagagaaacacacuucccgaucuguccc

SEQ ID NO: 76 - RNA, 194 nt, Artificial Sequence
  S1strand of Orf1ab_gene_40nt without toehold
auuagguuucuuaauaguaagggaagugguauauuaccugguuuucgaucgaaagaucgacgaggugaaaaccucgugacaguccuuagucgaaaguuuuacuagagucaguccugauugugcucgaaagagcacagaccugaaaaggucuuucgugguauauuaccuggucccgacuagaauugucuacuccc

SEQ ID NO: 77 - RNA, 122 nt, Artificial Sequence
  S2 strand of Orf1ab_gene_40nt without toehold
auuagguuucuuaauaguaagggaccagagaaacacacgaaucagagaagacgagcgucccacgggcgcagaagagaagucaaccagagaaacacacuucccgacuagaauugucuacuccc

SEQ ID NO: 78 - RNA, 214 nt, Artificial Sequence
  S1 strand of E_gene_60nt without toehold
uuuuacaagacucacguuaacaauauugcagggaagugguauauuaccugguuuucgaucgaaagaucgacgaggugaaaaccucgugacaguccuuagucgaaaguuuuacuagagucaguccugauugugcucgaaagagcacagaccugaaaaggucuuucgugguauauuaccuggucccgcaguacgcacacaaucgaagcgcaguccc

SEQ ID NO: 79 - RNA, 142 nt, Artificial Sequence
  S2 strand of E_gene_60nt without toehold
uuuuacaagacucacguuaacaauauugcagggaccagagaaacacacgaaucagagaagacgagcgucccacgggcgcagaagagaagucaaccagagaaacacacuucccgcaguacgcacacaaucgaagcgcaguccc

SEQ ID NO: 80 - RNA, 134 nt, Artificial Sequence
  An exemplary S1 sequence without toeholds. (n)x represents a region partially complementary to the releasable RNA segment on S1, and (n)a and (n)b represent sequences complementary to the target miRNA.
  misc_feature (1)..(1): (n)a represents the first sequence complementary to the target RNA molecule.
  misc_feature (67)..(67): (n)x represents a region partially complementary to the releasable RNA segment on S1.
  misc_feature (134)..(134): (n)b represents the second sequence complementary to the target RNA molecule.
ngggaagugguauauuaccugguuuucgaucgaaagaucgacgaggugaaaaccucgugacaguccncaguccugauugugcucgaaagagcacagaccugaaaaggucuuucgugguauauuaccuggucccn

SEQ ID NO: 81 - RNA, 65 nt, Artificial Sequence
  Portion of SEQ ID NO. 80 between (n)a (the first sequence complementary to the target  RNA molecule and (n)x (the releasable RNA segment).
gggaagugguauauuaccugguuuucgaucgaaagaucgacgaggugaaaaccucgugacagucc

SEQ ID NO: 82 - RNA, 66 nt, Artificial Sequence
  Portion of SEQ ID NO. 80 between (n)x (the cleavage product) and (n)b (the second sequence complementary to the target RNA molecule).
caguccugauugugcucgaaagagcacagaccugaaaaggucuuucgugguauauuaccugguccc

SEQ ID NO: 83 - RNA, 62 nt, Artificial Sequence
  An exemplary S2 sequence (without toeholds). (n)x represents a region partially complementary to the releasable RNA segment on S1, and (n)a and (n)b represent sequences complementary to the second and first target RNA molecules.
  misc_feature (1)..(1): (n)a represents the first sequence complementary to the target RNA molecule.
  misc_feature (32)..(32): (n)x represents a region partially complementary to the releasable RNA segment on S1.
  misc_feature (62)..(62): (n)b represents the second sequence complementary to the target RNA molecule.
ngggaccagagaaacacacgaaucagagaagngagaagucaaccagagaaacacacuucccn

SEQ ID NO: 84 - RNA, 30 nt, Artificial Sequence
  Portion of SEQ ID NO. 83 between (n)a (the sequence complementary to the second target RNA molecule and (n)x (the region partially complementary to the releasable RNA segment).
gggaccagagaaacacacgaaucagagaag

SEQ ID NO: 85 - RNA, 29 nt, Artificial Sequence
  Portion of SEQ ID NO. 83  between (n)x (the region partially complementary to the releasable RNA segment) and (n)b (the sequence complementary to the second target RNA molecule).
gagaagucaaccagagaaacacacuuccc

SEQ ID NO: 86 - RNA, 237 nt, Artificial Sequence
  Tban5p_Cban3pR_singledesign1_full_length
  misc_feature (5)..(15): sequence complementary to target RNA
  misc_feature (81)..(103): Releasable RNA segment
  misc_feature (174)..(196): Sequence partially complementary to the releasable RNA segment
  misc_feature (226)..(237): Sequence complementary to target RNA
gggaagucaaaccaaguauauuaguauauuaccugguuuucgaucgaaagaucgacgaggugaaaaccucgugacaguccuuagucgaaaguuuuacuagagucaguccugauugugaucgaaagagcacagaccugaaaaggucuuucugguuucgaccagaaucagagaagacgagcgucccacgggcgcagaagagaagucaaccagagaaacauaauauacaucgaaaaccgg

SEQ ID NO: 87 - RNA, 193 nt, Artificial Sequence
  Tban5p_Cban3pR_singledesign2_full_length
  misc_feature (5)..(15): Sequence complementary to target RNA molecule
  misc_feature (81)..(103): Releasable RNA segment
  misc_feature (130)..(152): Sequence partially complementary to the releasable RNA segment
  misc_feature (182)..(193): Sequence complementary to target RNA molecule
gggaagucaaaccaaguauauuaguauauuaccugguuuucgaucgaaagaucgacgaggugaaaaccucgugacaguccuuagucgaaaguuuuacuagagucaguccugauuuucgaaucagagaagacgagcgucccacgggcgcagaagagaagucaaccagagaaacauaauauacaucgaaaaccgg

SEQ ID NO: 88 - RNA, 168 nt, Artificial Sequence
  An exemplary sequence of a single strand ribozyme.
  misc_feature (1)..(1): (n)a represent sequences complementary to the first region  of the target  RNA molecule
  misc_feature (67)..(67): (n)x represents a releasable RNA segment
  misc_feature (138)..(138): (n)y represents a sequence optionally complementary to the releasable RNA segment
  misc_feature (168)..(168): (n)b represent sequences complementary to the second region  of the target  RNA molecule
nguauauuaguauauuaccugguuuucgaucgaaagaucgacgaggugaaaaccucgugacaguccncaguccugauugugaucgaaagagcacagaccugaaaaggucuuucugguuucgaccagaaucagagaagngagaagucaaccagagaaacauaauauacn

SEQ ID NO: 89 - RNA, 65 nt, Artificial Sequence; identical to SEQ ID NO: 27
  Portion of SEQ ID NO. 88 between (n)a and (n)x

SEQ ID NO: 90 - RNA, 70 nt, Artificial Sequence
  Portion of SEQ ID NO. 88 between (n)x and (n)y
caguccugauugugaucgaaagagcacagaccugaaaaggucuuucugguuucgaccagaaucagagaag

SEQ ID NO: 91 - RNA, 29 nt, Artificial Sequence
  Portion of SEQ ID NO. 88 between (n)y and (n)b
gagaagucaaccagagaaacauaauauac

SEQ ID NO: 92 - RNA, 183 nt, Artificial Sequence
  S1_Tban5p_Cban3pR_Dup
gggaagucaaaccaaguauauuaguauauuaccugguuuucgaucgaaagaucgacgaggugaaaaccucgugacaguccuuagucgaaaguuuuacuagagucaguccagaaaucucccucacuucggugagggagauuucugagaagacaccagagaaacauaauauacaucgaaaaccgg

SEQ ID NO: 93 - RNA, 95 nt, Artificial Sequence
  S2_Tban5p_Cban3pR_Dup
gggaagucaaaccaaguauauuaguauauuaccuggugucccacgggcgcagaagagaagucaaccagagaaacauaauauacaucgaaaaccgg

SEQ ID NO: 94 - RNA, 182 nt, Artificial Sequence
  S1_Tlet7f_Cban3pR_Dup
gggaaacuauacaauguauauuaguauauuaccugguuuucgaucgaaagaucgacgaggugaaaaccucgugacaguccuuagucgaaaguuuuacuagagucaguccagaaaucucccucacuucggugagggagauuucugagaagacaccagagaaacauaauauaccuacuaccuca

SEQ ID NO: 95 - RNA, 94 nt, Artificial Sequence
  S2_Tlet7f_Cban3pR_Dup
gggaaacuauacaauguauauuaguauauuaccuggugucccacgggcgcagaagagaagucaaccagagaaacauaauauaccuacuaccuca

SEQ ID NO: 96 - RNA, 180 nt, Artificial Sequence
  S1_T_Orf1ab_20_Cban3pR_Dup
gggagcagccauuaguauauuaguauauuaccugguuuucgaucgaaagaucgacgaggugaaaaccucgugacaguccuuagucgaaaguuuuacuagagucaguccagaaaucucccucacuucggugagggagauuucugagaagacaccagagaaacauaauauacgaucuguccc

SEQ ID NO: 97 - RNA, 92 nt, Artificial Sequence
  S2_T_Orf1ab_20_Cban3pR_Dup
gggagcagccauuaguauauuaguauauuaccuggugucccacgggcgcagaagagaagucaaccagagaaacauaauauacgaucuguccc

SEQ ID NO: 98 - RNA, 200 nt, Artificial Sequence
  S1_T_Orf1ab_40_Cban3pR_Dup
gggaauuagguuucuuaauaguaaguauauuaguauauuaccugguuuucgaucgaaagaucgacgaggugaaaaccucgugacaguccuuagucgaaaguuuuacuagagucaguccagaaaucucccucacuucggugagggagauuucugagaagacaccagagaaacauaauauacgacuagaauugucuacuccc

SEQ ID NO: 99 - RNA, 112 nt, Artificial Sequence
  S2_T_Orf1ab_40_Cban3pR_Dup
gggaauuagguuucuuaauaguaaguauauuaguauauuaccuggugucccacgggcgcagaagagaagucaaccagagaaacauaauauacgacuagaauugucuacuccc

SEQ ID NO: 100 - RNA, 220 nt, Artificial Sequence
  S1_T_Egene_60_Cban3pR_Dup
gggauuuuacaagacucacguuaacaauauugcaguauauuaguauauuaccugguuuucgaucgaaagaucgacgaggugaaaaccucgugacaguccuuagucgaaaguuuuacuagagucaguccagaaaucucccucacuucggugagggagauuucugagaagacaccagagaaacauaauauacgcaguacgcacacaaucgaagcgcaguccc

SEQ ID NO: 101 - RNA, 132 nt, Artificial Sequence
  S2_T_Egene_60_Cban3pR_Dup
gggauuuuacaagacucacguuaacaauauugcaguauauuaguauauuaccuggugucccacgggcgcagaagagaagucaaccagagaaacauaauauacgcaguacgcacacaaucgaagcgcaguccc

SEQ ID NO: 102 - RNA, 24 nt, Artificial Sequence
  RNA T7 promoter sequence
guauaauacgacucacuauaggga